(19) United States
(12) Patent Application Publication
(10) Pub. No.: US 2026/0108602 A1
(43) Pub. Date: Apr. 23, 2026

(54) METHODS AND SYSTEMS FOR IMPROVING ANTI-CANCER THERAPIES

(71) Applicants: Osasuna Therapeutics SA, Lausanne (CH); Institut National De La Santé Et De La Recherche Médicale, Paris (FR); Université Paris Cité, Paris (FR); Sorbonne Université, Paris (FR); Assistance Publique-Hôpitaux De Paris (APHP), Paris (FR); Institut Gustave Roussy, Villejuif Cedex (FR)

(72) Inventors: Mark DE BOER, Lausanne (CH); Guido KROEMER, Paris (FR); Léa MONTEGUT, Paris (FR); Isabelle MARTINS, Paris (FR); Hui PAN, Paris (FR); Hui CHEN, Paris (FR); Sijing LI, Paris (FR)

(21) Appl. No.: 19/361,314

(22) Filed: Oct. 17, 2025

Related U.S. Application Data
(63) Continuation of application No. PCT/IB24/53769, filed on Apr. 17, 2024.

(30) Foreign Application Priority Data
Apr. 18, 2023 (EP) 23168612.2

(51) Int. Cl. A61K 39/395; A61K 31/282; A61K 31/573; A61K 39/00; A61P 35/00

(52) U.S. Cl. CPC A61K 39/3955; A61K 31/282; A61K 31/573; A61P 35/00; A61K 2039/507

(57) ABSTRACT

The present disclosure provides improved methods, compositions, agents, therapeutic regimens, and systems for cancer treatment, such as improving the outcome of chemo- and/or immunotherapies, and/or inducing autophagy in subjects through the inhibition of extracellular diazepam binding inhibitor (DBI) by various methods.

### CROSS REFERENCE TO RELATED APPLICATIONS

This application is a continuation of International Application No. PCT/IB2024/053769, filed Apr. 17, 2024, which claims priority to European Application Serial No. EP 23168612.2, filed on Apr. 18, 2023, the disclosures of which are incorporated herein by reference in their entirety.

### SEQUENCE LISTING

[001.1] The instant application contains a Sequence Listing which has been submitted electronically in XML format and is hereby incorporated by reference in its entirety. Said XML copy, created on Dec. 22, 2025, is named 206008-703301_SL.xml and is 4,363 bytes in size.

### TECHNICAL FIELD

The present disclosure relates to methods, compositions, agents, therapeutic regimens, and systems for improving anti-cancer therapies such as chemo- and/or immuno-anti-cancer therapies through the inhibition of diazepam binding inhibitor (DBI), for example, extracellular DBI.

### BACKGROUND

Diazepam binding inhibitor (DBI), also referred to as acyl-coenzyme A binding protein (ACBP), is a protein that is ubiquitously expressed in all tissues of the human body and can be released into circulation. DBI plasma concentrations increase with age and also correlate with body mass index; cardiometabolic risk factors (such as high fasting glucose levels, systolic blood pressure, total free cholesterol, triglycerides and subnormal high density lipoprotein levels); signs of liver damage (such as high circulation transaminase levels); reduced renal function (such as high creatine levels or reduced clearance); as well as signs of systemic inflammation (such as high plasma levels of interleukin-1 beta or tumor necrosis factor levels); and failure to control viral infections (such as HIV-1 and SARS-CoV2).

Neutralization (i.e., inhibition) of extracellular DBI by means of a monoclonal antibody (mAb) protects different organs (such as the heart, liver, and lungs) against damage by a variety of insults including toxic diets (e.g., high-fat diets, methionine/choline deficient diets, etc.); drugs (e.g., anthracyclines, bleomycin, paracetamol, etc.); toxins (e.g., carbon tetrachloride, concanavalin A, etc.); and ischemia or other types of physical damage (such as bile duct ligation, in the case of the liver). These organ-protective effects involve reduced cell loss or cyto-protection, reduced inflammation, and inhibition of fibrosis. Mechanistically, they have been linked to the induction of autophagy. Indeed, inhibition of autophagy by injection of high dose 3-hydroxychloroquine of knockout autophagy-related genes (such as Atg4b at the whole body level or Atg7 in specific cell types) abolishes the organ protective effects of DBI neutralization.

The pharmacological treatment of cancer involves chemotherapy, so-called targeted therapies and immunotherapies. The long-term success of all drug-based anti-cancer therapies relies on their capacity to induce anti-tumor immune responses. Thus, chemotherapies are particularly efficient if they induce immunogenic cell death (ICD) of malignant cells. For example, mitoxantrone and oxaliplatin exemplify such ICD-inducing chemotherapies that elicit potent anti-cancer immune responses.

Most immunotherapies are designed to modulate, enhance, or induce the cancer-immunity dialogue, for instance by blocking the inhibitory interaction between PD-1 and PD-L1, which constitutes an immune checkpoint. Hence, immune checkpoint inhibitors (ICI) are often agents, such as antibodies, antibody fragments or small molecules, that target, for example, PD-1 and PD-L1.

Chemo-therapies and immunotherapies are often combined as chemo-immunotherapies. In cancer patients, immunotherapies including those targeting immune checkpoints such as PD-1 and PD-L1 frequently cause unwanted inflammatory and autoimmune side effects that need to be treated by glucocorticoids (also referred to as corticosteroids or corticotherapy). However, glucocorticoids are immunosuppressive and their administration to cancer patients treated with immunotherapy (e.g., treatment with ICIs) is associated with poor outcomes as they also inhibit the anti-tumor effects of ICI treatment. Moreover, psychological or treatment-induced stress, as well as disease-related discomfort and pain, can cause an increase in the production of endogenous glucocorticoids due to the activation of the hypothalomo-pituitary-adrenal axis, thereby leading to immunosuppression and compromising the efficacy of anti-cancer treatments.

Hepatocellular carcinoma (HCC), the third leading cause of cancer-related deaths worldwide, is linked to a series of established risk factors that include metabolic dysregulation (such as obesity and diabetes favoring the development of metabolic dysfunction associated steatohepatitis (MASH), and progression to advanced fibrosis and cirrhosis), repeated insult by hepatotoxins (such as alcohol and aflatoxin) as well as infection by hepatotropic viruses (such as hepatitis viruses B and C). In spite of the ever-more-detailed comprehension of the molecular and cellular pathways leading to HCC development, there are rather few successful treatment options. Curative attempts (e.g., transplantation, resection and thermal ablation), are usually limited to patients with early-stage HCC (BCLC-0 and BCLC-A) while treatments like transarterial chemoembolization, transarterial radioembolization and systemic treatments (tyrosine-kinase inhibitors and immunotherapy), are usually reserved for intermediate (BCLC-B) and advance stage (BCLC-C) patients.

Thus, there remains a need to provide new and improved anti-cancer chemo-, immuno- and/or chemo-immunotherapies, as well as methods of treating HCC, that overcome at least some of the above issues, at least to some extent.

### BRIEF SUMMARY

Applicants surprisingly found that administering an agent that stimulates autophagy by inhibiting extracellular human diazepam binding inhibitor (DBI) (anti-DBI agent) can improve a therapeutic effect of immunotherapy, chemotherapy and chemo-immunotherapy.

Applicants have also surprisingly found that in various tumor models, neutralization or inhibition of extracellular diazepam binding inhibitor (DBI), also referred to as the acyl coenzyme A binding protein (ACBP), by various methods improves the outcome of immunotherapy, chemotherapy and chemo-immunotherapy, resulting in an improved reduction in tumor growth, a higher cure rate, and/or extension of animal survival.

Neutralization/inhibition of extracellular DBI can be achieved in a number of ways, for example by vaccination to induce neutralizing autoantibodies or by administration of an agent such as a monoclonal antibody (mAb) that binds extracellular DBI or modulates the activity of extracellular DBI.

Applicants have additionally surprisingly found that extracellular DBI inhibition enhanced cancer immunosurveillance. This was demonstrated by a change in the composition of the tumor immune infiltrate with an augmented ratio of cytotoxic T cells over regulatory T cells, effects on T cell activation and exhaustion markers.

Applicants further surprisingly found that extracellular DBI inhibition reverses the immunosuppressive effects of corticotherapy in that, to the extent any therapeutic efficacy of immunotherapy, chemotherapy or chemo-immunotherapy that was reduced or diminished potentially as an outcome of co-administration of corticotherapy agents, a complete or partial restoration of such efficacy was observed when the therapy further included inhibition of extracellular DBI. Applicants' data show that in the presence of an anti-DBI agent the inhibition of immune checkpoint inhibitor (ICI) treatment by corticosteroids is overcome, i.e., the anti-DBI agent protects T cells, that are activated by the ICI, from being inhibited. Mouse experiments have shown that injection of corticosteroids increases the level of extracellular DBI. Without wishing to be bound by theory, Applicant submits that extracellular DBI has a direct or indirect inhibitory effect on T cells.

Mammalian subjects, for instance mice being treated with corticotherapy agents and an anti-DBI agent showed better vaccination responses against chemotherapy-killed cancer, showing that the capacity of dead-cell vaccines to control the progression of tumors was enhanced.

Disclosed herein are methods of improving a therapeutic effect of immunotherapy in a subject, the methods comprising administering to the subject: (a) an agent that stimulates autophagy by inhibiting extracellular human diazepam binding inhibitor (DBI) (anti-DBI agent); and (b) one or more immunotherapy agents, wherein administration of the anti-DBI agent and the one or more immunotherapy agents is sufficient to improve the therapeutic effects of immunotherapy relative a comparable method in the absence of the administration of the anti-DBI agent. Agents, compositions and combinations for use in such methods are also disclosed. Thus, according to one aspect of the invention, there is provided an agent that stimulates autophagy by inhibiting extracellular human diazepam binding inhibitor (DBI), or a composition comprising such an agent, for use in such methods. According to a further aspect of the invention, there is provided one or more immunotherapy agents (or a composition or compositions comprising such agents) for use in such methods. According to a still further aspect of the invention, there is provided a combination of an agent that stimulates autophagy by inhibiting extracellular human diazepam binding inhibitor (DBI) and one or more immunotherapy agents for use in such methods.

Disclosed herein are methods of treating cancer in a subject in need thereof, the methods comprising administering to the subject: (a) an agent directed against extracellular human diazepam binding inhibitor (DBI) (anti-DBI agent) in an amount sufficient to inhibit extracellular human DBI; and (b) one or more anti-cancer agents; wherein administration of the anti-DBI agent and the one or more anti-cancer agents is in a therapeutic amount sufficient to treat cancer and results in enhanced treatment of the cancer in the subject as compared to a comparable method in the absence of the administration of the anti-DBI agent. Agents, compositions and combinations for use in such methods are also disclosed. Thus, according to one aspect of the invention, there is provided an agent directed against extracellular human diazepam binding inhibitor (DBI), or a composition comprising such an agent, for use in such methods. According to a further aspect of the invention, there is provided one or more immunotherapy agents (or a composition or compositions comprising such agents) for use in such methods. According to a still further aspect of the invention, there is provided a combination of an agent directed against extracellular human diazepam binding inhibitor (DBI) and one or more immunotherapy agents for use in such methods.

In some embodiments, the methods disclosed herein further comprise administering one or more corticotherapy agents. In some embodiments, the methods disclosed herein reduce the immunosuppressive effects of the one or more corticotherapy agents in the subject undergoing immunotherapy or treatment of cancer relative to a comparable method in the absence of the administration of the anti-DBI agent.

Disclosed herein are methods of enhancing immunosurveillance in a subject having cancer to enhance the therapeutic effect of an anti-cancer therapy, the methods comprising administering to the subject: (a) an agent directed against extracellular human diazepam binding inhibitor (DBI) (anti-DBI agent) in an amount sufficient to inhibit extracellular human DBI; and (b) one or more anti-cancer agents, wherein administration of the anti-DBI agent and the one or more anti-cancer agents is sufficient to induce one or more immunosurveillance biomarkers resulting in an enhanced therapeutic effect of the anti-cancer therapy relative a comparable method in the absence of the administration of the anti-DBI agent. Agents, compositions and combinations for use in such methods are also disclosed. Thus, according to one aspect of the invention, there is provided an agent directed against extracellular human diazepam binding inhibitor (DBI), or a composition comprising such an agent, for use in such methods. According to a further aspect of the invention, there is provided one or more immunotherapy agents (or a composition or compositions comprising such agents) for use in such methods. According to a still further aspect of the invention, there is provided a combination of an agent directed against extracellular human diazepam binding inhibitor (DBI) and one or more immunotherapy agents for use in such methods.

Disclosed herein are methods of reducing the immunosuppressive effects of corticotherapy in a subject undergoing immunotherapy, the methods comprising administering to the subject: (a) an agent directed against extracellular human diazepam binding inhibitor (DBI) (anti-DBI agent) in an amount sufficient to inhibit extracellular human DBI; and (b) one or more corticotherapy agents; wherein administration of the anti-DBI agent and the one or more corticotherapy agents is sufficient to reduce the immunosuppressive effects of the corticotherapy in the subject undergoing immunotherapy relative to a comparable method in the absence of the administration of the anti-DBI agent. In some embodiments, the methods disclosed herein further comprise administering one or more anti-cancer agents. Agents, compositions and combinations for use in such methods are also disclosed. Thus, according to one aspect of the invention, there is provided an agent directed against extracellular human diazepam binding inhibitor (DBI), or a composition comprising such an agent, for use in such methods. According to a further aspect of the invention, there is provided one or more corticotherapy agents (or a composition or compositions comprising such agents) for use in such methods. According to a still further aspect of the invention, there is provided a combination of an agent directed against extracellular human diazepam binding inhibitor (DBI) and one or more corticotherapy agents for use in such methods.

In some embodiments of the methods disclosed herein, the anti-DBI agent is an antibody or an aptamer.

In some embodiments of the methods disclosed herein, the one or more anti-cancer agents comprise a chemotherapy agent, an immunotherapy agent, or both. In some embodiments, the chemotherapy agent comprises an immunogenic cell death (ICD) inducing activity. In some embodiments, the chemotherapy agent comprises alkylating agents, alkyl sulfonates, aziridines, ethylenimines, methylamelamines, altretamine, triethylenemelamine, trietylenephosphoramide, triethiylenethiophosphoramide, trimethylolomelamine, acetogenins, a camptothecin, bryostatin; callystatin; CC-1065, cryptophycins, dolastatin, duocarmycin, eleutherobin, pancratistatin, a sarcodictyin, spongistatin, nitrogen mustards, nitrosureas, antibiotics, dynemicin, bisphosphonates, esperamicin, neocarzinostatin chromophore, related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine; doxorubicin, epirubicin, esorubicin, idarubicin, marcellomycin, mitomycins, anti-metabolites, folic acid analogues, purine analogues, pyrimidine analogues, androgens, anti-adrenals, folic acid replenisher, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone, 2,2′,2″-trichlorotriethylamine, trichothecenes, urethan, vindesine, dacarbazine, mannomustine, mitobronitol, mitolactol, pipobroman, gacytosine, arabinoside, cyclophosphamide, thiotepa, toxoids, chlorambucil, gemcitabine, 6-thioguanine, mercaptopurine, methotrexate, platinum coordination complexes, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylomithine (DMFO), retinoids, capecitabine, pharmaceutically acceptable salts, acids or derivatives of any of the foregoing, or any combinations thereof. In some embodiments, the chemotherapy agent comprises thiotepa, cyclosphosphamide, busulfan, improsulfan, piposulfan, benzodopa, carboquone, meturedopa, bullatacin, bullatacinone, topotecan, adozelesin, carzelesin, bizelesin synthetic analogues, cryptophycin 1, cryptophycin 8, KW-2189, CB1-TM1, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, enediyne antibiotics, calicheamicin, calicheamicin gammall, calicheamicin omegall, dynemicin A, clodronate, morpholino-doxorubicin, cyanomorpholino-doxorubicin, 2-pyrrolino-doxorubicin, deoxy doxorubicin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, maytansine, ansamitocins, T-2 toxin, verracurin A, roridin A, anguidine, paclitaxel, doxetaxel, cisplatin, oxaliplatin, carboplatin, CPT-1 1, retinoic acid, sorafenib, olaparib, cetuximab, gefitinib, sulfasalazine, beta-elemene, trigonelline, vorinostat, sunitinib, artesunate, pharmaceutically acceptable salts, acids or derivatives of any of the foregoing, or any combinations thereof.

In some embodiments, the one or more anti-cancer agents can be a ferroptosis-inducing agent. For example, an agent that reduces the activity or expression of DBI as described herein (for example, agents that reduce the activity of DBI described herein such as anti-DBI antibodies, or agents that reduced expression of DBI described herein such as siRNA or thyroid hormone receptor agonists (e.g., resmetirom)) can be used in combination with a ferroptosis-inducing agent such as sorafenib, olaparib, cetuximab, gefitinib, sulfasalazine, beta-elemene, trigonelline, vorinostat, sunitinib, or artesunate. In some instances, the agent that reduces the activity or expression of DBI improves the activity of the ferroptosis-inducing agent against a target cancer cell, relative to the activity of the ferroptosis-inducing agent in the absence of the agent the reduces the activity or expression of DBI. In some instances, the the agent that reduces the activity or expression of DBI displays a synergistic improvement in the activity of the ferroptosis-inducing agent against a target cancer cell, relative to the activity of the ferroptosis-inducing agent in the absence of the agent the reduces the activity or expression of DBI.

In some embodiments of the methods disclosed herein, the one or more immunotherapy agents comprise activity against an immune checkpoint. In some embodiments, the immune checkpoint comprises PD1 (programmed death 1), PDL1 (programmed cell death-ligand 1), CTLA4 (cytotoxic T-lymphocyte-associated protein 4), PDL2 (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG, CBLB (Casitas b-lineage lymphoma Proto-Oncogene B), CISH (Cytokine Inducible SH2 Containing Protein), or any combinations thereof.

In some embodiments of the methods disclosed herein, the one or more immunotherapy agents comprise an anti-PD1 agent, anti-PD-L1 agent, anti-CTLA4 agent, anti-PD-L2 agent, anti-KIR agent, anti-B7-H3 agent, anti-B7-H4 agent, anti-BTLA agent, anti-LAG3 agent, anti-TIM-3 agent, anti-VISTA agent, anti-ILT2/LILRB1 agent, anti-ILT3/LILRB4 agent, anti-ILT4/LILRB2 agent, anti-TIGIT agent, anti-NKG2A agent, anti-PVRIG agent, anti-CBLB agent, anti-CISH agent, or any combinations thereof. In some embodiments, the immunotherapy agent comprises an anti-PD1 antibody, anti-PD-L1 antibody, anti-CTLA4 antibody, anti-PD-L2 antibody, anti-KIR antibody, anti-B7-H3 antibody, anti-B7-H4 antibody, anti-BTLA antibody, anti-LAG3 antibody, anti-TIM-3 antibody, anti-VISTA antibody, anti-ILT2/LILRB1 antibody, anti-ILT3/LILRB4 antibody, anti-ILT4/LILRB2 antibody, anti-TIGIT antibody, anti-NKG2A antibody, anti-PVRIG antibody, anti-CBLB antibody, anti-CISH antibody, ipilimumab, tremelimumab, MK-1308, FPT155, PRS010, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002, CP675206, pembrolizumab, nivolumab, pidilizumab, AMP-224, BMS-936559, cemiplimab, PDR001, MDX-1105, MEDI4736, atezolizumab, avelumab, BMS-936559, durvalumab, lirlumab (IPH2102), IPH2101, MGA271, FPA150, IMP321 (eftilagimod alpha), relatlimab, MK-4280, AVA017, BI754111, ENUM006, GSK2831781, INCAGN2385, LAG3Ig, LAG525, REGN3767, Sym016, Sym022, Sym023, TSR033, TSR075, XmAb22841. LY3321367, MBG453, TSR-022, JNJ-61610588, MK-7684, PTZ-201, RG6058, COM902, IPH-2201, COM701, CA-327, LAG525, REGN3767, BI 754111, tebotelimab, FS118, MGC018, or any combinations thereof. In some embodiments, the immunotherapy agent comprises an anti-PD1 antibody, anti-PD-L1 antibody, anti-CTLA4 antibody, or any combinations thereof.

In some embodiments of the methods disclosed herein, the one or more corticotherapy agents are selected from cortisol, cortisone, prednisone, prednisolone, methylprednisolone, dexamethasone, betamethasone, triamcinolone, deflazacort, fludrocortisone acetate, deoxycorticosterone acetate, aldosterone, beclomethasone, and any combinations thereof.

In some embodiments of the methods disclosed herein, the cancer comprises an inhibitory tumor microenvironment.

In some embodiments, the methods disclosed herein further comprise inducing at least one immunosurveillance biomarker comprising: increased CD8⁺ expression, decreased CD4⁺ expression, decreased T_{reg} cells (CD4⁺Foxp3⁺), an increased ratio of CD8⁺ T cells to T_{reg} cells (CD4⁺Foxp3⁺), increased T_{H}+ cells (CD4⁺Foxp3⁻), decreased expression of Lag3⁺ in T_{H}+ cells (CD4⁺Foxp3⁻), decreased expression of Lag3⁺ in CD8⁺ cells, decreased expression of Foxp3⁺ in CD4⁺ cells, decreased T_{reg} ICOS⁺ GITR⁺ Lag3⁻ CD4⁺ cells, or combinations thereof, in a sample relative to a comparable method in the absence of the administration of the anti-DBI agent. In some embodiments, the inducing of the at least one immunosurveillance biomarker comprises increased CD8⁺ expression relative to a comparable method in the absence of the administration of the anti-DBI agent. In some embodiments, the inducing of the at least one immunosurveillance biomarker comprises decreased CD4⁺ expression a comparable in the absence of the administration of the anti-DBI agent. In some embodiments, the inducing of the at least one immunosurveillance biomarker comprises decreased T_{reg} cells (CD4⁺Foxp3⁺) relative to a comparable method in the absence of the administration of the anti-DBI agent. In some embodiments, the inducing of the at least one immunosurveillance biomarker comprises an increased ratio of CD8⁺ T cells to T_{reg} cells (CD4⁺Foxp3⁺) relative to a comparable method in the absence of the administration of the anti-DBI agent. In some embodiments, the inducing of the at least one immunosurveillance biomarker comprises increased incidence of T_{H}+ cells (CD4⁺Foxp3⁻) relative to a comparable method in the absence of the administration of the anti-DBI agent. In some embodiments, the inducing of the at least one immunosurveillance biomarker comprises decreased expression of Lag3⁺ in T_{H}+ cells (CD4⁺Foxp3⁻) relative to a comparable method in the absence of the administration of the anti-DBI agent. In some embodiments, the inducing of the at least one immunosurveillance biomarker comprises decreased expression of Lag3⁺ in CD8⁺ cells relative to a comparable method in the absence of the administration of the anti-DBI agent. In some embodiments, the inducing of the at least one immunosurveillance biomarker comprises decreased expression of Foxp3⁺ in CD4⁺ cells relative to a comparable method in the absence of the administration of the anti-DBI agent. In some embodiments, the inducing of the at least one immunosurveillance biomarker comprises decreased T_{reg} ICOS⁺ GITR⁺ Lag3⁻ CD4⁺ cells relative to a comparable method in the absence of the administration of the anti-DBI agent.

In some embodiments, the methods disclosed herein maintain the ratio of CD8⁺ T cells to T_{H}+ cells (CD4⁺Foxp3⁻) cells relative to a comparable method in the absence of the administration of the anti-DBI agent. In some embodiments, the methods disclosed herein maintain the ratio of CD8⁺ T cells to CD4⁺ T cells relative to a comparable method in the absence of the administration of the anti-DBI agent. In some embodiments, the methods disclosed herein maintain the incidence of T_{H} ICOS^{int} GITR⁺ Lag3⁻ PD1⁻ CD4⁺ T cells relative to a comparable method in the absence of the administration of the anti-DBI agent.

In some embodiments, the methods disclosed herein further comprise inhibiting of at least one cancer progression biomarker comprising: a decreased cancer amount, decreased proliferation of cancer cells, increased cancer cell death, decreased cancer growth, or combinations thereof, of the subject relative to a comparable method in the absence of the administration of the anti-DBI agent. In some embodiments, the inhibiting of the at least one cancer progression biomarker comprises a decreased cancer amount and wherein the decreased cancer amount comprises a decrease in cancer area or cancer volume. In some embodiments, the decreased cancer amount comprises decreased cancer volume relative to a comparable method in the absence of the administration of the anti-DBI agent at a comparable time point. In some embodiments, the cancer volume is decreased up to about 60%. In some embodiments, the decreased cancer amount comprises decreases cancer area relative to a comparable method in the absence of the administration of the anti-DBI agent at a comparable time point. In some embodiments, the inhibiting of the at least one cancer progression biomarker comprises decreased proliferation of cancer cells relative to a comparable method in the absence of the administration of the anti-DBI agent at a comparable time point. In some embodiments, the inhibiting of the at least one cancer progression biomarker comprises increased cancer cell death relative to a comparable method in the absence of the administration of the anti-DBI agent at a comparable time point. In some embodiments, the inhibiting of the at least one cancer progression biomarker comprises decreased cancer growth relative to a comparable method in the absence of the administration of the anti-DBI agent at a comparable time point.

In some embodiments, the methods disclosed herein further comprise increased incidence of survival of the subject relative to a comparable method in the absence of the administration of the anti-DBI agent at a comparable time point. In some embodiments, the incident of survival is increased up to about 50% relative to a comparable method in the absence of the administration of the anti-DBI agent at a comparable time point.

In some embodiments, the methods disclosed herein further comprise incidence of cancer free occurrence in the subject relative to a comparable method in the absence of the administration of the anti-DBI agent at a comparable time point. In some embodiments, the incidence of cancer free occurrence is increased up to 40% relative to a comparable method in the absence of the administration of the anti-DBI agent at a comparable time point.

In some embodiments, the methods disclosed herein further comprise increased time to fatality of the subject relative to a comparable method in the absence of the administration of the anti-DBI agent at a comparable time point.

In some embodiments of the methods disclosed herein, the cancer is a refractory cancer. In some embodiments, the cancer is resistant to immune checkpoint inhibitor therapy. In some embodiments, the cancer is selected from solid tumors, blood-borne tumors, cancers of skin, cancers of tissues, cancers of organs, cancers of bone, cancers of cartilage, cancers of blood, cancers of vessels, primary cancers, metastatic cancers, bladder cancer, bone marrow cancer, brain cancer, breast cancer, colon cancer, esophagus cancer, gastro-intestine cancer, gum cancer, kidney cancer, liver cancer, lung cancer, nasopharynx cancer, neck cancer, ovary cancer, prostate cancer, stomach cancer, testis cancer, tongue cancer, and uterus cancer. In some embodiments, the cancer is selected from carcinomas, lung cancer, non-small cell lung cancer, breast cancer, neoplasm carcinoma, undifferentiated carcinoma, giant cell carcinoma, spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary adenocarcinoma, follicular adenocarcinoma, non-encapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, Paget's disease, mammary carcinoma, acinar cell carcinoma, adenosquamous carcinoma, adenocarcinoma with squamous metaplasia, thymoma, ovarian stromal tumor, thecoma, granulosa cell tumor, roblastoma, Sertoli cell carcinoma, leydig cell tumor, lipid cell tumor, paraganglioma, extra-mammary paraganglioma, pheochromocytoma, glomangiosarcoma, melanoma, amelanotic melanoma, superficial spreading melanoma, melanoma in giant pigmented nevus, epithelioid cell melanoma, blue nevus, sarcoma, fibrosarcoma, fibrous histiocytoma, myxosarcoma, liposarcoma, leiomyosarcoma, rhabdomyosarcoma, embryonal rhabdomyosarcoma, alveolar rhabdomyosarcoma, stromal sarcoma, mixed tumor, mullerian mixed tumor, nephroblastoma, hepatoblastoma, carcinosarcoma, mesenchymoma, brenner tumor, phyllodes tumor, synovial sarcoma, mesothelioma, dysgerminoma, embryonal carcinoma, teratoma, struma ovarii, choriocarcinoma, mesonephroma, hemangiosarcoma; hemangioendothelioma, kaposi's sarcoma, hemangiopericytoma, lymphangiosarcoma, osteosarcoma, juxtacortical osteosarcoma, chondrosarcoma, chondroblastoma, mesenchymal chondrosarcoma, giant cell tumor of bone, ewing's sarcoma, odontogenic tumor, ameloblastic odontosarcoma, ameloblastoma, ameloblastic fibrosarcoma, pinealoma, chordoma, glioma, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, neurofibrosarcoma, neurilemmoma, granular cell tumor, lymphoma, Hodgkin's disease, Hodgkin's lymphoma, paragranuloma, lymphoma, small lymphocytic, large cell diffuse lymphoma, follicular lymphoma, mycosis fungoides, other specified non-Hodgkin's lymphomas, histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia. In some embodiments, the cancer is a malignant cancer. In some embodiments, the cancer is selected from carcinomas, lung cancer, non-small cell lung cancer, and breast cancer. In some embodiments, the cancer is a solid tumor cancer. In some embodiments, the cancer is a fibrous tumor.

In some embodiments of the methods disclosed herein, the subject is human.

Disclosed herein are therapeutic regimens for treating cancer comprising: (a) an agent directed against extracellular human diazepam binding inhibitor (DBI) (anti-DBI agent) in an amount sufficient to inhibit extracellular human DBI; and (b) one or more anti-cancer agents, wherein the anti-DBI agent and the one or more anti-cancer agents are present in the therapeutic regimen in an amount sufficient to inhibit one or more cancer progression biomarkers in a subject upon administration to the subject relative to a comparable regimen absent the administration of the anti-DBI agent. Agents, compositions and combinations for use in such regimens are also disclosed. Thus, according to one aspect of the invention, there is provided an agent directed against extracellular human diazepam binding inhibitor (DBI), or a composition comprising such an agent, for use in such regimens. According to a further aspect of the invention, there is provided one or more anti-cancer agents (or a composition or compositions comprising such agents) for use in such regimens. According to a still further aspect of the invention, there is provided a combination of an agent directed against extracellular human diazepam binding inhibitor (DBI) and one or more anti-cancer agents for use in such regimens.

In some embodiments of the therapeutic regimens disclosed herein, the anti-DBI agent is an antibody or an aptamer.

In some embodiments of the therapeutic regimens disclosed herein, the one or more anti-cancer agents comprise a chemotherapy agent, an immunotherapy agent, or a combination thereof. In some embodiments, the chemotherapy agent comprises an immunogenic cell death (ICD) inducing activity. In some embodiments, the immunotherapy agent comprises activity against an immune checkpoint. In some embodiments, the immune checkpoint comprises PD1 (programmed death 1), PDL1 (programmed cell death-ligand 1), CTLA4 (cytotoxic T-lymphocyte-associated protein 4), PDL2 (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG, CBLB, CISH, or any combinations thereof.

In some embodiments of the therapeutic regimens disclosed herein, the immunotherapy agent comprises an anti-PD1 agent, anti-PD-L1 agent, anti-CTLA4 agent, anti-PD-L2 agent, anti-KIR agent, anti-B7-H3 agent, anti-B7-H4 agent, anti-BTLA agent, anti-LAG3 agent, anti-TIM-3 agent, anti-VISTA agent, anti-ILT2/LILRB1 agent, anti-ILT3/LILRB4 agent, anti-ILT4/LILRB2 agent, anti-TIGIT agent, anti-NKG2A agent, anti-PVRIG agent, anti-CBLB agent, anti-CISH agent, or any combinations thereof. In some embodiments, the immunotherapy agent comprises ipilimumab, tremelimumab, MK-1308, FPT155, PRS010, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002, CP675206, pembrolizumab, nivolumab, pidilizumab, AMP-224, BMS-936559, cemiplimab, PDR001, MDX-1105, MEDI4736, atezolizumab, avelumab, BMS-936559, durvalumab, lirlumab (IPH2102), IPH2101, MGA271, FPA150, IMP321 (eftilagimod alpha), relatlimab, MK-4280), AVA017, BI754111, ENUM006, GSK2831781, INCAGN2385, LAG3Ig, LAG525, REGN3767, Sym016, Sym022, Sym023, TSR033, TSR075, XmAb22841, LY3321367, MBG453, TSR-022, JNJ-61610588; MK-7684, PTZ-201, RG6058, COM902, IPH-2201, COM701, CA-327, LAG525, REGN3767, BI 754111, tebotelimab, FS118, MGC018, or any combinations thereof.

In some embodiments, the therapeutic regimens disclosed herein further comprise one or more corticotherapy agents. In some embodiments, the one or more corticotherapy agents are selected from cortisol, cortisone, prednisone, prednisolone, methylprednisolone, dexamethasone, betamethasone, triamcinolone, deflazacort, fludrocortisone acetate, deoxycorticosterone acetate, aldosterone, beclometasone, and any combinations thereof.

In some embodiments, of the therapeutic regimens disclosed herein, the anti-DBI agent and the one or more anti-cancer agents are separate components that are administered together or sequentially. In some embodiments, the anti-DBI agent is administered at least about two weeks prior to the one or more anti-cancer agents. In some embodiments, the anti-DBI agent and the one or more anti-cancer agents are co-administered. In some embodiments, the one or more corticotherapy agents are co-administered with the anti-DBI agent and/or the one or more anti-cancer agents: or are administered sequentially. In some embodiments, any two or more of the anti-DBI agent, the one or more anti-cancer agents, and the one or more corticotherapy agents are co-administered.

Disclosed herein are systems comprising one or more components, wherein the one or more components individually comprise one or more of the following: (a) an agent directed against extracellular human diazepam binding inhibitor (DBI) (anti-DBI agent) in an amount sufficient to inhibit extracellular DBI in a subject; (b) one or more anti-cancer agents, optionally wherein the one or more anti-cancer agents comprises a chemotherapy agent, an immunotherapy agent, or a combination thereof; (c) one or more corticotherapy agents; (d) or any combinations of (a)-(c).

### FIGURE DESCRIPTIONS

FIG. 1A-FIG. 1D: Improvement of chemo-immunotherapeutic treatment of MCA205 carcinomas by autoimmunization against ACBP/DBI. Anti-ACBP/DBI autoimmunity was induced in C57BL/6J mice by four weekly doses of KLH-ACBP/DBI vaccine or its non ACBP/DBI-specific control KLH. After two weeks of immune recovery, mice were injected subcutaneously with MCA205 cells and followed the chemo-immunotherapeutic cycle described in FIG. 1A, consisting of one dose of oxaliplatin (OXA; 10 mg/kg) and three consecutive doses of anti-PD1 monoclonal antibody (αPD1; 200 μg per dose), or were injected with the matching vehicle and isotype mAb. Average (mean±SEM) tumor growth curves are reported in FIG. 1B; and overall survival is reported in FIG. 1C. Statistical analyses were performed by linear mixed effect modeling of tumor sizes for longitudinal analysis and by two-by-two log-rank Mantel-Cox test for survival (GraphPad Prism). The computed p-values are presented in the table shown in FIG. 1D and formatted in bold font when under the threshold of 0.05. TF: Tumor Free.

FIG. 2A-FIG. 2D: Improvement of chemo-immunotherapeutic treatment of MCA205 carcinomas by injection of ACBP/DBI-neutralizing monoclonal antibody. C57Bl/6J mice were injected subcutaneously with MCA205 cells and followed the chemo-immunotherapeutic cycle described in FIG. 2A, consisting of one dose of oxaliplatin (OXA; 10 mg/kg) and three consecutive doses of anti-PD1 monoclonal antibody (αPD1, 200 μg per dose), or were injected with the matching vehicle and isotype mAb. Right before and for one week after chemotherapy, half the mice received repeated doses of anti-ACBP/DBI mAb (αDBI; 2.5 mg/kg). The reduction of tumor size obtained with chemo-immunotherapy was further improved when combining it with ACBP/DBI mAb neutralization, as shown in FIG. 2B, and overall survival was prolonged accordingly, as shown in FIG. 2C. Tumor growth and survival curves presented in FIGS. 2B and 2C are from one representative experiment, and the experiment was repeated twice yielding similar results. Statistical analyses were performed on pooled data from the two independent experiments by linear mixed effect modeling of tumor sizes for longitudinal analysis and by two-by-two log-rank Mantel-Cox test for survival (GraphPad Prism). The computed p-values are presented in the table shown in FIG. 2D and formatted in bold font when below the threshold of 0.05. TF: Tumor Free.

FIG. 3A-FIG. 3M: Immune control of MCA205 carcinomas during chemo-immunotherapeutic treatment improvement by injection of ACBP/DBI-neutralizing monoclonal antibody. T cell populations of the tumor microenvironment 10 days after chemotherapy, as shown in FIG. 3A, were analyzed by flow cytometry. The number of specific cell populations relative to the total analyzed single cells is presented for the total cytotoxic CD8⁺ T cells, as shown in FIG. 3B, and CD4⁺ T cells, as shown in FIG. 3C, T lymphocytes, and more specifically for the CD4⁺FoxP3⁺ regulatory T cells (T_{reg}), as shown in FIG. 3E, as well as the CD4⁺FoxP3⁻ helper T cells (T_{H}), as shown in FIG. 3G. Increased ratios between the counts of CD8⁺ and CD4⁺ T cells, as shown in FIG. 3D, CD8⁺ T cells and T_{reg}, as shown in FIG. 3F, and CD8⁺ T cells and T_{H}, as shown in FIG. 3H, correlate with increased cancer-directed cytotoxicity, while a high proportion of FoxP3⁺ cells within the CD4⁺ T cells, as shown in FIG. 3I, is indicative of dampened immune response against cancer. The proportion of T_{H}, as shown in FIG. 3J, and CD8⁺ T cells, as shown in FIG. 3K, cells expressing the surface marker LAG3 is a sign of the exhaustion of the helper and cytotoxic cells infiltrating the tumor. More precisely, unsupervised clustering highlights two clusters of interest in the CD4⁺ T cells polarization when combining anti-DBI to chemo-immunotherapy: a cluster of activated T_{H} (FoxP3⁻, ICOS^{int}, GITR⁺, Lag3⁻) that becomes prevalent, as shown in FIG. 3L, and a cluster of activated T_{reg} (FoxP3⁺, GITR⁺, Lag3⁻) that is suppressed, as shown in FIG. 3M, among the CD4⁺ T cells. Compensation, scaling and gating strategies were performed using the omiq.ai platform. Data from three independent experiments are presented, population relative counts were cleaned up by ROUT test (outlier threshold=1%) and p-values were calculated by one-way ANOVA with Sidak correction for multiple comparisons.

FIG. 4A-FIG. 4B: Effect of a treatment combining a monoclonal antibody neutralizing ACBP/DBI and chemotherapy in the case of TC1 lung cancer. Living TC1 cells stably expressing luciferase activity (TC1 Luc, 5×10⁵ per mouse) were intravenously (i.v.) injected into wild type C57BL/6J mice (minimum of 7 mice per group). Then mice received treatments as indicated: vehicle+isotype control antibody (CTR): monoclonal antibody to DBI (αDBI; 2.5 mg/kg): monoclonal antibody to PD1 (αPD1; 10 mg/kg) and/or oxaliplatin (OXA; 5 mg/kg). Tumor size was monitored by luciferase activity every 4 days (shown in FIG. 4A). Representative time lapse images of 3 CTR mouse, 3 αDBI−, 3 OXA+αPD1−, and 3 OXA+αDBI+αPD1− treated mice are shown in FIG. 4B. Crosses indicate that the animal had died.

FIG. 5A-FIG. 5C: Improvement of chemo-immunotherapeutic treatment of TC1 lung cancer by injection of ACBP/DBI-neutralizing monoclonal antibody. The tumor sizes of orthotopic TC1 lung cancers were quantified as the total flux of acquired bioluminescence photons. Average (mean±SEM) tumor growth curves are shown in FIG. 5A; and overall survival is shown in FIG. 5B. Statistical significance was calculated by means of the ANOVA Type 2 (Wald test) for tumor growth curves, or by two-by-two log-rank Mantel-Cox test for survival (GraphPad Prism). The computed p-values are presented in the table shown in FIG. 5C and formatted in bold font when below the threshold of 0.05. TF: Tumor Free.

FIG. 6A-FIG. 6B: Effect of a treatment combining a monoclonal antibody neutralizing ACBP/DBI and chemotherapy in a context of corticotherapy in TC1 lung cancer. Living TC1 cells stably expressing luciferase activity (TC1 Luc, 5×10⁵ per mouse) were intravenously (i.v.) injected into wild type C57BL/6J mice. Then mice received treatments as indicated: vehicle (CTR); anti-DBI antibody (αDBI; 5 mg/kg) or its isotype: anti-PD1 (αPD1; 10 mg/kg) antibody or its isotype and oxaliplatin (OXA; 5 mg/kg). Tumor size was monitored by luciferase activity every 4 days, shown in FIG. 6A. Representative time lapse images of 3 CTR mouse, 3 αPD1+OXA−, 3 OXA+αPD1+αDBI−, 3 CORT+OXA+αPD1− and CORT+OXA+αDBI+αPD1-treated mice are shown in FIG. 6B. Crosses indicate that the animal had died.

FIG. 7A-FIG. 7C: Improvement of chemo-immunotherapeutic treatment of TC1 lung cancer by injection of ACBP/DBI-neutralizing monoclonal antibody in the context of corticotherapy. The tumor sizes of orthotopic TC1 lung cancers were quantified as the total flux of acquired bioluminescence photons. Average (mean±SEM) tumor growth curves are shown in FIG. 7A; and overall survival is shown in FIG. 7B. Statistical significance was calculated by means of the ANOVA Type 2 (Wald test) for tumor growth curves, or by two-by-two log-rank Mantel-Cox test for survival (GraphPad Prism). The computed p-values are presented in the table shown in FIG. 7C and formatted in bold font when below the threshold of 0.05. TF: Tumor Free.

FIG. 8A-FIG. 8D: Improvement of immunotherapy treatment of E0771 tumor (breast cancer) by injection of ACBP/DBI-neutralizing monoclonal antibody in the absence and presence of corticotherapy. Living E0771 cells (5×10⁵ per mouse) were subcutaneously injected into the right lower quadrant of the abdomen alongside of the mammary gland (close to the orthotopic site) in C57BL/6J mice. When tumors became palpable (day 8), mice were treated with a neutralizing anti-DBI monoclonal antibody (5 mg/kg) or its isotype every two days. At the same time, free corticosterone (Sigma-Aldrich) was added to the drinking water (0.1 mg/ml; water/ethanol 0.66%). Anti-PD1 monoclonal antibody (αPD1; 10 mg/kg) was injected at days 12, 15, 18 and 21) as indicated in FIG. 8A. Average (mean±SEM) tumor growth curves are shown in FIG. 8B; and overall survival is shown in FIG. 8C. Statistical analyses were performed by linear mixed effect modeling of tumor sizes for longitudinal analysis and by two-by-two log-rank Mantel-Cox test for survival (GraphPad Prism). The computed p-values are presented in the table shown in FIG. 8D and formatted in bold font when under the threshold of 0.05. TF: Tumor Free.

FIG. 9A-FIG. 9D: Improvement of the anti-cancer immune response by injection of ACBP/DBI-neutralizing monoclonal antibody in the context of corticotherapy. C57BL/6J mice (8-weeks-old female) were pretreated with a neutralizing anti-DBI monoclonal antibody (αDBI; 5 mg/kg) or its isotype (IgG2A; 5 mg/kg) every two days (i.p., in 200 μl PBS) and free corticosterone (CORT) in the drinking water (0.1 mg/ml; ethanol/water 0.66%) two weeks before the vaccination. Wild-type MCA205 cells were treated with mitoxantrone (MTX; 4 μM) for 24 h. 2×10⁶ cells of the cellular suspension were s.c. injected to the left flank of immunocompetent C57BL/6J mice. PBS was injected as a negative control. One week later, living MCA205 cancer cells (2×10⁵ cells per mouse) were injected in the right flank of vaccinated mice. Corticosterone (CORT) and anti-ACBP/DBI were continually administrated throughout the experiment shown in FIG. 9A. Average (mean±SEM) tumor growth curves are shown in FIG. 9B; and overall survival is shown in FIG. 9C. Statistical analyses were performed by linear mixed effect modeling of tumor sizes for longitudinal analysis and by two-by-two log-rank Mantel-Cox test for survival (GraphPad Prism). The computed p-values are presented in the table shown in FIG. 9D and formatted in bold font when under the threshold of 0.05. TF: Tumor Free.

FIG. 10A-FIG. 10D show individual MCA205 tumor growth curves after chemo-immunotherapeutic treatment in mice auto-immunized against ACBP/DBI. For means±SEM see FIG. 1B. FIG. 10A shows tumor volume (mm³) of administration of KLH-DBI as compared to KLH alone. FIG. 10B shows tumor volume (mm³) after administration of KLH+OXA+αPD1 as compared to KLH alone. FIG. 10C shows tumor volume (mm³) after administration of KLH-DBI+OXA+αPD1 as compared to KLH-DBI. FIG. 10D shows tumor volume (mm³) after administration of KLH-DBI+OXA+αPD1 as compared to KLH+OXA+αPD1.

FIG. 11A-FIG. 11D show individual MCA205 tumor growth curves after chemo-immunotherapeutic treatment and ACBP/DBI neutralization. For means±SEM see FIG. 2B. FIG. 11A shows tumor volume (mm³) after administration of αDBI as compared to a control (CTR). FIG. 11B shows tumor volume (mm³) after administration of OXA+αPD1 as compared to CTR. FIG. 11C shows tumor volume (mm³) after administration of αDBI+OXA+αPD1 as compared to αDBI. FIG. 11D shows tumor volume (mm³) after administration of αDBI+OXA+αPD1 as compared to OXA+αPD1.

FIG. 12A-FIG. 12D show individual TC1 tumor growth curves after chemo-immunotherapeutic treatment and ACBP/DBI neutralization. For means±SEM see FIG. 5A. FIG. 12A shows total flux (p/s) after administration of αDBI as compared to a control (CTR). FIG. 12B shows total flux (p/s) after administration of OXA+αPD1 as compared to CTR. FIG. 12C shows total flux (p/s) after administration of αDBI+OXA+αPD1 as compared to αDBI. FIG. 12D shows total flux (p/s) after administration of αDBI+OXA+αPD1 as compared to OXA+αPD1.

FIG. 13A-FIG. 13D show individual TC1 tumor growth curves after chemo-immunotherapeutic treatment and ACBP/DBI neutralization in the context of corticotherapy. For means±SEM see FIG. 7A. FIG. 13A shows total flux (p/s) after administration of αPD1+OXA as compared to a control (CTR). FIG. 13B shows total flux (p/s) after administration of CORT+αPD1+OXA as compared to αPD1+OXA. FIG. 13C shows total flux (p/s) after administration of CORT+αPD1+OXA+αDBI as compared to CORT+αPD1+OXA. FIG. 13D shows total flux (p/s) after administration of CORT+αPD1+OXA+αDBI as compared to αPD1+OXA+αDBI.

FIG. 14A-FIG. 14D show individual E0771 tumor growth curves after immunotherapy treatment and ACBP/DBI neutralization in the context of corticotherapy. For means±SEM see FIG. 8B. FIG. 14A shows tumor area (mm²) after administration of αPD1 as compared to a control (CTR). FIG. 14B shows tumor area (mm²) after administration of CORT+αPD1 as compared to αPD1. FIG. 14C shows tumor area (mm²) after administration of CORT+αPD1+αDBI as compared to CORT+αPD1. FIG. 14D shows tumor area (mm²) after administration of CORT+αPD1+αDBI as compared to αPD1+αDBI.

FIG. 15A-FIG. 15D show individual MCA205 tumor growth curves after vaccination and ACBP/DBI neutralization in the context of corticotherapy. For means±SEM see FIG. 9B. FIG. 15A shows tumor area (mm²) after administration of mitoxantrone (MTX) as compared to a control (CTR). FIG. 15B shows tumor area (mm²) after administration of CORT+MTX as compared to MTX alone. FIG. 15C shows tumor area (mm²) after administration of CORT+MTX+αDBI as compared to CORT+MTX. FIG. 15D shows tumor area (mm²) after administration of CORT+MTX+αDBI as compared to MTX+αDBI.

FIG. 16A-FIG. 16O: Overabundance of DBI (ACBP) in HCC patients based on an analysis of data from The Tumor Cell Genomic Atlas (TCGA) dealing with liver hepatocellular carcinoma (HCC or LIHC). FIG. 16A show higher expression of DBI mRNA in HCC tissues versus normal tissues. FIG. 16B and FIG. 16C shows that DBI mRNA levels are increased in HCC tissues compared with normal tissues independent of tumor grades and TNM stages in TCGA. FIG. 16D shows elevated DBI mRNA levels are detected in tumor tissues in HCC patients with an AFP>400 ng/ml compared with AFP≤400 ng/ml. FIG. 16E show that high DBI expression is associated with poor prognosis of HCC patients FIG. 16F-FIG. 16L. shows an analysis of data from the AP-HP cohort. FIG. 16F show that DBI plasma levels are increased in HCC patients compared to controls. FIG. 16G-FIG. 16I show that elevated DBI plasma levels are associated with advanced BCLC stages, vascular invasion, and tumor metastasis. FIG. 16J-FIG. 16K shows a positive correlation between DBI and AFP both at mRNA and protein levels. FIG. 16L show that plasma DBI levels are positively associated with largest tumor size of HCC patients. FIG. 16M-FIG. 16O shows tumor origin of DBI in mice bearing human HCC. FIG. 16M is a schematic diagram of orthotopic liver transplantation of HUH-7 cells in nude mice. FIG. 16N shows detection of human DBI in mouse plasma after tumor implantation by ELISA. FIG. 16O shows HUH-7 cell-derived human DBI in mouse plasma correlates with tumor size.

FIG. 17A-FIG. 17M: Knockdown of DBI/Dbi attenuates proliferation, reduces clonogenic potential and arrests the cell cycle of liver cancer cells. FIG. 17A shows DBI mRNA and protein expression profiles in different liver cancer cell lines. FIG. 17B-FIG. 17D show DBI/Dbi depletion efficiency verified by qRT-PCR in single cell clones, derived from distinct parental cell lines including HUH-7, HEP-G2 and Hep55.1C. FIG. 17E-FIG. 17G show a CCK-8 proliferation assay of HUH-7, HEP-G2 and Hep55.1C-derived DBI/Dbi knockdown and control cell lines. FIG. 17H-FIG. 17J are representative images and quantifications of colony formation assays and FIG. 17K-FIG. 17M show cytofluorometric analysis of cell cycle distributions of the aforementioned liver cancer cell lines.

FIG. 18A-FIG. 18Q: Intra- and extracellular DBI inhibition blunt tumor development in an orthotopic transplantation model. FIG. 18A is a schematic diagram of the orthotopic HCC model involving intrahepatic injection of Hep55.1C cell-derived DBI depletion clones (SH1, SH2, and SH3) or parental control clones (NC). FIG. 18B-FIG. 18E show a log-rank test for survival curve, tumor incidence, number of HCC nodules, and tumor weight of mice at endpoint (n=19-23 animals per group). FIG. 18F is a schematic diagram of the orthotopic HCC model using Hep55.1C-Luc derived cell clones (NC, SH1, SH2, and SH3). FIG. 18G shows survival of mice treated as showed in FIG. 18F. FIG. 18H shows representative IVIS images of HCC lesions for the different experimental conditions. FIG. 18I shows quantification of total intensity of tumor lesions from IVIS images. FIG. 18J-FIG. 18K show number of HCC nodules, and tumor weight of mice at endpoint (n=18-19 mice per group). FIG. 18L is a schematic diagram of the orthotopic HCC mouse model (Hep55.1C-Luc) treated with KLH/KLH-DBI. FIG. 18M shows survival of mice treated as shown in FIG. 18L. FIG. 18N-FIG. 18O show representative IVIS images and quantification of tumor burden by IVIS imaging. FIG. 18P-FIG. 18Q show of HCC codules and tumor weight of mice at endpoint (n=15-21 mice per group).

FIG. 19A-FIG. 19L: Intra- and extra-cellular DBI inhibition delay hepatic oncogenesis driven by Myc plus Ctnnb1. FIG. 19A is a schematic diagram of Myc/Ctnnb1-induced hepatic carcinogenesis in tamoxifen-induced conditional Dbi knockout mice (Dbi^{-/-}) and control mice (Dbi^{+/+}). FIG. 19E is a s\Schematic diagram of Myc/Ctnnb1-induced hepatocarcinogenesis in Gabrg2-mutated (Gabrg2^{F77I/F77I}) and control (Gabrg2^{WT}) mice. FIG. 19I is a schematic diagram of Myc/Ctnnb1-induced hepatic tumorigenesis in KLH/KLH-DBI (ACBP) immunized mice. FIG. 19B, FIG. 19F, and FIG. 19J show survival of mice in days. FIG. 19C, FIG. 19G, and FIG. 19K show sizes of the largest tumor per mouse. FIG. 19D, FIG. 19H, and FIG. 19L show quantification of the number of tumors with different sizes (<2 mm, 2-5 mm, 5-10) mm, and >10 mm).

FIG. 20A-FIG. 20X: Intra- and extracellular DBI inhibition impaired NASH-driven hepatocarcinogenesis. FIG. 20A is a schematic diagram of hepatic carcinogenesis induced by Western-style diet (WD) plus CCl₄ in tamoxifen-induced conditional Dbi knockout mice (Dbi^{-/-}) and control mice (Dbi^{+/+}). FIG. 20G is a schematic diagram of WD plus CCl₄-induced hepatic carcinogenesis in Gabrg2-mutated (Gabrg2^{F77I/F77I}) and control (Gabrg2^{WT}) mice. FIG. 20M is a schematic diagram of WD plus CCl₄-induced hepatic carcinogenesis in KLH/KLH-ACBP immunized mice. FIG. 20S is a schematic diagram of HFD plus DEN-induced hepatic carcinogenesis in KLH/KLH-DBI immunized mice. FIG. 20B, FIG. 20H, FIG. 20N, and FIG. 20T show representative immunoblots detecting DBI protein in the liver. GAPDH was used as a loading control. FIG. 20C, FIG. 20I, FIG. 20O, and FIG. 20U show quantification of Western blots. Densitometric ratios of DBI/GAPDH were normalized to control groups, shown as (Nor.). FIG. 20D, FIG. 20J, FIG. 20P, and FIG. 20V show plasma DBI levels. Values were normalized to control groups, presented as (Nor.). FIG. 20E, FIG. 20K, FIG. 20Q, and FIG. 20W show qQuantification of the largest tumor size of each treatment group. FIG. 20F, FIG. 20L, FIG. 20R, and FIG. 20X show quantification of the number of tumors with different sizes. n=5-27 per group.

FIG. 21A-FIG. 21F: DBI neutralization enhances the susceptibility of HCC to immunotherapy. FIG. 21A is a cross-species comparison of dysregulated molecular pathways between three mouse models and human liver diseases. FIG. 21B shows representative KEGG pathways in three mouse datasets generated in this study. FIG. 21C is a schematic diagram of anti-DBI (ACBP) plus anti-PD1 combination therapy tested in orthotopic Hep55.1C HCC model. FIG. 21D shows survival of the mice in days. FIG. 21E-FIG. 21F show tumor weight and number of tumor nodules per mouse in each treatment group (n=9) per group).

FIG. 22A-FIG. 22E: Transcriptomic signatures of DBI inhibition in NASH-driven HCC models. FIG. 22A is a heatmap of the gene expression profiles in three NASH-driven HCC mouse models. FIG. 22B shows commonly up- or downregulated genes in three mouse models. FIG. 22C is a flow diagram summarizing the strategy to identify gene set O1. Gene set O1 was defined as genes downregulated by DBI inhibition in ≥2 mouse NASH models that are also overexpressed in human HCC, as well as associated with poor prognosis in TCGA-LIHC. FIG. 22D is a heatmap of gene set O1 in FIG. 22C. FIG. 22E shows gene ontology (GO) and KEGG pathway analysis of gene set O1. Enriched GO-terms for biological process (BP), molecular function (MF), and cellular component (CC) are shown.

FIG. 23A-FIG. 23B: DBI inhibition regulates cell cycle-relevant genes. FIG. 23A shows that DBI inhibition downregulated genes that were positive regulators of cell cycle (Ccnd1, Cdk4, Cdk6, Ccne1 or Pena) and upregulated genes that inhibit cell cycle progression (Atr, Gadd45a, Gadd45b, Cdkn1a or Cdkn2a) in four NASH-driven HCC models. FIG. 23B is a heatmap showing the expression profile of cell cycle-related genes in HEP-G2-derived DBI knockdown cell lines.

FIG. 24A-FIG. 24I: DBI inhibition decreased cell proliferation. FIG. 24A-FIG. 24E are representative images and quantification of Ki67 IHC staining in liver sections from distinct mouse models (n=3-10) samples per treatment group). T/NT represents Tumor/Non-tumor. FIG. 24F is a schematic diagram of the strategy to generate primary Dbi^{+/+} and Dbi^{-/-} HCC cells in the WD+CCl₄-induced mouse model. FIG. 24G shows representative images of DBI (ACBP), Ki67, PCNA, CK19, AFP and GPC3 immunofluorescence staining in primary Dbi^{+/+} and Dbi^{-/-} HCC cells and quantification. FIG. 24H shows representative images and quantification of colony formation of primary Dbi^{+/+} and Dbi^{-/-} HCC cells. FIG. 24I shows a CCK-8 proliferation assay in HCC primary cells.

FIG. 25A-FIG. 25C: DBI inhibition enhances ferroptosis sensitivity at the transcriptional level. FIG. 25A is a qRT-PCR analysis showing upregulation of ferroptosis-promoting genes and downregulation of ferroptosis-inhibiting genes by DBI inhibition. Results were displayed in heatmaps (n=5-10) samples per group). FIG. 25B is a quantification of western blots showing ferroptosis and autophagy-related proteins after inhibition of DBI. FIG. 25C shows the spatial transcriptomic landscape of liver sections from WD/CCl₄-induced HCC models with Dbi^{+/+}/Dbi^{-/-} mice or KLH/KLH-DBI immunized mice. Black rectangles indicate regions of interest for each sample.

FIG. 26A-FIG. 26P: DBI neutralization increased sensitivity to ferroptosis induction in vitro and in vivo. FIG. 26A-FIG. 26D show CCK-8 viability assays of Dbi^{+/+} and Dbi^{-/-} HCC cells treated with ferroptosis inducers, including RSL3, IKE (imidazole ketone erastin), LA (linoleic acid), and LNA (linolenic acid). FIG. 26E and FIG. 26K are schematic diagrams of anti-DBI plus RSL3 or IKE combination therapy tested in orthotopic Hep55.1C-Luc HCC model. FIG. 26F and FIG. 26L show survival of the mice in days. FIG. 26G, FIG. 26H, FIG. 26M, and FIG. 26N are representative IVIS images of HCC lesions and quantification of total intensity of HCC lesions from IVIS images at day 25/23, respectively. FIG. 26I, FIG. 26J, FIG. 26O, and FIG. 26P show tumor weight and number of HCC nodules of mice at the human endpoint (n=7-9) animals per group).

### DETAILED DESCRIPTION

Unless otherwise defined, all technical and scientific terms used herein have the same meaning as commonly understood by those of ordinary skill in the art to which the present disclosure relates. Any methods, systems, and materials similar or equivalent to those described herein can be used in the practice of embodiments described herein. The following definitions are provided to facilitate understanding of certain terms used frequently herein and are not meant to limit the scope of the present disclosure.

### Definitions

As used herein, the term “administering” refers to the physical introduction of a composition comprising a therapeutic agent to a subject, using any of the various methods and delivery systems known to those skilled in the art. In some instances, routes of administration for the agents described herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include a topical, epidermal, oral or mucosal route of administration, for example, intranasally, vaginally, rectally, sublingually or topically. Administering can be performed, for example, once, a plurality of times, and/or over one or more extended periods.

As used herein the term “agent” or grammatical equivalents thereof refers to chemical or biological entities, such as antibodies, antigen binding portions thereof, fragments thereof, aptamers, compounds, small molecules, drugs, etc., or an active portion thereof, that are capable of eliciting a biological action on a biological target of interest, such as anti-DBI agents, anti-cancer agents, corticotherapy agents, etc.

As used herein the term “antibody” refers to immunoglobulin molecules and immunologically active portions of immunoglobulin molecules, i.e., molecules that contain an antigen binding site that immune-specifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD. IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. Also encompassed by the term antibody, are heavy-chain antibodies such as camelid antibodies that contain only two heavy chains and lack the two light chains usually found in other mammalian antibodies. Antibodies described herein also include single-domain antibody (sdAb), also known as a nanobody, which is an antibody fragment consisting of a single monomeric variable antibody domain, for instance a VHH which is the antigen binding fragment of a heavy-chain antibody. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

The term “antigen” as used herein refers to any known or unknown substance that can be recognized by an antibody, including proteins, glycoproteins and carbohydrates. In some embodiments, these antigens include biologically active proteins, such as hormones, cytokines and their cell surface receptors, bacterial or parasitic cell membranes or purified components thereof, and viral antigen. In one example, the antigens expressed on the surface of said cells are antigens which are difficult to purify or antigens which lose desired epitopes upon biotinylation such as those antigens described above. In another example, the antigen is unknown and the antigen is any material that would provide a source of possible antigens. In some embodiments, that material is of animal origin, e.g., mammalian, plant, yeast, bacterial or viral origin. The material may be a cell or a population of cells for which it would be desirable to isolate antibodies, such as mammalian cells, immunomodulatory cells, lymphocytes, monocytes, polymorphs. T cells, cancer cells, tumor cells, yeast cells, bacterial cells, infectious agents, parasites and plant cells. In some embodiments, the cell is a tumor cell.

The term “aptamer” as used herein refers to a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods (Colas et al., 1996).

The term “anti-cancer agent” as used herein refers to any substance used in the treatment of hyperproliferative diseases such as cancer, e.g., in mammals such as humans. Examples of anti-cancer agents are disclosed herein.

The term “B cell” as used herein includes any B cell or derivative thereof producing an antibody, such as a B-lymphocyte, a plasma cell, a plasmablast, an activated B cell or a memory B cell. These cells may secrete antibodies and/or maintain antibodies on the surface of the cell. The population of B cells for use in the present disclosure will be any population suspected of containing at least one B cell capable of producing antibodies having the desired function. B cells for use in the present disclosure may be obtained from various sources. For example, B cells can be obtained from an animal which has either been immunized with an antigen, or which has developed an immune response to an antigen as a result of disease. Alternatively, B cells can, for example, be obtained from an immunized naïve animal which has not previously been exposed to the antigen of interest (or an animal which is not known to have been exposed to the antigen of interest or which is not believed to have been exposed to the antigen of interest).

The term “binding” as used herein refers to an association between two molecules, due to, for example, covalent, electrostatic, hydrophobic, ionic and/or hydrogen-bond interactions, including interactions such as salt bridges and water bridges. As used herein, the term “binding” in the context of the binding of an antibody to a predetermined target molecule (e.g., an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “cancer” has its general meaning in the art and includes, but is not limited to, solid tumors and blood borne tumors. The term cancer includes diseases of the skin, tissues, organs, bone, cartilage, blood, and vessels. The term “cancer” further encompasses both primary and metastatic cancers. Examples of cancers that may treated by methods and therapeutic regimens of the disclosure include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, carcinoma, undifferentiated carcinoma, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, ceruminous sebaceous adenocarcinoma, adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, mammary Paget's disease, acinar cell carcinoma, adenosquamous carcinoma, adenocarcinoma w/squamous metaplasia, thymoma, ovarian stromal tumor, thecoma, granulosa cell tumor, roblastoma, Sertoli cell carcinoma, leydig cell tumor, lipid cell tumor, paraganglioma, extra-mammary paraganglioma, pheochromocytoma, glomangiosarcoma, melanoma, amelanotic melanoma, superficial spreading melanoma, melanoma in giant pigmented nevus, epithelioid cell melanoma, blue nevus, sarcoma, fibrosarcoma, fibrous histiocytoma, myxosarcoma, liposarcoma, leiomyosarcoma, rhabdomyosarcoma, embryonal rhabdomyosarcoma, alveolar rhabdomyosarcoma, stromal sarcoma, mixed tumor, mullerian mixed tumor, nephroblastoma, hepatoblastoma, carcinosarcoma, mesenchymoma, brenner tumor, phyllodes tumor, synovial sarcoma, mesothelioma, dysgerminoma, embryonal carcinoma, teratoma, struma ovarii, choriocarcinoma, mesonephroma, hemangiosarcoma, hemangioendothelioma, kaposi's sarcoma, hemangiopericytoma, lymphangiosarcoma, osteosarcoma, juxtacortical osteosarcoma, chondrosarcoma, chondroblastoma, mesenchymal chondrosarcoma, giant cell tumor of bone, ewing's sarcoma, odontogenic tumor, ameloblastic odontosarcoma, ameloblastoma, ameloblastic fibrosarcoma, pinealoma, chordoma, glioma, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, neurofibrosarcoma, neurilemmoma, granular cell tumor, lymphoma, Hodgkin's disease, Hodgkin's lymphoma, paragranuloma, lymphoma, small lymphocytic, large cell, diffuse lymphoma, follicular lymphoma, mycosis fungoides, other specified non-Hodgkin's lymphomas, histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia. In some embodiments, the cancer is a malignant cancer.

As used herein, the term “co-administration” can refer to any of the following: (i) combining two or more agents together and administering them at a single time, (ii) administering a first agent and then administering a second agent a short time later (e.g., 1, 2, 5, 10, 15, 20, 30, and 45 min.; and 1, 2, 4, 6, 8, 16, and 24 hours later), (iii) administering an agent to a subject already undergoing long-term treatment with a first agent and/or with a second agent, (iv) administering two or more agents simultaneously each by a different route of administration. For example, a subject suffering from cancer can be administered with a single dose of a mixture of an anti-cancer agent described herein and an anti-DBI agent described herein. In another example, a subject can be administered an anti-cancer agent via intravenous infusion and simultaneously administered parentally with an anti-DBI agent described herein. All these co-administrations can be performed over the course of an anti-cancer therapy cycle, such as a chemotherapy cycle, e.g., 3 times per week for 3 weeks. Additionally, a subject can be administered parentally with an anti-DBI agent composition for a period of time (e.g., 1, 2, 7, and 14 days) preceding the administration of an anti-cancer agent. In some embodiments, a cancer patient can be administered an anti-DBI agent composition each day during a standard course of chemotherapy. Thus, “co-administration” encompasses the simultaneous, separate or sequential administration of two or more agents.

The terms “conjugated.” “joined.” “linked” and grammatical equivalents thereof as used herein, refers to any manner in which two components are coupled to each other including, but not limited to, chemically (either covalent or non-covalent that proximally associates one molecule with second molecule or one component with a second component), electrostatically or through the creation of fusion constructs of a ligand and therapeutic agent by employing all tools of molecular biology.

As used herein, the term “DBI” has its general meaning in the art and refers to the diazepam binding inhibitor, or acyl-CoA binding protein encoded by the DBI gene (Gene ID: 1622). In some embodiments, DBI refers to extracellular DBI. The term is also known as EP; ACBP; ACBD1; and CCK-RP. An exemplary amino acid sequence for DBI is represented by the NCNI reference sequence NP_001073331.1 (SEQ ID NO:1) (acyl-CoA-binding protein isoform 1). An exemplary human nucleic acid sequence is represented by the NCNI reference sequence NM_001079862.2 (SEQ ID NO:2) (acyl-CoA-binding protein isoform 1).

SEQ ID NO: 1
MSQAEFEKAA EEVRHLKTKP SDEEMLFIYG HYKQATVGDI
NTERPGMLDF TGKAKWDAWN ELKGTSKEDA MKAYINKVEE
LKKKYGI
SEQ ID NO: 2
GCTCGCCCGA GCAGGGTTGG GGCGAGTGGA CCGCGCCTCT
AAAGGCGCTT GCCAGTGCAA TCTGGGCGAT CGCTTCCTGG
TCCTCGCCTC CTCCGCTGTC TCCCTGGAGT TCTTGCAAGT
CGGCCAGGAT GTCTCAGGCT GAGTTTGAGA AAGCTGCAGA
GGAGGTTAGG CACCTTAAGA CCAAGCCATC GGATGAGGAG
ATGCTGTTCA TCTATGGCCA CTACAAACAA GCAACTGTGG
GCGACATAAA TACAGAACGG CCCGGGATGT TGGACTTCAC
GGGCAAGGCC AAGTGGGATG CCTGGAATGA GCTGAAAGGG
ACTTCCAAGG AAGATGCCAT GAAAGCTTAC ATCAACAAAG
TAGAAGAGCT AAAGAAAAAA TACGGGATAT GAGAGACTGG
ATTTGGTTAC TGTGCCATGT GTTTATCCTA AACTGAGACA
ATGCCTTGTT TTTTTCTAAT ACCGTGGATG GTGGGAATTC
GGGAAAATAA CCAGTTAAAC CAGCTACTCA AGGCTGCTCA
CCATACGGCT CTAACAGATT AGGGGCTAAA ACGATTACTG
ACTTTCCTTG AGTAGTTTTT ATCTGAAATC AATTAAAAGT
GTATTTGTTA CTTTAAATAA CTTTAAAAAA AAAA

As used herein, the term “DBI activity” refers to any biological activity of DBI that includes among others: inhibition of autophagy, induction of hypoglycaemia, stimulation of food intake, stimulation of weight gain, reduction of fatty acid oxidation, upregulation of glucose transporter, upregulation of PPARG, stimulation of glucose uptake, stimulation of glycolysis or stimulation of lipogenesis, or any combinations thereof.

As used herein, the terms “encodes” or “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the terms “enhance,” “increase,” “augment,” “improve,” and grammatical equivalents thereof when in reference to the level of any molecule (e.g., amino acid sequence, and nucleic acid sequence, antibody, etc.), cell (e.g., B cell, T cell, tumor cell), and/or phenomenon (e.g., disease treatment), in a first sample (or in a first subject) relative to a second sample (or relative to a second subject or control), mean that the quantity of molecule, cell and/or phenomenon in the first sample (or in the first subject) is higher than in the second sample (or in the second subject or control) by any amount that is statistically significant using any art-accepted statistical method of analysis. For example, it may refer to a natural, synthetic or engineered compound, agent, or component that has a biological effect to increase the efficacy of an immune checkpoint, an anti-cancer treatment, an immunotherapy treatment, or any combinations thereof.

As used herein, the term “immune checkpoint inhibitors” or “ICI” refers to agents that target and inhibit immune checkpoints, such as PD1 (programmed death 1), PDL1 (programmed cell death-ligand 1), CTLA4 (cytotoxic T-lymphocyte-associated protein 4), PDL2 (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG, and in some cases targeting so-called intrinsic checkpoint blockades, for instance molecules with ubiquitin ligase activity, such as CBLB (Casitas b-lineage lymphoma Proto-Oncogene B) and CISH (Cytokine Inducible SH2 Containing Protein). Inhibitors described herein include agents, such as antibodies or chemical molecules that target immune checkpoints, such as for PD-1, CTLA4, TIGIT, PD-L1, PDL2, KIR, B7-H3, B7-H4, BTLA, LAG3, TIM-3. VISTA, ILT2/LILRB1, ILT3/LILRB4, ILT4/LILRB2, TIGIT, NKG2A, PVRIG, CBLB, CISH, or any combinations thereof. The term “Programmed Death-1” or “PD1” refers to an immunoinhibitory receptor belonging to the CD28 family. PD1 is expressed predominantly on previously activated T cells in vivo, and binds to two ligands, PDL1 and PDL2. The term “PD1” as used herein includes human PD-1 (hPD1), variants, isoforms, and species homologs of hPD1, and analogues having at least one common epitope with hPD1. The complete hPD1 sequence can be found under GenBank Accession No. U64863. A PD1 antagonist or an anti-PD1 agent refers to any agent which blocks the inhibitory effect of PD1 on the immune system. For example, a PD1 antagonist includes agents or moieties, which directly block the binding of PD1 to its receptor, and agents or moieties, which have an allosteric effect on the activity of PD1. The term “Programmed Death Ligand-1” or “PD-L1” is one of two cell surface glycoprotein ligands for PD1 (the other being PDL2) that down-regulate T cell activation and cytokine secretion upon binding to PD1. The term “PDL1” as used herein includes human PDL1 (hPDL1), variants, isoforms, and species homologs of hPDL1, and analogues having at least one common epitope with hPDL1. The complete hPDL1 sequence can be found under GenBank Accession No. Q9NZQ7.

As used herein, the term “immunogenic cell death” or “ICD” refers to a form of cell death caused by some cytostatic agents such as oxaliplatin, cyclophosphamide, and mitoxantrone (Galluzzi et al., Cancer Cell. 2015 Dec. 14; 28(6):690-714) and anthracyclines, bortezomib, radiotherapy and photodynamic therapy (PDT) (Garg et al. (2010) “Immunogenic cell death, DAMPs and anti-cancer therapeutics: an emerging amalgamation” Biochim Biophys Acta, 1805 (1): 53-71). Unlike normal apoptosis, which is mostly nonimmunogenic or even tolerogenic, immunogenic apoptosis of cancer cells can induce an effective anti-tumor immune response through activation of dendritic cells (DCs) and consequent activation of specific T cell response. ICD is characterized by secretion of damage-associated molecular patterns (DAMPs).

The term “immunotherapy” refers to any treatment whose mechanism of action, in part or predominantly, acts via enhancing an individual's immune response.

The term “immunotherapy agent” as used herein refers to any molecule, compound, or agent that elicits an active immune response. In some instances, the immunotherapeutic agent activates the immune system. In some instances, the immunotherapeutic agent is a cancer-targeted immunotherapeutic agent. In some instances, the immunotherapeutic agent elicits an active immune response against cancer or cells thereof. In some instances, the immunotherapeutic agent elicits a general immune response. In some instances, the immunotherapeutic agent elicits an immune response against a specific protein, protein group, transcript or group of transcripts. For example, immunotherapy agents can comprise immune checkpoint inhibitors, chimeric antigen receptor CAR-T cells, CAR-NK cells cytotoxic monoclonal antibodies, and vaccines. Examples of immunotherapy agents are disclosed herein.

As used herein, the terms “inhibit,” “reduce,” “suppress,” “decrease,” “neutralize,” and grammatical equivalents when in reference to the level of any molecule (e.g., amino acid sequence, and nucleic acid sequence, antibody, etc.), cell (e.g., B cell, T cell, tumor cell), and/or phenomenon (e.g., disease symptom), in a first sample (or in a first subject) relative to a second sample (or relative to a second subject or control), mean that the quantity of molecule, cell and/or phenomenon in the first sample (or in the first subject) is lower than in the second sample (or in the second subject or control) by any amount that is statistically significant using any art-accepted statistical method of analysis. For example, it may refer to a natural, synthetic or engineered compound, agent, or component that has a biological effect to inhibit a protein such as extracellular DBI.

As used herein, the terms “monoclonal antibody,” “monoclonal Ab,” “monoclonal antibody composition,” “mAb,”, and the like, refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts. Examples of such compounds are disclosed herein.

As used herein, the term “neutralizing anti-DBI monoclonal antibody” refers to an antibody or a monoclonal antibody having specificity for DBI and that inhibits, reduces or completely neutralizes the activity of DBI (for example, extracellular DBI). Whether an antibody is a neutralizing antibody can be determined by in vitro assays, such as any described in the examples. Typically, the neutralizing antibody of the present disclosure inhibits the activity of extracellular DBI by at least 50%, 60%, 70%, 80%, 90%, 95%, 99%, or 100%. Examples of such antibodies are disclosed herein.

As used herein, the term “protein” refers to polymers of amino acids of any length. The terms also encompass an amino acid polymer that has been modified; for example, by disulfide bond formation, glycosylation, lipidation, phosphorylation, or conjugation with a labeling component.

As used herein, the term “refractory cancer” refers to a cancer than does not respond to treatment. Some cancer cells have ways of defending themselves against chemotherapy drugs, immunotherapy drugs, biological agents and/or radiation therapy. In such cases, the cancer is termed refractory.

The term, “separate” administration as used herein means the administration of each of two or more compounds/agents to a subject from non-fixed dose dosage forms simultaneously, substantially concurrently, or sequentially in any order. There may, or may not, be a specified time interval for administration of each the compounds.

The term “sequential” administration as used herein means the administration of each of two or more compounds/agents to a subject from non-fixed (separate) dosage forms in separate actions. The administration actions may, or may not, be linked by a specified time interval. For example, administering compounds over a specified time such as once every 14 to 21 days.

As used herein, the term “specific” or “specificity” refers to the ability of an antibody to detectably bind target molecule (e.g., an epitope presented on an antigen) while having relatively little detectable reactivity with other target molecules. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

As used herein, the terms “subject” and “patient” is used interchangeably and refers to any subject for whom diagnosis, treatment, or therapy is desired or has been administered, such as humans. Other subjects may include cattle, dogs, cats, guinea pigs, rabbits, rats, mice, horses, and the like. In some embodiments the subject is a human.

As used herein, the term “therapeutically effective amount” refers to a sufficient amount of one or more of agents of the present disclosure for reaching a therapeutic effect. It will be understood, however, that the total daily usage of the compounds/agents and compositions of the present disclosure will be decided by the attending physician within the scope of sound medical judgment. The specific therapeutically effective dose level for any particular subject will depend upon a variety of factors including the disorder being treated and the severity of the disorder; activity of the specific compound/agent employed; the specific composition employed, the age, body weight, general health, sex and diet of the subject; the time of administration, route of administration, and rate of excretion of the specific compound/agent employed; the duration of the treatment; drugs used in combination or coincidental with the specific compound/agent employed; and like factors well known in the medical arts. For example, it is well within the skill of the art to start doses of the compound/agent at levels lower than those required to achieve the desired therapeutic effect and to gradually increase the dosage until the desired effect is achieved. However, the daily dosage of the products may be varied over a wide range from 0.01 to 4,000 mg per adult per day. In some instances, the compositions contain 0.01, 0.05, 0.1, 0.5, 1.0, 2.5, 5.0, 10.0, 15.0, 25.0, 50.0, 100, 250, 500 and 1000 mg of the active ingredient for the symptomatic adjustment of the dosage to the subject to be treated. A medicament may contain from about 0.01 mg to about 1000 mg of the active ingredient. An effective amount of the compound/agent, such as a therapeutically effective amount, may be supplied at a dosage level from 0.0002 mg/kg to about 50 mg/kg of body weight per day, especially from about 0.001 mg/kg to 10 mg/kg of body weight per day.

As used herein, the term “therapeutic regimen” refers to a pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug/agent to a subject during the initial period of a treatment regimen. An induction regimen may employ (in part or in whole) a “loading regimen,” which may include administering a greater dose of the drug/agent than a physician would employ during a maintenance regimen, administering a drug/agent more frequently than a physician would administer the drug/agent during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug/agent at a regular interval, e.g., weekly, monthly, yearly, etc.) or intermittent therapy (e.g., interrupted treatment, intermittent treatment, treatment at relapse, or treatment upon achievement of a particular predetermined criteria (e.g., pain, disease manifestation, etc.)).

As used herein, the term “treatment” or “treat” refer to both prophylactic or preventive treatment as well as curative or disease modifying treatment, including treatment of a subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

Disclosed herein is the use of an agent that stimulates autophagy by inhibiting extracellular diazepam binding inhibitor (DBI) (i.e., an anti-DBI agent), for example, in a sample, an organism, or a subject, such as a human subject. Such uses may be useful in the therapeutic treatment of cancer. The present disclosure and embodiments described herein are based, at least in part, on compelling evidence of a therapeutic effect in various tumor models upon inhibition of extracellular DBI by various methods. The embodiments arise out of a novel finding that extracellular DBI inhibition increased cancer immunosurveillance, which was demonstrated, for example, by a change in the composition of the tumor immune infiltrate showing an augmented ratio of cytotoxic T cells over regulatory T cells, as well as effects on T cell activation and exhaustion markers, and inhibited cancer progression, including inhibiting cancer cell growth, resulting in improved therapeutic prognosis. Further embodiments of the present disclosure are based on the surprising finding that extracellular DBI inhibition is able to reduce and/or reverse the immunosuppressive effects of corticotherapy and restore the therapeutic efficacy of chemotherapy, immunotherapy, or chemo-immunotherapy lost because of co-administration of corticotherapy agents. Thus, the systems and compositions described herein, and the methods of use thereof are useful for improving a therapeutic effect of immunotherapy, inducing and/or enhancing cancer immunosurveillance, inhibiting cancer progression, improving therapeutic prognosis, enhancing the therapeutic effects of anti-cancer therapy, reducing the immunosuppressive effects of corticotherapy, treating cancer, or combinations thereof.

### Anti-DBI Agents

Systems, methods and compositions described herein may comprise an agent that inhibits extracellular DBI activity or uses thereof. In some embodiments, an agent that inhibits extracellular DBI activity is an anti-DBI agent. In some embodiments, the agent that inhibits DBI activity inhibits extracellular DBI. In some embodiments, the agent that inhibits extracellular DBI activity inhibits extracellular DBI expression. In some embodiments, extracellular DBI may be mammalian DBI, such as extracellular human DBI. In some embodiments, inhibiting extracellular DBI activity thereby stimulates autophagy. In some embodiments, an agent that stimulates autophagy by inhibiting extracellular DBI activity is an anti-DBI agent. In some embodiments, an agent that stimulates autophagy by inhibiting extracellular human DBI is an anti-DBI agent. In some embodiments, the anti-DBI agent described herein is a biologic molecule or a chemical molecule. In some embodiments, a biologic molecule is an antibody, and antigen-binding portion thereof, or an aptamer. In some embodiments, chemical molecule is a small molecule, drug, or compound. In some embodiments, the anti-DBI agent described herein is an antibody, and antigen-binding portion thereof, or an aptamer directed against extracellular DBI.

In some embodiments, an antibody or aptamer described herein is directed against the fragment consisting in the amino acid sequence ranging from the amino acid residue at position 43 to the amino acid residue at position 50 in SEQ ID NO:1 (i.e., the octapeptide or OP). In some embodiments, the agent that inhibits the activity of DBI (e.g., extracellular DBI) is an aptamer directed against extracellular DBI.

In some embodiments, the agent that inhibits the activity of DBI (e.g., extracellular DBI) is an antibody directed against extracellular DBI. In some embodiments, an antibody of the present disclosure is a chimeric antibody, typically a chimeric mouse/human antibody. In some embodiments, the antibody is a humanized antibody. In some embodiments, the antibody is a human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, an antibody described herein is a neutralizing antibody. In some embodiments, the neutralizing antibody of the present disclosure does not mediate antibody-dependent cell-mediated cytotoxicity and thus does not comprise an Fc portion that induces antibody dependent cellular cytotoxicity (ADCC). In some embodiments, the neutralizing antibody does not comprise an Fc domain capable of substantially binding to a FcgRIIIA (CD16) polypeptide. In some embodiments, the neutralizing antibody lacks an Fc domain (e.g., lacks a CH2 and/or CH3 domain) or comprises an Fc domain of IgG2 or IgG4 isotype. In some embodiments, the neutralizing antibody consists of or comprises a Fab, Fab′, Fab′-SH, F(ab′) 2, Fv, a diabody, single-chain antibody fragment, or a multi-specific antibody comprising multiple different antibody fragments. In some embodiments, the neutralizing antibody is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by Idusogie et al.

Any anti-DBI antibody that inhibits the activity of DBI (e.g., extracellular DBI) is suitable for use in the methods and compositions described herein. Such anti-DBI antibodies are commercially available and described in literature, the sequences of which are known or can be derived. For instance, an antibody that inhibits the activity of DBI (e.g., extracellular DBI) and suitable for use as described herein has at least 80%, at least 85%, at least 90%, at least 95%, or has 100% sequence identity to a polypeptide sequence of an antibody selected from the group consisting of: ab231910 (Rabbit polyclonal, abcam); ab232760) (Rabbit polyclonal, abcam); ab16871 (Rabbit polyclonal, abcam); sc-30190 (Rabbit polyclonal, Santa Cruz. Biotechnology); FNab02256 (Rabbit polyclonal, Wuhan Fine Biotech Co); PA5-89139 (Rabbit polyclonal, Invitrogen); OTI4A8 (Mouse monoclonal, OriGene); OTI6E12 (Mouse monoclonal, OriGene), mAb 7A (Mouse monoclonal, Fred Hutch Antibody Technology); Abcam (catalogue no. ab16871; RRID: AB_302557); DBI human or mouse FL-87 monoclonal antibodies from Santa Cruz (catalogue number sc-30190; RRID: AB_2211046); DBI human C-9) polyclonal antibodies from Santa Cruz (catalogue number sc-376853; RRID: AB_2722761) DBI mouse polyclonal antibodies from Abcam (catalogue number ab231910); DBI mouse 7a monoclonal antibodies from Fred Hutch Antibody Technology, DBI polyclonal antibodies from Invitrogen (catalogue numbers PA5-89139, PA5-79138, PA5-40659, PA5-102751, PA5-84066, PA5-76729 and PA5-92426); DBI monoclonal antibodies from OriGene (catalogue numbers CF813069, CF813070, CF813117, TA813069, TA81370, and TA813117); DBI polyclonal antibodies from Proteintech (catalogue number 14490-1-AP); DBI polyclonal antibodies from Abnova (catalogue number H00001622-D01P); or more than one of the foregoing.

Antibodies described herein may be obtained commercially or synthesized through any suitable method. For example, anti-DBI human monoclonal antibodies may be synthesized using peptides derived from the full length human ACBP and the phage display technology. In some embodiments, antibodies described herein are mutated antibodies. In some embodiments, antibodies described herein are selected based on favorable kinetic parameters, such as specificity for or affinity against human ACBP. Specificities of antibodies described herein can be validated by western blot, immunofluorescence and flow cytometry on human ACBP/DBI knock out cell lines. In some embodiments, antibodies described herein react only with human ACBP and not with mouse ACBP. KD measurements may also be performed where KD is the equilibrium dissociation constant (ratio of kd/ka between the antibody and its antigen) and KD and affinity are inversely related. In some embodiments, antibodies described herein have a high affinity against human ACBP. In some embodiments, antibodies described herein have favorable kinetics parameters, wherein favorable kinetic parameters are indicative of a faster association and slower dissociation. For example, Sensorgram shapes of antibodies described herein demonstrate a clear concentration-response relationship at lower concentrations.

In some embodiments, the agent that inhibits the expression of extracellular DBI is an inhibitor of expression. In some embodiments, said inhibitor of gene expression is a siRNA, an endonuclease, an antisense oligonucleotide, a thyroid hormone receptor agonist, or a ribozyme.

In some embodiments, the agent that inhibits the expression of DBI is a thyroid hormone receptor agonist. As disclosed herein, administration of a thyroid hormone receptor agonist can be used to indirectly reduce or inhibit the expression of DBI in circulation. Without wishing to be bound by theory, Applicants have discovered that administration of thyroid hormone receptor agonists (such as thyroid hormone receptor β (THR-β) agonists) to a subject results in transcriptional downregulation of Acbp/Dbi mRNA in the subject. Accordingly, thyroid hormone receptor agonists can be used to treat a disease characterized by elevated levels of DBI in a subject. Furthermore, Applicant has demonstrated that the addition of a thyroid hormone receptor agonist as described herein in combination an anti-cancer agent as described herein synergistically improves the activity of the anti-cancer agent at treating the cancer. Thus, disclosed herein are compositions for use in treating cancer in a subject, and methods of treating cancer in a subject by administering a composition, where the composition comprises an effective amount of thyroid hormone receptor agonists as described herein and an effective amount of an anti-cancer agent as described herein, wherein the effective amount of the thyroid hormone receptor agonist results in an improvement of the anti-cancer agent at treating the cancer. In some embodiments, the improvement is a reduction in the effective amount of the anti-cancer agent needed to treat the cancer, relative to an effective amount needed to treat the cancer in the absence of the effective amount of the thyroid receptor agonist. In some embodiments, the improvement is an increased in the activity of the effective amount of the anti-cancer agent at treating the cancer, relative to the activity of the same effective amount of the thyroid hormone receptor agonist at treating the cancer in the absence of the effective amount of the thyroid receptor agonist. In some embodiments, the improvement comprises enabling an anti-cancer agent, which is unable to treat the cancer in the absence of the effective amount of the agent that reduces the activity or expression of DBI, to be able to treat the cancer in the presence of the effective amount of the agent that reduces the activity or expression of DBI.

Thyroid receptor agonists as described herein that can be used to treat cancer in combination with anti-cancer agents include, for example, thyroid hormone receptor β (THR-β) agonists such as resmetirom, sobetirome, eprotirome, (2R,4S)-4-(3-chlorophenyl)-2-[(4-{[4-hydroxy-3-(propan-2-yl)phenyl]methyl}-3,5-dimethylphenoxy)methyl]-1,3,2-lambda-5-dioxaphosphinan-2-one, and (4-(3-benzyl-4-hydroxy benzyl)-3,5-dimethylphenyl)oxy)methyl)phosphonic acid. In some embodiments, the thyroid hormone receptor agonist comprises thyroid hormone receptor β (THR-β) agonists. In some embodiments, the thyroid hormone receptor agonist comprises resmetirom, sobetirome, eprotirome, (2R,4S)-4-(3-chlorophenyl)-2-[(4-{[4-hydroxy-3-(propan-2-yl)phenyl]methyl}-3,5-dimethylphenoxy)methyl]-1,3,2-lambda-5-dioxaphosphinan-2-one, or (4-(3-benzyl-4-hydroxybenzyl)-3,5-dimethylphenyl)oxy)methyl) phosphonic acid. In some embodiments, the thyroid hormone receptor agonist comprises resmetirom. In some embodiments, the thyroid hormone receptor agonist comprises sobetirome. In some embodiments, the thyroid hormone receptor agonist comprises eprotirome. In some embodiments, the thyroid hormone receptor agonist comprises (2R,4S)-4-(3-chlorophenyl)-2-[(4-{[4-hydroxy-3-(propan-2-yl)phenyl]methyl}-3,5-dimethylphenoxy)methyl]-1,3,2-lambda-5-dioxaphosphinan-2-one. In some embodiments, the thyroid hormone receptor agonist comprises or (4-(3-benzyl-4-hydroxy benzyl)-3,5-dimethylphenyl)oxy)methyl) phosphonic acid.

In some embodiments, the agent that inhibits the activity of DBI (e.g., extracellular DBI) consists of a vaccine composition suitable for eliciting neutralizing autoantibodies against extracellular DBI when administered to the subject. For the purpose of the present disclosure, the term “vaccine composition” is intended to mean a composition which can be administered to humans or to animals in order to induce an immune system response: this immune system response can result in the production of antibodies against extracellular DBI. Typically, the vaccine composition comprises at least one antigen derived from DBI. As used herein the term “antigen” refers to a molecule capable of being specifically bound by an antibody or by a T cell receptor (TCR) if processed and presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes). In some embodiments, an antigen of the present disclosure consists of a polypeptide.

In some embodiments, an antigen of the present disclosure consists of a polypeptide, wherein the polypeptide comprises an amino acid sequence having at least 80% of identity with the sequence of SEQ ID NO:1 or a fragment thereof (e.g., an epitope). In some embodiments, the polypeptide comprises (i) an amino acid sequence having at least 80% of identity with SEQ ID NO:1, or (ii) an amino acid sequence having at least 80% of identity with the amino acid sequence ranging from the amino acid residue at position 17 to the amino acid residue at position 50 in SEQ ID NO:1, or (iii) an amino acid sequence having at least 80% of identity with the amino acid sequence ranging from the amino acid residue at position 33 to the amino acid residue at position 50 in SEQ ID NO: 1, or (iv) an amino acid sequence having at least 80% of identity with the amino acid sequence ranging from the amino acid residue at position 43 to the amino acid residue at position 50 in SEQ ID NO:1.

In some embodiments, the polypeptide is conjugated to a carrier protein which is generally sufficiently foreign to elicit a strong immune response to the vaccine. Illustrative carrier proteins are inherently highly immunogenic. Both bovine serum albumin (BSA) and keyhole limpet hemocyanin (KLH) have commonly been used as carriers in the development of conjugate vaccines when experimenting with animals and are contemplated herein as carrier proteins. Proteins which have been used in the preparation of therapeutic conjugate vaccines include, but are not limited to, a number of toxins of pathogenic bacteria and their toxoids. Suitable carrier molecules are numerous and include, but are not limited to: bacterial toxins or products, for example, cholera toxin B-(CTB), diphtheria toxin, tetanus toxoid, and pertussis toxin and filamentous hemagglutinin, shiga toxin, pseudomonas exotoxin: lectins, for example, ricin-B subunit, abrin and sweet pea lectin: sub virals, for example, retrovirus nucleoprotein (retro NP), rabies ribonucleoprotein (rabies RNP), plant viruses (e.g., TMV, cow pea and cauliflower mosaic viruses), vesicular stomatitis virus-nucleocapsid protein (VSV-N), poxvirus vectors and Semliki forest virus vectors: artificial vehicles, for example, multiantigenic peptides (MAP), microspheres: Yeast virus-like particles (VLPs): malarial protein antigen; and others such as proteins and peptides as well as any modifications, derivatives or analogues of the above. Other useful carriers include those with the ability to enhance a mucosal response, such as, LTB family of bacterial toxins, retrovirus nucleoprotein (retro NP), rabies ribonucleoprotein (rabies RNP), vesicular stomatitis virus-nucleocapsid protein (VSV-N), and recombinant pox virus subunits.

### Anti-Cancer Agents

Systems, methods and compositions described herein may comprise one or more anti-cancer agents and uses thereof.

In some embodiments, the one or more anti-cancer agents comprise a small molecule, compound, a drug, an antibody, an antigen-binding portion thereof, or a fragment thereof, an aptamer, an inhibitor of expression, or the like. In some embodiments, the one or more anti-cancer agents comprise an antibody, and antigen-binding portion thereof, or a fragment thereof, an aptamer, or an inhibitor of expression. In some embodiments, the one or more anti-cancer agents comprise a small molecule, compound, drug, or antibody.

In some embodiments, the one or more anti-cancer agents described herein comprise a chemotherapy agent, an immunotherapy agent, or both. In some embodiments, the one or more anti-cancer agents comprise a chemotherapy agent, an immunotherapy agent, or both. In some embodiments, the one or more anti-cancer agents comprise a chemotherapy agent. In some embodiments, the one or more anti-cancer agents comprise an immunotherapy agent. In some embodiments, the one or more anti-cancer agents comprise a chemotherapy agent and an immunotherapy agent. In some embodiments, the anti-cancer agent comprises administration of radiation therapy to a subject with cancer.

In some embodiments, chemotherapy agents described herein are cytotoxic compounds/agents used to treat cancer. In some embodiments, the chemotherapy agent comprises: alkylating agents (such as thiotepa and cyclosphosphamide); alkyl sulfonates (such as busulfan, improsulfan, and piposulfan); aziridines (such as benzodopa, carboquone, meturedopa, and uredopa); ethylenimines; methylamelamines; altretamine; triethylenemelamine; trietylenephosphoramide; triethiylenethiophosphoramide; trimethylolomelamine; acetogenins (such as bullatacin and bullatacinone); a camptothecin (such as topotecan); bryostatin; callystatin; CC-1065 (such as its adozelesin, carzelesin, and bizelesin synthetic analogues); cryptophycins (such as cryptophycin 1 and cryptophycin 8); dolastatin; duocarmycin (such as the synthetic analogues, KW-2189 and CB1-TM1); eleutherobin; pancratistatin; a sarcodictyin; spongistatin; nitrogen mustards (such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard); nitrosureas (such as carmustine, chlorozotocin, fotemustine, lomustine, nimustine, and ranimnustine); antibiotics (such as the enedivne antibiotics, e.g., calicheamicin, calicheamicin gammall, and calicheamicin omegall); dynemicin, (such as dynemicin A); bisphosphonates (such as clodronate); esperamicin; neocarzinostatin chromophore; related chromoprotein enedivne antibiotic chromophores; aclacinomysins; actinomycin; authramycin; azaserine; bleomycins; cactinomycin; carabicin; caminomycin; carzinophilin; chromomycinis; dactinomycin; daunorubicin; detorubicin; 6-diazo-5-oxo-L-norleucine; doxorubicin (such as morpholino-doxorubicin, cyanomorpholino-doxorubicin, 2-pyrrolino-doxorubicin, and deoxy doxorubicin), epirubicin; esorubicin; idarubicin; marcellomycin; mitomycins (such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin); anti-metabolites (such as methotrexate and 5-fluorouracil (5-FU)); folic acid analogues (such as denopterin, methotrexate, pteropterin, and trimetrexate); purine analogues (such as fludarabine, 6-mercaptopurine, thiamiprine, and thioguanine); pyrimidine analogues (such as ancitabine, azacitidine, 6-azauridine, carmofur, cytarabine, dideoxyuridine, doxifluridine, enocitabine, and floxuridine); androgens (such as calusterone, dromostanolone propionate, epitiostanol, mepitiostane, and testolactone); anti-adrenals (such as aminoglutethimide, mitotane, and trilostane); folic acid replenisher (such as frolinic acid); aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids (such as maytansine and ansamitocins); mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; 2,2′,2″-trichlorotriethylamine; trichothecenes (such as T-2 toxin, verracurin A, roridin A, and anguidine); urethan; vindesine; dacarbazine; mannomustine; mitobronitol; mitolactol; pipobroman; gacytosine; arabinoside; cyclophosphamide; thiotepa; taxoids (such as paclitaxel and doxetaxel); chlorambucil; gemcitabine; 6-thioguanine; mercaptopurine; methotrexate; platinum coordination complexes (such as cisplatin, oxaliplatin, and carboplatin); vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (such as CPT-1 1); topoisomerase inhibitor RFS 2000; difluoromethylomithine (DMFO); retinoids (such as retinoic acid); capecitabine; pharmaceutically acceptable salts; acids or derivatives of any of the foregoing; or any combinations thereof.

In some embodiments, the chemotherapy agent comprises; thiotepa, cyclosphosphamide, busulfan, improsulfan, piposulfan, benzodopa, carboquone, meturedopa, bullatacin, bullatacinone, topotecan, adozelesin, carzelesin, bizelesin synthetic analogues, cryptophycin 1, cryptophycin 8, KW-2189, CB1-TM1, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, enediyne antibiotics, calicheamicin, calicheamicin gammall, calicheamicin omegall, dynemicin A, clodronate, morpholino-doxorubicin, cyanomorpholino-doxorubicin, 2-pyrrolino-doxorubicin, deoxy doxorubicin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, maytansine, ansamitocins, T-2 toxin, verracurin A, roridin A, anguidine, paclitaxel, doxetaxel, cisplatin, oxaliplatin, carboplatin, CPT-1 1, retinoic acid, pharmaceutically acceptable salts, acids or derivatives of any of the foregoing, or any combinations thereof.

In some embodiments, the chemotherapy agent comprises an immunogenic cell death (ICD) inducing activity. In some embodiments, a chemotherapy agent having an ICD activity comprises; oxaliplatin, mitoxantrone, or both.

In some embodiments, the chemotherapy agent is a small molecule, an antibody, an antigen binding portion thereof, or a fragment thereof, an aptamer, an inhibitor of expression, a small molecule, a drug, or the like. In some embodiments, chemotherapy agents can be delivered to a subject on the same or different molecules as one or more components (e.g., an immunotherapy agent, an anti-DBI agent, a corticotherapy agent, or more than one of the following, or combinations thereof) described herein. In some embodiments, chemotherapy agents can be conjugated to one or more components described herein. In some embodiments, chemotherapy agents can be fused to one or more components described herein. In some embodiments, chemotherapy agents can be covalently linked to one or more components described herein. In some embodiments, chemotherapy agents can be recombinantly synthesized onto one or more components described herein.

In some embodiments, immunotherapy agents described herein can utilize or induce polypeptides, cells and/or factors of the innate and adaptive immune system to recognize and treat cancer. In some embodiments, immunotherapy agents described herein comprise agents with activity against immune checkpoints, activated effector cells, non-specific immune-stimulatory agents (e.g., cytokines), engineered activated effector cells (e.g., chimeric antigen receptor T cells, chimeric antigen receptor NK cells), cell therapy using engineered or non-engineered tumor infiltrating lymphocytes, cancer antigen adjuvants, and the like.

In some embodiments, immunotherapy agents comprise an agent that exhibits activity against an immune checkpoint.

In some embodiments, the immune checkpoint comprises PD1 (programmed death 1), PDL1 (programmed cell death-ligand 1), CTLA4 (cytotoxic T-lymphocyte-associated protein 4), PDL2 (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG, CBLB (Casitas b-lineage lymphoma Proto-Oncogene B), CISH (Cytokine Inducible SH2 Containing Protein), or any combinations thereof.

In some embodiments, the immunotherapy agents comprise an anti-PD1 agent, anti-PD-L1 agent, anti-CTLA4 agent, anti-PD-L2 agent, anti-KIR agent, anti-B7-H3 agent, anti-B7-H4 agent, anti-BTLA agent, anti-LAG3 agent, anti-TIM-3 agent, anti-VISTA agent, anti-ILT2/LILRB1 agent, anti-ILT3/LILRB4 agent, anti-ILT4/LILRB2 agent, anti-TIGIT agent, anti-NKG2A agent, anti-PVRIG agent, anti-CBLB agent, anti-CISH agent, or any combinations thereof. In some embodiments, the immunotherapy agents comprise an anti-PD1 agent, anti-PD-L1 agent, anti-CTLA4 agent, or any combinations thereof. In some embodiments, the immunotherapy agents comprise an anti-PD1 agent. In some embodiments, the immunotherapy agents comprise an anti-PD-L1 agent. In some embodiments, the immunotherapy agents comprise an anti-CTLA4 agent. In some embodiments, the immunotherapy agent comprises an anti-PD1 agent and an anti-PD-L1 agent. In some embodiments, the immunotherapy agent comprises an anti-PD1 agent and an anti-CTLA4 agent. In some embodiments, the immunotherapy agent comprises an anti-PD-L1 agent and an anti-CTLA4 agent. In some embodiments, the immunotherapy agents comprise an anti-PD1 agent, an anti-PD-L1 agent, and anti-CTLA4 agent. In some embodiments, the immunotherapy agent is a small molecule, a biologic, an antibody, an antigen-binding portion thereof, or a fragment thereof, an aptamer, an inhibitor of expression, a small molecule, a drug, or the like.

In some embodiments, the immunotherapy agent are one or more antibodies, small molecules, or biologics that are directed against one or more immune checkpoints. In some embodiments, the one or more antibodies directed against one or more immune checkpoints comprises an anti-PD1 antibody, an anti-PD-L1 antibody, an anti-CTLA4 antibody, anti-PD-L2 antibody, anti-KIR antibody, anti-B7-H3 antibody, anti-B7-H4 antibody, anti-BTLA antibody, anti-LAG3 antibody, anti-TIM-3 antibody, anti-VISTA antibody, anti-ILT2/LILRB1 antibody, anti-ILT3/LILRB4 antibody, anti-ILT4/LILRB2 antibody, anti-TIGIT antibody, anti-NKG2A antibody, anti-PVRIG antibody, anti-CBLB antibody, anti-CISH antibody, or any combinations thereof. In some embodiments, the immunotherapy agent comprises an anti-PD1 antibody. In some embodiments, the immunotherapy agent comprises an anti-PD-L1 antibody. In some embodiments, the immunotherapy agent comprises an anti-CTLA4 antibody. In some embodiments, the immunotherapy agent comprises an anti-PD1 antibody and an anti-PD-L1 antibody. In some embodiments, the immunotherapy agent comprises an anti-PD1 antibody and an anti-CTLA4 antibody. In some embodiments, the immunotherapy agent comprises an anti-PD-L1 antibody and an anti-CTLA4 antibody, or any combinations thereof.

In some embodiments, the one or more small molecule or biologic directed against one or more immune checkpoints comprise; ipilimumab, tremelimumab, MK-1308, FPT155, PRS010, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002, CP675206, pembrolizumab, nivolumab, pidilizumab, AMP-224, BMS-936559, cemiplimab, PDR001, MDX-1105, MEDI4736, atezolizumab, avelumab, BMS-936559, durvalumab, lirlumab (IPH2102), IPH2101, MGA271, FPA150, IMP321 (eftilagimod alpha), relatlimab, MK-4280, AVA017, BI754111, ENUM006, GSK2831781, INCAGN2385, LAG3Ig, LAG525, REGN3767, Sym016, Sym022, Sym023, TSR033, TSR075, XmAb22841, LY3321367, MBG453, TSR-022, JNJ-61610588, MK-7684, PTZ-201, RG6058, COM902, IPH-2201, COM701, CA-327, LAG525, REGN3767, BI 754111, tebotelimab, FS118, MGC018, or any combinations thereof.

In some embodiments, immunotherapy agents can be delivered to a subject on the same or different molecule as one or more components (e.g., an immunotherapy agent, an anti-DBI agent, a corticotherapy agent, more than one of the following, or combinations thereof) described herein. In some embodiments, immunotherapy agents can be conjugated to one or more components described herein. In some embodiments, immunotherapy agents can be fused to one or more components described herein. In some embodiments, immunotherapy agents can be covalently linked to one or more components described herein. In some embodiments, immunotherapy agents can be recombinantly synthesized onto one or more components described herein.

### Corticotherapy Agents

Systems, methods and compositions described herein may comprise one or more corticotherapy agents and uses thereof. Corticotherapy agents described herein are anti-inflammatory corticoid active agents used to treat an inflammatory immune response to a disease.

In some embodiments, the one or more corticotherapy agents described herein comprise cortisol, cortisone, prednisone, prednisolone, methylprednisolone, dexamethasone, betamethasone, triamcinolone, deflazacort, fludrocortisone acetate, deoxycorticosterone acetate, aldosterone, beclomethasone, or any combinations thereof. In some embodiments, the corticotherapy agents comprise two or more of the foregoing. In some embodiments, the corticotherapy agents comprise three or more of the foregoing. In some embodiments, the corticotherapy agents comprise four or more of the foregoing. In some embodiments, the corticotherapy agents comprise five or more of the foregoing. In some embodiments, the corticotherapy agents comprise six or more of the foregoing. In some embodiments, the corticotherapy agents comprise seven or more of the foregoing. In some embodiments, the corticotherapy agents comprise eight or more of the foregoing. In some embodiments, the corticotherapy agents comprise nine or more of the foregoing. In some embodiments, the corticotherapy agents comprise ten or more of the foregoing.

In some embodiments, the one or more corticotherapy agents comprise cortisol. In some embodiments, the one or more corticotherapy agents comprise cortisone. In some embodiments, the one or more corticotherapy agents comprise prednisone. In some embodiments, the one or more corticotherapy agents comprise prednisolone. In some embodiments, the one or more corticotherapy agents or one or more corticotherapy moieties comprise methylprednisolone. In some embodiments, the one or more corticotherapy agents comprise dexamethasone. In some embodiments, the one or more corticotherapy agents comprise betamethasone. In some embodiments, the one or more corticotherapy agents comprise triamcinolone. In some embodiments, the one or more corticotherapy agents comprise deflazacort. In some embodiments, the one or more corticotherapy agents comprise fludrocortisone acetate. In some embodiments, the one or more corticotherapy agents comprise deoxy corticosterone acetate. In some embodiments, the one or more corticotherapy agents comprise aldosterone. In some embodiments, the one or more corticotherapy agents comprise beclomethasone.

### Systems

The present disclosure further provides for a system comprising one or more components, wherein the one or more components individually comprise one or more of (a) an agent directed against extracellular human diazepam binding inhibitor (anti-DBI agent) in an amount sufficient to inhibit extracellular DBI; (b) one or more anti-cancer agents, optionally wherein the one or more anti-cancer agents comprises a chemotherapy agent, an immunotherapy agent, or any combinations thereof; (c) one or more corticotherapy agents; or any combinations of (a)-(c). In some embodiments, the system comprises two or more components of (a) to (c). In some embodiments, the system comprises all the components of (a) to (c).

In some embodiments, the systems described herein comprise an agent directed against extracellular human diazepam binding inhibitor (anti-DBI agent) in an amount sufficient to inhibit extracellular DBI. In some embodiments, the systems described herein comprise one or more anti-cancer agents, optionally wherein the one or more anti-cancer agents comprises a chemotherapy agent, an immunotherapy agent, or any combinations thereof. In some embodiments, the systems described herein comprise one or more corticotherapy agents.

### Compositions

Disclosed herein are compositions comprising one or more of; (a) an agent directed against extracellular human diazepam binding inhibitor (anti-DBI agent) in an amount sufficient to inhibit extracellular DBI in a subject; (b) one or more anti-cancer agents, optionally wherein the one or more anti-cancer agents comprises a chemotherapy agent, an immunotherapy agent, or any combinations thereof; (c) one or more corticotherapy agents; or any combinations of (a)-(c). In some embodiments, the composition comprises two or more components of (a) to (c). In some embodiments, the composition comprises all the components of (a) to (c). In embodiments, the composition may be for use in the methods, regimens and systems disclosed herein.

In some embodiments, the compositions described herein comprise an agent directed against extracellular human diazepam binding inhibitor (anti-DBI agent) in an amount sufficient to inhibit extracellular DBI in a subject. In some embodiments, the compositions described herein comprise one or more anti-cancer agents, optionally wherein the one or more anti-cancer agents comprises a chemotherapy agent, an immunotherapy agent, or a combination thereof. In some embodiments, the compositions described herein comprise one or more corticotherapy agents.

### Pharmaceutical Compositions:

Systems, compositions, and components thereof as described herein, such as the one or more agents described herein that inhibits the activity or expression of DBI (e.g., extracellular DBI), the one or more anti-cancer agents, and/or the one or more corticotherapy agents, are administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present disclosure may be administered orally, parenterally, by inhalation spray, topically, rectally, nasally, buccally, vaginally or via an implanted reservoir. The term parental used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this disclosure may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the agent that inhibits the activity or expression of extracellular DBI is combined with emulsifying and suspending agents. If desired, certain sweetening, flavoring or coloring agents may also be added. Alternatively, the compositions of this disclosure may be administered in the form of suppositories for rectal administration. These can be prepared by mixing the one or more agents or one or more moieties with a suitable non-irritating excipient that is solid at room temperature but liquid at rectal temperature and therefore will melt in the rectum to release the drug. Such materials include cocoa butter, beeswax and polyethylene glycols. The compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active agent suspended or dissolved in one or more carriers. Carriers for topical administration of the compounds/agents/moieties of this disclosure include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active agent suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, the one or more agents that inhibit the activity or expression of DBI (e.g., extracellular DBI) of the present disclosure are administered directly into the subject or isolated organ using injection, pump device and/or any machine (e.g., bypass machine). In some embodiments, an isolated organ suitable for transplantation is perfused with a preservation solution which comprises the effective amount of the agent that inhibits the activity or expression of extracellular DBI. As used herein, the terms “preservation solution” or “organ preservation solution” refer to an aqueous solution having a pH between 6.5 and 7.5, including salts, preferably chloride, sulfate, sodium, calcium, magnesium and potassium; sugars, mannitol, raffinose, sucrose, glucose, fructose, lactobionate (which is a water resistant), or gluconate; antioxidants, for instance glutathione; active agents, for instance xanthine oxidase inhibitors such as allopurinol, lactates, amino acids such as histidine, glutamic acid (or glutamate), tryptophan; and optionally colloids such as hydroxyethyl starch, polyethylene glycol or dextran. In some embodiments, a device for preserving an organ is used wherein said device comprises an organ container filled with a preservation solution, characterized in that said device further comprises one or more mean for injecting one or more agents (e.g., the agent that inhibits the activity or expression of extracellular DBI) into the organ container.

### Methods of Treatment

Disclosed herein, amongst other disclosures are methods of improving the effect of immunotherapy, inducing immunosurveillance, inhibiting cancer progression, enhancing cancer immunosurveillance, inhibiting cancer progression, improving therapeutic prognosis, enhancing the therapeutic effect of anti-cancer therapy, reducing the immunosuppressive effect of corticotherapy, treating cancer, or any combinations thereof in a subject having cancer or in need thereof. Agents, compositions and combinations for use in such methods are also disclosed. In some embodiments, the described methods of inducing immunosurveillance, inhibiting cancer progression, improving therapeutic prognosis, enhancing the therapeutic effect of anti-cancer therapy, reducing the immunosuppressive effect of corticotherapy, or combinations thereof thereby treat a subject having cancer. In some embodiments, methods described herein are relative to a comparable method in the absence of the administration of an anti-DBI agent. In some embodiments, the absence of the administration of the anti-DBI agent is the administration of anti-cancer agent alone or the administration the corticotherapy agent alone. In some embodiments, the absence of the administration of the anti-DBI agent is the administration of the anti-cancer agent together with the corticotherapy agent.

In some embodiments, methods described herein comprise administering an agent directed against extracellular human diazepam binding inhibitor (anti-DBI agent), or composition comprising such an agent, in an amount sufficient to inhibit extracellular human DBI; one or more anti-cancer agent or composition comprising such an agent; and/or one or more corticotherapy agent or composition comprising such an agent, or any combinations thereof. In some embodiments, the one or more anti-cancer agents comprise a chemotherapy agent, an immunotherapy agent, or both.

In some embodiments, disclosed herein is a method of improving a therapeutic effect for immunotherapy in a subject as well as agents, compositions and combinations for use in such methods.

In some embodiments, the method for improving a therapeutic effect of immunotherapy in a subject comprises administering to the subject (a) an agent that stimulates autophagy by inhibiting extracellular human diazepam binding inhibitor (DBI) (anti-DBI agent) and (b) one or more immunotherapy agents. In some embodiments, the method comprises administering to the subject (a) an agent that stimulates autophagy by inhibiting extracellular human diazepam binding inhibitor (DBI) (anti-DBI agent), (b) one or more immunotherapy agents, and (c) one or more chemotherapy agents. In some embodiments, the method comprises administering to the subject (a) an agent that stimulates autophagy by inhibiting extracellular human diazepam binding inhibitor (DBI) (anti-DBI agent), (b) one or more immunotherapy agents, (c) one or more chemotherapy agents, and (d) one or more corticotherapy agents, or any combinations thereof. In some embodiments, the administration of the anti-DBI agent and the one or more immunotherapy agents is sufficient to improve the therapeutic effects of immunotherapy relative to a comparable method in the absence of the administration of the anti-DBI agent.

In some embodiments, disclosed herein is method of enhancing immunosurveillance in a subject having cancer as well as agents, compositions and combinations for use in such methods.

In some embodiments, the method of enhancing immunosurveillance enhances the therapeutic effect of an anti-cancer therapy.

In some embodiments, the method of enhancing immunosurveillance comprises administering an agent directed against extracellular human diazepam binding inhibitor (DBI) (anti-DBI agent) in an amount sufficient to inhibit extracellular human DBI. In some embodiments, the method of enhancing immunosurveillance comprises administering (a) an agent directed against extracellular human diazepam binding inhibitor (DBI) (anti-DBI agent) in an amount sufficient to inhibit extracellular human DBI; and (b) one or more anti-cancer agents. In some embodiments, the method of enhancing immunosurveillance comprises administering (a) an agent directed against extracellular human diazepam binding inhibitor (DBI) (anti-DBI agent) in an amount sufficient to inhibit extracellular human DBI; and (b) one or more corticotherapy agents. In some embodiments, the method of enhancing immunosurveillance comprises administering (a) an agent directed against extracellular human diazepam binding inhibitor (DBI) (anti-DBI agent) in an amount sufficient to inhibit extracellular human DBI; (b) one or more anti-cancer agents; and (c) one or more corticotherapy agents; or any combinations thereof.

In some embodiments, the administration of the anti-DBI agent, the one or more anti-cancer agents, and/or the one or more corticotherapy agents is sufficient to induce one or more immunosurveillance biomarkers. In some embodiments, the method of enhancing immunosurveillance results in an enhanced therapeutic effect of the anti-cancer therapy relative to a comparable method in the absence of the administration of the anti-DBI agent.

In some embodiments, the methods described herein induce at least one immunosurveillance biomarker in a sample or a subject, the at least one immunosurveillance biomarker comprising: increased CD8⁺ T cells expression; decreased CD4⁺ T cells expression; decreased T_{reg} cells (CD4⁺Foxp3⁺); an increased ratio of CD8⁺ T cells to T_{reg} cells (CD4⁺Foxp3⁺); increased T_{H}+ cells (CD4⁺Foxp3⁻); decreased expression of Lag3⁺ in T_{H}+ cells (CD4⁺Foxp3⁻); decreased expression of Lag3⁺ in CD8⁺ cells; decreased expression of Foxp3⁺ in CD4⁺ T cells; decreased T_{reg} ICOS⁺ GITR⁺ Lag3⁻ CD4⁺ T cells; or combinations thereof, as compared to the immunosurveillance biomarker incidence in the absence of the administration of the anti-DBI agent. In some embodiments, methods described herein induce at least 1, 2, 3, 4, 5, 6, 7, 8 or more immunosurveillance biomarkers.

In some embodiments, the methods described herein reduce the immunosuppressive effects of a corticotherapy relative to a comparable method in the absence of the administration of the anti-DBI agent described herein. In some embodiments, the method of reducing the immunosuppressive effects of a corticotherapy enhances the therapeutic effect of an anti-cancer therapy. In some embodiments, methods of enhancing immunosurveillance described herein induces one or more immunosurveillance biomarkers relative to a comparable method in the absence of the administration of the anti-DBI agent thereby reducing the immunosuppressive effects corticotherapy in an administered subject. Without being bound by theory it is contemplated when an anti-DBI agent described herein is administered with an anti-cancer agent and a corticotherapy agent, the anti-DBI agent induces an anti-inflammatory immune response. In some embodiments such a response promotes an antigenic response and a cytotoxic T cell environment.

In some embodiments, provided herein are methods of reducing the immunosuppressive effects of corticotherapy in subjects suffering from cancer, which comprise administering an agent directed against extracellular human diazepam binding inhibitor (DBI) (anti-DBI agent) in an amount sufficient to inhibit extracellular human DBI. In some embodiments, the method of reducing the immunosuppressive effects of corticotherapy comprises administering (a) an agent directed against extracellular human diazepam binding inhibitor (DBI) (anti-DBI agent) in an amount sufficient to inhibit extracellular human DBI; and (b) one or more anti-cancer agents. In some embodiments, the method of reducing the immunosuppressive effects of corticotherapy comprises administering (a) an agent directed against extracellular human diazepam binding inhibitor (DBI) (anti-DBI agent) in an amount sufficient to inhibit extracellular human DBI; and (b) one or more corticotherapy agents. In some embodiments, the method of reducing the immunosuppressive effects of corticotherapy comprises administering (a) an agent directed against extracellular human diazepam binding inhibitor (DBI) (anti-DBI agent) in an amount sufficient to inhibit extracellular human DBI; (b) one or more anti-cancer agents; and (c) one or more corticotherapy agents, or any combinations thereof.

In some embodiments, the reduction of the immunosuppressive effects described herein comprise one or more of; (a) increased CD8⁺ T cells expression; (b) decreased CD4⁺ T cells expression; (c) decreased T_{reg} cells (CD4⁺Foxp3⁺); (d) an increased ratio of CD8⁺ T cells to T_{reg} cells (CD4⁺Foxp3⁺); (e) increased T_{H}+ cells (CD4⁺Foxp3⁻); (f) decreased expression of Lag3⁺ in T_{H}+ cells (CD4⁺Foxp3⁻); (g) decreased expression of Lag3⁺ in CD8⁺ T cells; (h) decreased expression of Foxp3⁺ in CD4⁺ T cells; (i) decreased T_{reg} ICOS⁺ GITR⁺ Lag3⁻ CD4⁺ T cells; (j) decreased cancer amount; (k) decreased proliferation of cancer cells; (l) increased cancer cell death; (m) decreased cancer growth; (n) increased incidence of survival; (o) incidence of cancer free occurrence; (p) increased time to fatality; or any combinations of (a) to (p) in a sample relative to a comparable method in the absence of the administration of an anti-DBI agent.

In some embodiments, the reduction of the immunosuppressive effects described herein comprise any one of (a) to (p). In some embodiments, the reduction of the immunosuppressive effects described herein comprise any two or more of (a) to (p). In some embodiments, the reduction of the immunosuppressive effects described herein comprise any three or more of (a) to (p). In some embodiments, the reduction of the immunosuppressive effects described herein comprise any four or more of (a) to (p). In some embodiments, the reduction of the immunosuppressive effects described herein comprise any five or more of (a) to (p). In some embodiments, the reduction of the immunosuppressive effects described herein comprise any six or more of (a) to (p). In some embodiments, the reduction of the immunosuppressive effects described herein comprise any seven or more of (a) to (p). In some embodiments, the reduction of the immunosuppressive effects described herein comprise any eight or more of (a) to (p). In some embodiments, the reduction of the immunosuppressive effects described herein comprise any nine or more of (a) to (p). In some embodiments, the reduction of the immunosuppressive effects described herein comprise any ten or more of (a) to (p). In some embodiments, the reduction of the immunosuppressive effects described herein comprise any eleven or more of (a) to (p). In some embodiments, the reduction of the immunosuppressive effects described herein comprise any twelve or more of (a) to (p). In some embodiments, the reduction of the immunosuppressive effects described herein comprise any thirteen or more of (a) to (p). In some embodiments, the reduction of the immunosuppressive effects described herein comprise any fourteen or more of (a) to (p). In some embodiments, the reduction of the immunosuppressive effects described herein comprise any fifteen or more of (a) to (p). In some embodiments, the reduction of the immunosuppressive effects described herein comprise all sixteen of (a) to (p).

In some embodiments, disclosed herein is a method of inhibiting cancer progression in a cancer patient as well as agents, compositions and combinations for use in such methods. In some embodiments, the cancer patient is undergoing chemotherapy treatment.

In some embodiments, the method of inhibiting cancer progression comprises administering to the cancer patient an agent directed against extracellular human diazepam binding inhibitor (anti-DBI agent) in an amount sufficient to inhibit extracellular human DBI. In some embodiments, the method of inhibiting cancer progression comprises administering to the cancer patient (a) an agent directed against extracellular human diazepam binding inhibitor (anti-DBI agent) in an amount sufficient to inhibit extracellular human DBI; and (b) one or more immunotherapy agents. In some embodiments, the method of inhibiting cancer progression comprises administering to the cancer patient (a) an agent directed against a extracellular human diazepam binding inhibitor (anti-DBI agent) in an amount sufficient to inhibit extracellular human DBI; and (b) one or more corticotherapy agents. In some embodiments, the method of inhibiting cancer progression comprises administering to the cancer patient (a) an agent directed against a extracellular human diazepam binding inhibitor (anti-DBI agent) in an amount sufficient to inhibit extracellular human DBI; (b) one or more anti-cancer agents; and (c) one or more corticotherapy agents, or any combinations thereof. In some embodiments, the administration of the anti-DBI agent, one or more anti-cancer agent, and/or one or more corticotherapy agent is sufficient to inhibit one or more cancer progression biomarkers relative to a comparable method in the absence of the administration of an anti-DBI agent.

In some embodiments, the methods described herein inhibit at least one cancer progression biomarker in a subject or a sample, the at least one cancer progression biomarker comprising: a decreased cancer amount; decreased proliferation of cancer cells; increased cancer cell death; decreased cancer growth; or combinations thereof, of the subject or sample compared to the expression of the at least one cancer progression biomarker in a comparable method in the absence of the administration of the anti-DBI agent described herein. In some embodiments, methods described herein induce at least 1, 2, 3, or more cancer progression biomarker biomarkers.

In some embodiments, methods of inhibiting cancer progression inhibit at least one cancer progression biomarker in a subject, thereby improving therapeutic prognosis in the subject. In some embodiments, disclosed herein is a method of improving therapeutic prognosis in a subject.

In some embodiments, the method of improving therapeutic prognosis in a subject comprises administering an agent directed against extracellular human diazepam binding inhibitor (DBI) (anti-DBI agent) in an amount sufficient to inhibit extracellular human DBI. In some embodiments, the method of improving therapeutic prognosis in a subject comprises administering (a) an agent directed against extracellular human diazepam binding inhibitor (DBI) (anti-DBI agent) in an amount sufficient to inhibit extracellular human DBI; and (b) one or more anti-cancer agents. In some embodiments, the method of improving therapeutic prognosis in a subject comprises administering (a) an agent directed against extracellular human diazepam binding inhibitor (DBI) (anti-DBI agent) in an amount sufficient to inhibit extracellular human DBI; and (b) one or more corticotherapy agents. In some embodiments, the method of improving therapeutic prognosis in a subject comprises administering (a) an agent directed against extracellular human diazepam binding inhibitor (DBI) (anti-DBI agent) in an amount sufficient to inhibit extracellular human DBI; (b) one or more anti-cancer agents; and (c) one or more corticotherapy agents, or any combinations thereof.

In some embodiments, the improvement of the therapeutic prognosis in a subject or a sample in vitro comprises; a decreased cancer amount; decreased proliferation of cancer cells; increased cancer cell death; decreased cancer growth; or combinations thereof, of the subject or sample compared to the expression of the at least one cancer progression biomarker in a comparable method in the absence of the administration of the anti-DBI agent described herein.

In some embodiments, disclosed herein is a method of treating cancer in a subject in need thereof as well as agents, compositions and combinations for use in such methods. In some embodiments, the method comprises administering to the subject an agent directed against extracellular human diazepam binding inhibitor (anti-DBI agent) in an amount sufficient to inhibit extracellular human DBI.

In some embodiments, the method of treating cancer comprises administering to the subject (a) an agent directed against extracellular human diazepam binding inhibitor (anti-DBI agent) in an amount sufficient to inhibit extracellular human DBI; and (b) one or more anti-cancer agents. In some embodiments, the method of treating cancer comprises administering to the subject (a) an agent directed against extracellular human diazepam binding inhibitor (anti-DBI agent) in an amount sufficient to inhibit extracellular human DBI; and (b) one or more anti-cancer agents. In some embodiments, the method of treating cancer comprises administering to the subject (a) an agent directed against extracellular human diazepam binding inhibitor (anti-DBI agent) in an amount sufficient to inhibit extracellular human DBI; and (b) one or more corticotherapy agents. In some embodiments, the method of treating cancer comprises administering to the subject (a) an agent directed against extracellular human diazepam binding inhibitor (anti-DBI agent) in an amount sufficient to inhibit extracellular human DBI; (b) one or more anti-cancer agents; and (c) one or more corticotherapy agents, or any combinations thereof. In some embodiments, the administration of the anti-DBI agent, the one or more anti-cancer agents, and/or the one or more corticotherapy agent is in a therapeutic amount sufficient to treat cancer.

In some embodiments, the treatment of cancer is measurable by increased CD8⁺ T cell expression; decreased CD4⁺ T cell expression; decreased T_{reg} cells (CD4⁺Foxp3⁺); an increased ratio of CD8⁺ T cells to T_{reg} cells (CD4⁺Foxp3⁺); increased T_{H}+ cells (CD4⁺Foxp3⁻); decreased expression of Lag3⁺ in T_{H}+ cells (CD4⁺Foxp3⁻); decreased expression of Lag3⁺ in CD8⁺ T cells; decreased expression of Foxp3⁺ in CD4⁺ T cells; decreased T_{reg} ICOS⁺ GITR⁺ Lag3⁻ CD4⁺ T cells; decreased cancer amount; decreased proliferation of cancer cells; increased cancer cell death; decreased cancer growth; increased incidence of survival; incidence of cancer free occurrence; increased time to fatality; or combinations thereof; in a sample relative to a comparable method in the absence of the administration of the anti-DBI agent.

In some embodiments, disclosed herein is a method of treating cancer in a subject in need thereof, the method comprising administering to the subject; (a) an agent directed against extracellular human diazepam binding inhibitor (anti-DBI agent) in an amount sufficient to inhibit extracellular DBI; (b) one or more anti-cancer agents, optionally wherein the one or more anti-cancer agents comprise a chemotherapy agent, an immunotherapy agent, or a combination thereof; (c) one or more corticotherapy agents; or any combinations of (a)-(c), wherein administration of the anti-DBI agent and the one or more anti-cancer agents is sufficient to treat cancer relative to a comparable method in the absence of the anti-DBI agent. In some embodiments, the method of treating cancer comprises administering to the subject two or more of (a) to (c). In some embodiments, the method of treating cancer comprises administering to the subject all of (a) to (c).

In some embodiments, the methods described herein induce an increase in CD8⁺ T cell expression in a sample or a subject as compared to CD8+ T cell expression in the absence of the administration of the anti-DBI agent described herein. In some embodiments, CD8⁺ T cell expression is increased by up to about 10%. In some embodiments, CD8⁺ T cell expression is increased by about 9%. In some embodiments, CD8⁺ T cell expression is increased by about 8%. In some embodiments, CD8⁺ T cells expression is increased by about 7%. In some embodiments, CD8⁺ T cell expression is increased by about 6%. In some embodiments, CD8⁺ T cell expression is increased by about 5%. In some embodiments, CD8⁺ T cell expression is increased by about 4%. In some embodiments, CD8⁺ T cell expression is increased by about 3%. In some embodiments, CD8⁺ T cell expression is increased by about 2%. In some embodiments, CD8⁺ T cell expression is increased by about 1%. In some embodiments, the CD8⁺ T cell expression is increased by about 0.5%. In some embodiments, CD8⁺ T cell expression is increased by about 0.1%.

In some embodiments, the methods described herein induce a decrease in CD4⁺ T cell expression in a sample or a subject as compared to CD4⁺ T cell expression in the absence of the administration of the anti-DBI agent described herein. In some embodiments, CD4⁺ T cell expression is decreased by up to about 5%. In some embodiments, CD4⁺ T cell expression is decreased by about 4%. In some embodiments, CD4⁺ T cell expression is decreased by about 3%. In some embodiments, CD4⁺ T cell expression is decreased by about 2%. In some embodiments, CD4⁺ T cell expression is decreased by about 1%. In some embodiments, CD4⁺ T cell expression is decreased by about 0.5%. In some embodiments, CD4⁺ T cell expression is decreased by about 0.1%.

In some embodiments, the methods described herein induce a decrease in T_{reg} cells (CD4⁺Foxp3⁺) in a sample or a subject relative to a comparable method in the absence of the administration of the anti-DBI agent described herein. In some embodiments T_{reg} cells (CD4⁺Foxp3⁺) expression is decreased by up to about 1%. In some embodiments, T_{reg} cells (CD4⁺Foxp3⁺) expression is decreased by about 0.9%. In some embodiments, T_{reg} cells (CD4⁺Foxp3⁺) expression is decreased by about 0.8%. In some embodiments, T_{reg} cells (CD4⁺Foxp3⁺) expression is decreased by about 0.7%. In some embodiments, T_{reg} cells (CD4⁺Foxp3⁺) expression is decreased by about 0.6%. In some embodiments, T_{reg} cells (CD4⁺Foxp3⁺) expression is decreased by about 0.5%. In some embodiments, T_{reg} cells (CD4⁺Foxp3⁺) expression is decreased by about 0.4%. In some embodiments, T_{reg} cells (CD4⁺Foxp3⁺) expression is decreased by about 0.3%. In some embodiments, T_{reg} cells (CD4⁺Foxp3⁺) expression is decreased by about 0.2%. In some embodiments, T_{reg} cells (CD4⁺Foxp3⁺) expression is decreased by about 0.1%.

In some embodiments, the methods described herein induce an increased ratio of CD8⁺ T cells to T_{reg} cells (CD4⁺Foxp3⁺) in a sample or a subject relative to a comparable method in the absence of the administration of the anti-DBI agent described herein. In some embodiments, the ratio of CD8⁺ T cells to T_{reg} cells (CD4⁺Foxp3⁺) is increased up to about 150:1. In some embodiments, the ratio of CD8⁺ T cells to T_{reg} cells (CD4⁺Foxp3⁺) is increased to about 125.1. In some embodiments, the ratio of CD8⁺ T cells to T_{reg} cells (CD4⁺Foxp3⁺) is increased to about 100:1. In some embodiments, the ratio of CD8⁺ T cells to T_{reg} cells (CD4⁺Foxp3⁺) is increased to about 75:1. In some embodiments, the ratio of CD8⁺ T cells to T_{reg} cells (CD4⁺Foxp3⁺) is increased to about 50:1. In some embodiments, the ratio of CD8⁺ T cells to T_{reg} cells (CD4⁺Foxp3⁺) is increased to about 25:1.

In some embodiments, the methods described herein induce an increase of T_{H}+ cells (CD4⁺Foxp3⁻) in a sample or a subject as compared to the amount of T_{H}+ cells (CD4⁺Foxp3⁻) in a comparative method in the absence of administration of the anti-DBI agent described herein. In some embodiments, T_{H}+ cells (CD4⁺Foxp3⁻) cells are increased up to about 1%. In some embodiments, T_{H}+ cells (CD4⁺Foxp3⁻) cells are increased by about 0.9%. In some embodiments, T_{H}+ cells (CD4⁺Foxp3⁻) cells are increased by about 0.8%. In some embodiments, T_{H}+ cells (CD4⁺Foxp3⁻) cells are increased by about 0.7%. In some embodiments, T_{H}+ cells (CD4⁺Foxp3⁻) cells are increased by about 0.6%. In some embodiments, T_{H}+ cells (CD4⁺Foxp3⁻) cells are increased by about 0.5%. In some embodiments, T_{H}+ cells (CD4⁺Foxp3⁻) cells are increased by about 0.4%. In some embodiments, T_{H}+ cells (CD4⁺Foxp3⁻) cells are increased by about 0.3%. In some embodiments, T_{H}+ cells (CD4⁺Foxp3⁻) cells are increased by about 0.2%. In some embodiments, T_{H}+ cells (CD4⁺Foxp3⁻) cells are increased by about 0.1%.

In some embodiments, the methods described herein induce a decrease in the expression of Lag3⁺ in T_{H}+ cells (CD4⁺Foxp3⁻) in a sample or a subject relative to a comparable method in the absence of the administration of the anti-DBI agent described herein as well as agents, compositions and combinations for use in such methods. In some embodiments, the expression of Lag3+ in T_{H}+ cells (CD4⁺Foxp3⁻) is decreased by up to about 10%. In some embodiments, the expression of Lag3⁺ in T_{H}+ cells (CD4⁺Foxp3⁻) is decreased by about 9%. In some embodiments, the expression of Lag3⁺ in T_{H}+ cells (CD4⁺Foxp3⁻) is decreased by about 8%. In some embodiments, the expression of Lag3⁺ in T_{H}+ cells (CD4⁺Foxp3⁻) is decreased by about 7%. In some embodiments, the expression of Lag3⁺ in T_{H}+ cells (CD4⁺Foxp3⁻) is decreased by about 6%. In some embodiments, the expression of Lag3⁺ in T_{H}+ cells (CD4⁺Foxp3⁻) is decreased by about 5%. In some embodiments, the expression of Lag3⁺ in T_{H}+ cells (CD4⁺Foxp3⁻) is decreased by about 4%. In some embodiments, the expression of Lag3⁺ in T_{H}+ cells (CD4⁺Foxp3⁻) is decreased by about 3%. In some embodiments, the expression of Lag3⁺ in T_{H}+ cells (CD4⁺Foxp3⁻) is decreased by about 2%. In some embodiments, the expression of Lag3+ in T_{H}+ cells (CD4⁺Foxp3⁻) is decreased by about 1%.

In some embodiments, the methods described herein induce a decrease in the expression of Lag3⁺ in CD8⁺ T cells in a sample or a subject relative to a comparable method in the absence of the administration of the anti-DBI agent described herein as well as agents, compositions and combinations for use in such methods. In some embodiments, the expression of Lag3⁺ in CD8⁺ T cells is decreased by up to about 10%. In some embodiments, the expression of Lag3⁺ in CD8⁺ T cells is decreased by about 9%. In some embodiments, the expression of Lag3⁺ in CD8⁺ T cells is decreased by about 8%. In some embodiments, the expression of Lag3⁺ in CD8⁺ T cells is decreased by about 7%. In some embodiments, the expression of Lag3⁺ in CD8⁺ T cells is decreased by about 6%. In some embodiments, the expression of Lag3⁺ in CD8⁺ T cells is decreased by about 5%. In some embodiments, the expression of Lag3⁺ in CD8⁺ T cells is decreased by about 4%. In some embodiments, the expression of Lag3⁺ in CD8⁺ T cells is decreased by about 3%. In some embodiments, the expression of Lag3⁺ in CD8⁺ T cells is decreased by about 2%. In some embodiments, the expression of Lag3⁺ in CD8⁺ T cells is decreased by about 1%.

In some embodiments, the methods described herein induce a decrease in the expression of Foxp3⁺ in CD4⁺ T cells in a sample or a subject relative to a comparable method in the absence of the anti-DBI agent described herein. In some embodiments, the expression of Foxp3⁺ in CD4⁺ T cells is decreased by up to about 20%. In some embodiments, the expression of Foxp3⁺ in CD4⁺ T cells is decreased by about 19%. In some embodiments, the expression of Foxp3⁺ in CD4⁺ T cells is decreased by about 18%. In some embodiments, the expression of Foxp3⁺ in CD4⁺ T cells is decreased by about 17%. In some embodiments, the expression of Foxp3⁺ in CD4⁺ T cells is decreased by about 16%. In some embodiments, the expression of Foxp3⁺ in CD4⁺ T cells is decreased by about 15%. In some embodiments, the expression of Foxp3⁺ in CD4⁺ T cells is decreased by about 14%. In some embodiments, the expression of Foxp3⁺ in CD4⁺ T cells is decreased by about 13%. In some embodiments, the expression of Foxp3⁺ in CD4⁺ T cells is decreased by about 12%. In some embodiments, the expression of Foxp3⁺ in CD4⁺ T cells is decreased by about 11%. In some embodiments, the expression of Foxp3 in CD4⁺ T cells is decreased by about 10%. In some embodiments, the expression of Foxp3⁺ in CD4⁺ T cells is decreased by about 9%. In some embodiments, the expression of Foxp3⁺ in CD4⁺ T cells is decreased by about 8%. In some embodiments, the expression of Foxp3⁺ in CD4⁺ T cells is decreased by about 7%. In some embodiments, the expression of Foxp3⁺ in CD4⁺ T cells is decreased by about 6%. In some embodiments, the expression of Foxp3⁺ in CD4⁺ T cells is decreased by about 5%. In some embodiments, the expression of Foxp3⁺ in CD4⁺ T cells is decreased by about 4%. In some embodiments, the expression of Foxp3⁺ in CD4⁺ T cells is decreased by about 3%. In some embodiments, the expression of Foxp3⁺ in CD4⁺ T cells is decreased by about 2%. In some embodiments, the expression of Foxp3⁺ in CD4⁺ T cells is decreased by about 1%.

In some embodiments, the methods described herein induce a decrease in T_{reg} ICOS⁺ GITR⁺ Lag3⁻ CD4⁺ T cells in a sample or a subject relative to a comparable method in the absence of the administration of the anti-DBI agent described herein. In some embodiments, ICOS⁺ GITR⁺ Lag3⁻ CD4⁺ T cells are decreased by up to about 30%. In some embodiments, ICOS⁺ GITR⁺ Lag3⁻ CD4⁺ T cells are decreased by about 29%. In some embodiments, ICOS⁺ GITR⁺ Lag3⁻ CD4⁺ T cells are decreased by about 28%. In some embodiments, ICOS⁺ GITR⁺ Lag3⁻ CD4⁺ T cells are decreased by about 27%. In some embodiments, ICOS⁺ GITR⁺ Lag3⁻ CD4⁺ T cells are decreased by about 26%. In some embodiments, ICOS⁺ GITR⁺ Lag3⁻ CD4⁺ T cells are decreased by about 25%. In some embodiments, ICOS⁺ GITR⁺ Lag3⁻ CD4⁺ T cells are decreased by about 24%. In some embodiments, ICOS⁺ GITR⁺ Lag3⁻ CD4⁺ T cells are decreased by about 23%. In some embodiments, ICOS⁺ GITR⁺ Lag3⁻ CD4⁺ T cells are decreased by about 22%. In some embodiments, ICOS⁺ GITR⁺ Lag3⁻ CD4⁺ T cells are decreased by about 21%. In some embodiments, ICOS⁺ GITR⁺ Lag3⁻ CD4⁺ T cells are decreased by about 20%. In some embodiments, ICOS⁺ GITR⁺ Lag3⁻ CD4⁺ T cells are decreased by about 19%. In some embodiments, ICOS⁺ GITR⁺ Lag3⁻ CD4⁺ T cells are decreased by about 18%. In some embodiments, ICOS⁺ GITR⁺ Lag3⁻ CD4⁺ T cells are decreased by about 17%. In some embodiments, ICOS⁺ GITR⁺ Lag3⁻ CD4⁺ T cells are decreased by about 16%. In some embodiments, ICOS⁺ GITR⁺ Lag3⁻ CD4⁺ T cells are decreased by about 15%. In some embodiments, ICOS⁺ GITR⁺ Lag3⁻ CD4⁺ T cells are decreased by about 14%. In some embodiments, ICOS⁺ GITR⁺ Lag3⁻ CD4⁺ T cells are decreased by about 13%. In some embodiments, ICOS⁺ GITR⁺ Lag3⁻ CD4⁺ T cells are decreased by about 12%. In some embodiments, ICOS⁺ GITR⁺ Lag3⁻ CD4⁺ T cells are decreased by about 11%. In some embodiments, ICOS⁺ GITR⁺ Lag3⁻ CD4⁺ T cells are decreased by about 10%. In some embodiments, ICOS⁺ GITR⁺ Lag3⁻ CD4⁺ T cells are decreased by about 9%. In some embodiments, ICOS⁺ GITR⁺ Lag3⁻ CD4⁺ T cells are decreased by about 8%. In some embodiments, ICOS⁺ GITR⁺ Lag3⁻ CD4⁺ T cells are decreased by about 7%. In some embodiments, ICOS⁺ GITR⁺ Lag3⁻ CD4⁺ T cells are decreased by about 6%. In some embodiments, ICOS⁺ GITR⁺ Lag3⁻ CD4⁺ T cells are decreased by about 5%. In some embodiments, ICOS⁺ GITR⁺ Lag3⁻ CD4⁺ T cells are decreased by about 4%. In some embodiments, ICOS⁺ GITR⁺ Lag3⁻ CD4⁺ T cells are decreased by about 3%. In some embodiments, ICOS⁺ GITR⁺ Lag3⁻ CD4⁺ T cells are decreased by about 2%. In some embodiments, ICOS⁺ GITR⁺ Lag3⁻ CD4⁺ T cells are decreased by about 1%.

In some embodiments, the methods described herein maintain the ratio of CD8⁺ T cells to T_{H}+ cells (CD4⁺Foxp3⁻) in a sample or a subject relative to a comparable method in the absence of the anti-DBI agent described herein. In some embodiments, the ratio of CD8⁺ T cells to T_{H}+ cells (CD4⁺Foxp3⁻) is maintained within about 3%. In some embodiments, the ratio of CD8⁺ T cells to T_{H}+ cells (CD4⁺Foxp3⁻) is maintained within about 2%. In some embodiments, the ratio of CD8⁺ T cells to T_{H}+ cells (CD4⁺Foxp3⁻) is maintained within about 1%. In some embodiments, the ratio of CD8⁺ T cells to T_{H}+ cells (CD4⁺Foxp3⁻) is maintained within about 0.5%. In some embodiments, the ratio of CD8⁺ T cells to T_{H}+ cells (CD4⁺Foxp3⁻) is maintained within about 0.1%.

In some embodiments, the methods described herein maintain the ratio of CD8⁺ T cells to CD4⁺ T cells in a sample or a subject relative to a comparable method in the absence of the administration of the anti-DBI agent described herein. In some embodiments, the ratio of CD8⁺ T cells to CD4⁺ T cells is maintained within about 1%. In some embodiments, the ratio of CD8⁺ T cells to CD4⁺ T cells is maintained within about 0.9%. In some embodiments, the ratio of CD8⁺ T cells to CD4⁺ T cells is maintained within about 0.8%. In some embodiments, the ratio of CD8⁺ T cells to CD4⁺ T cells is maintained within about 0.7%. In some embodiments, the ratio of CD8⁺ T cells to CD4⁺ T cells is maintained within about 0.6%. In some embodiments, the ratio of CD8⁺ T cells to CD4⁺ T cells is maintained within about 0.5%. In some embodiments, the ratio of CD8⁺ T cells to CD4⁺ T cells is maintained within about 0.4%. In some embodiments, the ratio of CD8⁺ T cells to CD4⁺ T cells is maintained within about 0.3%. In some embodiments, the ratio of CD8⁺ T cells to CD4⁺ T cells is maintained within about 0.2%. In some embodiments, the ratio of CD8⁺ T cells to CD4⁺ T cells is maintained within about 0.1%.

In some embodiments, the methods described herein maintain the incidence of T_{H} ICOS^{int} GITR⁺ Lag3⁻ PD1⁻ CD4⁺ T cells in a sample or a subject relative to a comparable method in the absence of the administration of the anti-DBI agent. In some embodiments, the incidence of T_{H} ICOS^{int} GITR⁺ Lag3⁻ PD1⁻ CD4⁺ T cells is maintained within about 1%. In some embodiments, the incidence of T_{H} ICOS^{int} GITR⁺ Lag3⁻ PD1⁻ CD4⁺ T cells is maintained within about 0.9%. In some embodiments, the incidence of T_{H} ICOS^{int} GITR⁺ Lag3⁻ PD1⁻ CD4⁺ T cells is maintained within about 0.8%. In some embodiments, the incidence of T_{H} ICOS^{int} GITR⁺ Lag3⁻ PD1⁻ CD4⁺ T cells is maintained within about 0.7%. In some embodiments, the incidence of T_{H} ICOS^{int} GITR⁺ Lag3⁻ PD1⁻ CD4⁺ T cells is maintained within about 0.6%. In some embodiments, the incidence of T_{H} ICOS^{int} GITR⁺ Lag3⁻ PD1⁻ CD4⁺ T cells is maintained within about 0.5%. In some embodiments, the incidence of T_{H} ICOS^{int} GITR⁺ Lag3⁻ PD1⁻ CD4⁺ T cells is maintained within about 0.4%. In some embodiments, the incidence of T_{H} ICOS^{int} GITR⁺ Lag3⁻ PD1⁻ CD4⁺ T cells is maintained within about 0.3%. In some embodiments, the incidence of T_{H} ICOS^{int} GITR⁺ Lag3⁻ PD1⁻ CD4⁺ T cells is maintained within about 0.2%. In some embodiments, the incidence of T_{H} ICOS^{int} GITR⁺ Lag3⁻ PD1⁻ CD4⁺ T cells is maintained within about 0.1%.

In some embodiments, cancer volume is decreased up to about 60%. In some embodiments, cancer volume is decreased up to about 50%. In some embodiments, cancer volume is decreased by about 40%. In some embodiments, cancer volume is decreased by about 30%. In some embodiments, cancer volume is decreased by about 20%. In some embodiments, cancer volume is decreased by about 10%. In some embodiments, cancer volume is decreased by about 5%.

In some embodiments, the decreased cancer amount induces a decrease in cancer area relative to a comparable method in the absence of the administration of the anti-DBI agent described herein.

In some embodiments, the methods described herein induce a decrease in cancer cell proliferation in a sample or a subject relative to a comparable method in the absence of the administration of the anti-DBI agent described herein at a comparable time point.

In some embodiments, the methods described herein induce an increase in cancer cell death in a sample or a subject relative to a comparable method in the absence of the administration of the anti-DBI agent described herein.

In some embodiments, the methods described herein induce a decrease in cancer growth in a subject or a sample relative to a comparable method in the absence of the administration of the anti-DBI agent described herein.

### Therapeutic Regimens

The present disclosure also provides for a therapeutic regimen for use as an anti-cancer therapy as well as agents, compositions and combinations for use in such regimens. In some embodiments, the therapeutic regimen comprises (a) an agent directed against extracellular human diazepam binding inhibitor (anti-DBI agent) in an amount sufficient to inhibit extracellular human DBI; and (b) one or more anti-cancer agents. In some embodiments, the anti-DBI agent and the one or more anti-cancer agents are present in the therapeutic regimen in an amount sufficient to induce one or more immunosurveillance biomarkers in a subject upon administration to the subject. In some embodiments, the therapeutic regimen causes an enhanced therapeutic effect of the anti-cancer therapy relative to a comparable regimen absent the administration of the anti-DBI agent. In some embodiments, the absence of the administration of the anti-DBI agent is the administration of the anti-cancer agent alone or the administration the corticotherapy agent alone. In some embodiments, the absence of the administration of the anti-DBI agent is the administration of anti-cancer agent together with the corticotherapy.

The present disclosure further provides for a therapeutic regimen for treating cancer as well as agents, compositions and combinations for use in such regimens. In some embodiments, the therapeutic regimen comprises (a) an agent directed against extracellular human diazepam binding inhibitor (anti-DBI agent) in an amount sufficient to inhibit extracellular human DBI; and (b) one or more anti-cancer agents. In some embodiments, the anti-DBI agent and the one or more anti-cancer agents are present in the therapeutic regimen in an amount sufficient to inhibit one or more cancer progression biomarkers in a subject upon administration to the subject relative to a comparable regimen absent the administration of the anti-DBI-agent. In some embodiments, the absence of the administration of the anti-DBI agent is the administration of the anti-cancer agent alone or the administration the corticotherapy agent alone. In some embodiments, the absence of the administration of the anti-DBI agent is the administration of the anti-cancer agent together with the corticotherapy agent.

In some embodiments, the anti-DBI agent and the one or more anti-cancer agents are separate components that are administered together. In some embodiments, the anti-DBI agent and the one or more anti-cancer agents are separate components that are administered sequentially.

In some embodiments, the anti-DBI agent is administered at least about four weeks prior to the one or more anti-cancer agents. In some embodiments, the anti-DBI agent is administered at least about three weeks prior to the one or more anti-cancer agents. In some embodiments, the anti-DBI agent is administered at least about two weeks prior to the one or more anti-cancer agents. In some embodiments, the anti-DBI agent is administered at least about one week prior to the one or more anti-cancer agents.

In some embodiments, the anti-DBI agent and the one or more anti-cancer agents are co-administered.

In some embodiments, the one or more corticotherapy agents are co-administered with the anti-DBI agent and the one or more anti-cancer agents. In some embodiments, the one or more corticotherapy agents are co-administered with the anti-DBI agent. In some embodiments, the one or more corticotherapy agent are co-administered with the one or more anti-cancer agent. In some embodiments, the one or more corticotherapy agents are co-administered with the anti-DBI agent and the one or more anti-cancer agents. In some embodiments, the one or more corticotherapy agents are co-administered with the anti-DBI agent. In some embodiments, the one or more corticotherapy agents are co-administered with the one or more anti-cancer agents.

In some embodiments, the one or more corticotherapy agents are administered sequentially with the anti-DBI agent and the one or more anti-cancer agents. In some embodiments, the one or more corticotherapy agents are administered sequentially with the anti-DBI agent. In some embodiments, the one or more corticotherapy agents are administered sequentially with the one or more anti-cancer agents. In some embodiments, the one or more corticotherapy agents are administered sequentially with the anti-DBI agent and the one or more anti-cancer agents. In some embodiments, the one or more corticotherapy agents are administered sequentially with the anti-DBI agent. In some embodiments, the one or more corticotherapy agents are administered sequentially with the one or more anti-cancer agents.

In some embodiments, any two or more of the anti-DBI agents, the one or more anti-cancer agents and the one or more corticotherapy agents are co-administered.

### Cancers

In some embodiments, the cancer is characterized by an inhibitory tumor microenvironment. In some embodiments, cancers described herein make use of effective immune recognition and other mechanisms to avoid elimination by the immune system. In some embodiments, cancers, such as immunocompetent tumors, produce an inhibitory microenvironment to down-regulate the immune response. In some embodiment, an inhibitory cancer microenvironment includes the expression of immunosuppressive cells and factors (such as regulatory T cells, cancer-associated fibroblasts, myeloid derived inhibitory cells, and other cell types), hypoxia, low pH, and exhibit other intrinsic and dynamic immunosuppressive characteristics. For example, a cancer microenvironment can be intrinsically immunosuppressive to protect cancer cells from immune surveillance, but also dynamically adaptive to accommodate rapid tumor growth and progression and to counter any stress and insult conditions, such as chemotherapy.

In some embodiments of the present disclosure, the cancer is a refractory cancer. In some embodiments, the cancer does not respond to treatment. In some embodiments, the cancer cells have ways of defending themselves against chemotherapy drugs. In some embodiments, the cancer cells have ways of defending themselves against immunotherapy drugs. In some embodiments, the cancer cells have ways of defending themselves against biological agents. In some embodiments, the cancer cells have ways of defending themselves against radiation therapy.

In some embodiments, the cancer is resistant to immune checkpoint inhibitor therapy. In some embodiments, the cancer is selected from solid tumors, blood-borne tumors, cancers of skin, cancers of tissues, cancers of organs, cancers of bone, cancers of cartilage, cancers of blood, cancers of vessels, primary cancers, metastatic cancers, bladder cancer, bone marrow cancer, brain cancer, breast cancer, colon cancer, esophagus cancer, gastro-intestine cancer, gum cancer, kidney cancer, liver cancer, lung cancer, nasopharynx cancer, neck cancer, ovary cancer, prostate cancer, stomach cancer, testis cancer, tongue cancer, and uterus cancer. In some embodiments, the cancer is a solid tumor cancer. In some embodiments, the cancer is a fibrous cancer.

In some embodiments, the cancer is selected from carcinomas, lung cancer, non-small cell lung cancer, breast cancer, neoplasm carcinoma, undifferentiated carcinoma, giant cell carcinoma, spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary adenocarcinoma, follicular adenocarcinoma, non-encapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, Paget's disease, mammary carcinoma, acinar cell carcinoma, adenosquamous carcinoma, adenocarcinoma with squamous metaplasia, thymoma, ovarian stromal tumor, thecoma, granulosa cell tumor, roblastoma, Sertoli cell carcinoma, leydig cell tumor, lipid cell tumor, paraganglioma, extra-mammary paraganglioma, pheochromocytoma, glomangiosarcoma, melanoma, amelanotic melanoma, superficial spreading melanoma, melanoma in giant pigmented nevus, epithelioid cell melanoma, blue nevus, sarcoma, fibrosarcoma, fibrous histiocytoma, myxosarcoma, liposarcoma, leiomyosarcoma, rhabdomyosarcoma, embryonal rhabdomyosarcoma, alveolar rhabdomyosarcoma, stromal sarcoma, mixed tumor, mullerian mixed tumor, nephroblastoma, hepatoblastoma, carcinosarcoma, mesenchymoma, brenner tumor, phyllodes tumor, synovial sarcoma, mesothelioma, dysgerminoma, embryonal carcinoma, teratoma, struma ovarii, choriocarcinoma, mesonephroma, hemangiosarcoma; hemangioendothelioma, kaposi's sarcoma, hemangiopericytoma, lymphangiosarcoma, osteosarcoma, juxtacortical osteosarcoma, chondrosarcoma, chondroblastoma, mesenchymal chondrosarcoma, giant cell tumor of bone, ewing's sarcoma, odontogenic tumor, ameloblastic odontosarcoma, ameloblastoma, ameloblastic fibrosarcoma, pinealoma, chordoma, glioma, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, neurofibrosarcoma, neurilemmoma, granular cell tumor, lymphoma, Hodgkin's disease, Hodgkin's lymphoma, paragranuloma, lymphoma, small lymphocytic, large cell diffuse lymphoma, follicular lymphoma, mycosis fungoides, other specified non-Hodgkin's lymphomas, histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, and hairy cell leukemia. In some embodiments, the cancer is a malignant cancer.

In some embodiments, the cancer is selected from carcinomas, lung cancer, non-small cell lung cancer, and breast cancer. In some embodiments, the cancer is a carcinoma. In some embodiments, the cancer is lung cancer. In some embodiments, the cancer is non-small cell lung cancer. In some embodiments, the cancer is breast cancer.

In some embodiments, the cancer is a solid tumor cancer. In some embodiments, the cancer is a fibrous tumor. In some embodiments, the subject is human.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1; Role of DBI in Immunotherapy and Corticotherapy

### Materials and Methods

### Cell Culture and Treatment

Syngeneic cancer cell lines were maintained in Dulbecco's Modified Eagle Medium (fibrosarcoma cell line MCA205, lung carcinoma TC-1) or Roswell Park Memorial Institute medium 1640 (breast adenocarcinoma E0771), both supplemented with HEPES and 10% fetal bovine serum, and sub-cultured thrice weekly.

### Mouse Experiments

C57BL/6J mice were housed under temperature-controlled conditions and provided with food and water ad libidum. Experimentations were conducted under the FELASA guidelines and approved by the local Ethics committee (project numbers #24410; #31018; 2020_021_24771, 2021_009_29509, 2021_010_29520).

### Subcutaneous Tumor Models

MCA205—Mice were anesthetized lightly with isoflurane and injected subcutaneously with 0.3×10⁶ MCA205 cells. Therapy consisted of oxaliplatin (Sigma, 10 mg/kg), administered when medium tumor size reached 50 mm³, followed by 3 doses of neutralizing anti-PD1 monoclonal antibody (BioXCell, clone 29F.1.A12, 200 μg per mouse 8, 12 and 16 days after chemotherapy). This therapy or its corresponding control (Vehicle+anti-PD1 isotype IgG2A) was coupled when indicated with six injections of neutralizing anti-DBI monoclonal antibody (2.5 mg/kg) or its isotype (IgG2A, 2.5 mg/kg) from two days before chemotherapy until one day before immunotherapy. Tumor size was monitored thrice weekly until one of the following endpoints were reached; tumor size>1500 mm³, ulceration or weight loss>20%.

E0771—For the establishment of syngeneic solid tumors, C57BL/6J mice (8-weeks-old female) were injected subcutaneously (s.c.) with 5×10⁵ wild type E0771 in the right lower quadrant of the abdomen alongside of the mammary gland (close to the orthotopic site). Approximately 7 days after injection, tumor growth was monitored with an electronic caliper and mice were assigned to different groups. From day 8, mice were treated with a neutralizing anti-DBI monoclonal antibody (5 mg/kg) or its isotype (IgG2A, 5 mg/kg) every two days. At the same time, free corticosterone (Sigma-Aldrich) was added to the drinking water (0.1 mg/ml; water/ethanol 0.66%). Anti-PD1 monoclonal antibody (αPD1; 10 mg/kg) was injected at days 12, 15, 18 and 21. Tumor growth was monitored routinely with an electronic caliper.

TC-1—To establish the orthotopic TC1 model, wild type TC1 Luc cells (5×10⁵ in 100 μL PBS) were intravenously injected to wild type C57BL/6J mice. Tumor incidence and development were monitored by in vivo photonic imaging of tumor cells' luciferase activity. About 7 days after injection, tumor incidence in the lung was detected at an exposure time of 4 min, and mice were randomized according to quantified tumor sizes and assigned to different groups for treatment as described below. Anti-DBI or isotype control antibody was intraperitoneally (i.p.) injected on day 9, at the dose of 2.5 mg/kg body weight, and on day 10, then maintained every other day until day 18. Oxaliplatin or its vehicle (PBS) was injected i.p. on day 11 at the dose of 5 mg/kg body weight; PD1 monoclonal antibody or its isotype was injected at the dose of i.p. on day 19, day 23 and day 27. Anti-DBI or its isotype control was injected one day before each anti-PD1 and continuously maintained at a pace of once a week.

For the acquisition of bioluminescence images, mice received an i.p. injection of luciferase substrate (Beetle Luciferin potassium salt, Promega) at a dose of 3 mg per mouse, and 8 min (for TC1 model) post luciferin inoculation, photons were acquired on an IVIS LUMINA III bioluminescence in vivo imaging system (PerkinElmer, Waltham, MA, USA). In vivo imaging was conducted every 4-5 days with an exposure time starting with 4 min, which then was gradually decreased to 3 min, 2 min, and 1 min when photon saturation occurred. Tumor bearing mice showing photon saturation at 1 min of exposure were euthanized.

### Anti-Tumor Vaccination Assays

Two weeks before vaccination (day-14), C57BL/6J mice (8-weeks-old female) were pretreated with a neutralizing anti-DBI monoclonal antibody (5 mg/kg) or its isotype (IgG2A, 5 mg/kg) every two days (i.p., in 200 μl PBS) and free corticosterone (Sigma-Aldrich) was added to the drinking water (0.1 mg/ml; ethanol/water 0.66%). Wild-type MCA205 cells were treated with MTX (4 μM) for 24 h. Then supernatants and detached cells were collected and centrifuged, and the pellet was further washed with ice-cold PBS. 2×10⁶ cells in 100 μl PBS were s.c. injected to the left flank of immunocompetent C57BL/6J mice (day −7). PBS was injected as a negative control. One week later, all mice were confirmed tumor-free in the vaccination flank and living cancer cells (2×10⁵ cells per mouse for MCA205 cells) were injected in the right flank of vaccinated mice (day 0). Corticosterone and neutralizing anti-DBI monoclonal antibody were continually administrated throughout the experiment. Tumor growth was regularly monitored for the following weeks.

### Induction of Autoimmunity Against ACBP DBI

Anti-ACBP/DBI autoimmunity was induced in young adult mice (6-8 weeks) by four weekly doses of KLH-ACBP/DBI vaccine or its non ACBP/DBI-specific control KLH (77649, Thermo Fisher Scientific), injected together with the mineral adjuvant Montanide ISA 51 VG (Seppic) as previously described [Montégut L. et al., Immunization of mice with the self-peptide ACBP coupled to keyhole limpet hemocyanin, STAR Protoc, 2022, 3 (1), p. 101095.] Auto-immunity was confirmed by immunoblotting of RecDBI revealed by mouse plasma. After two weeks of immune recovery, mice were injected subcutaneously with 0.3×10⁶ MCA205 cells in the right flank and treatment was continued as described above.

### Immune Infiltrate in the MCA205 Tumor Microenvironment

Ten days after chemotherapy, MCA205 tumors from three independent experiments were dissected and homogenized to single-cell suspensions by mechanical and enzymatic disruption, following the manufacturer's instruction (Tumor dissociation kit, 130-096-730, Miltenyi Biotec). Cells were stained with a viability staining (Live-Dead Fixable Yellow Dye, Thermo Fisher Scientific), then Fc receptors were blocked by an uncoupled anti-mouse CD16/CD32 antibody (BD BioSciences; clone 2.4G2) and fluorophore-coupled antibodies were added for the detection of surface markers (CD45, CD3, CD4, CD8a, ICOS, GITR, LAG3, PD1, TIGIT and VISTA). Cells were fixed and permeabilized (eBioscience FoxP3/Transcription Factor staining buffer, Thermo Fisher Scientific) prior to staining with anti-FoxP3. Fluorescence data was acquired on BD LSRFortessa X20 with the BD FACS Diva software. Compensation, scaling, gating and data analysis were performed on the omiq.ai platform. Specific subpopulations of interest were defined among CD4⁺ and CD8⁺ T cells by performing unsupervised clustering (FlowSOM algorithm) on one experiment set. Gating strategies were inferred from the markers expressed by the clusters of interest differentially present between the OXA+anti-PD1 and anti-DBI+OXA+anti-PD1 groups (FoxP3⁻, ICOS^{int}, GITR⁺, Lag3⁻ activated T_{H}, FoxP3⁺, GITR⁺, Lag3⁻ activated T_{reg}) and applied to the three independent replicates for statistical analysis.

### Statistical Analysis.

For tumor growth experiments, longitudinal analyses were performed by linear mixed effect modeling of tumor sizes. Type II ANOVA (Wald tests) was used to compute p-values by testing jointly that both tumor growth slopes and intercepts were the same between treatment groups of interest, and p-values were corrected for multiple comparisons by the Holm method. Survival curves were compared two-by-two by Log-rank Mantel-Cox test in the GraphPad Prism (v9) software. For comparison of immune cells infiltration, data from the population relative counts was cleaned up by ROUT test (outlier threshold=1%) and statistical significance was tested by one-way ANOVA with Sidak correction for multiple comparisons.

### Results

### Improvement of Chemo-Immunotherapy Outcome by Autoantibodies Neutralizing ACBP DBI

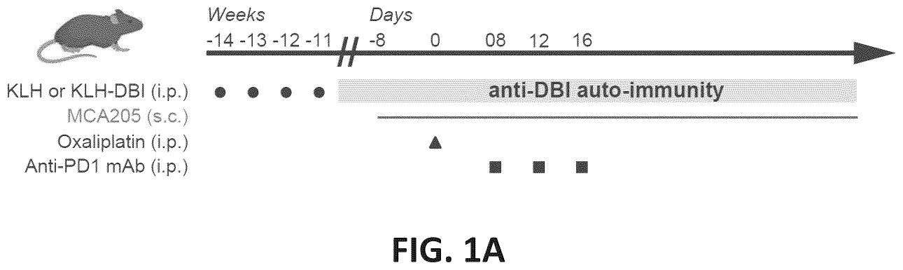

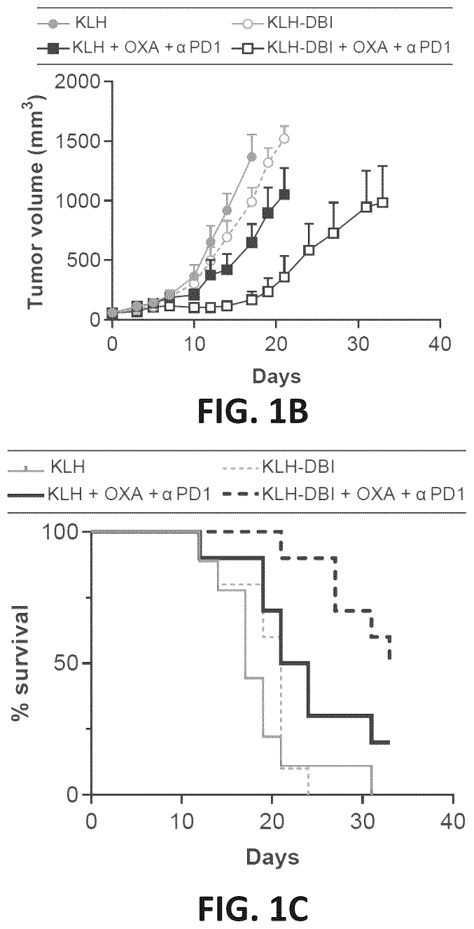

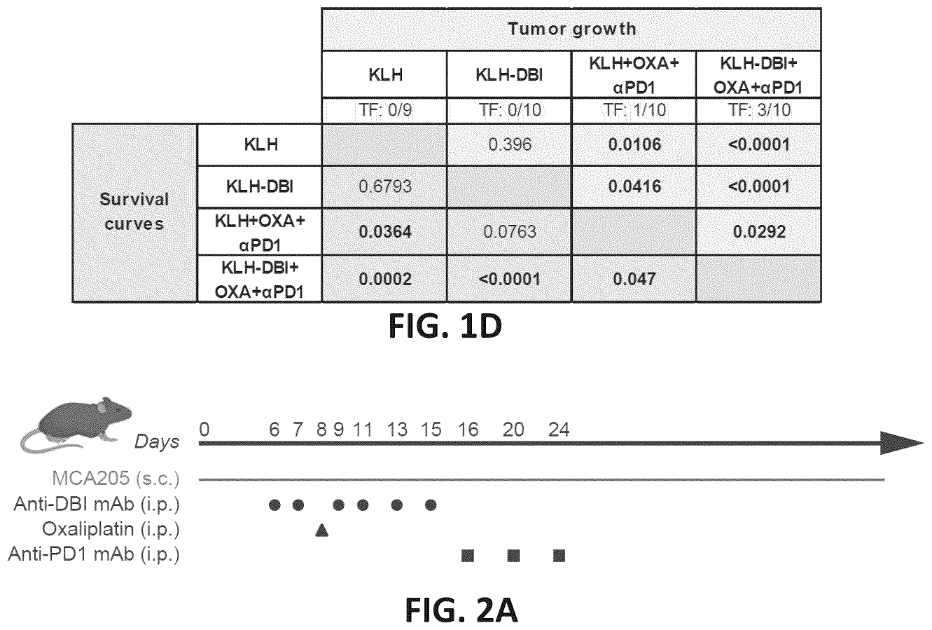

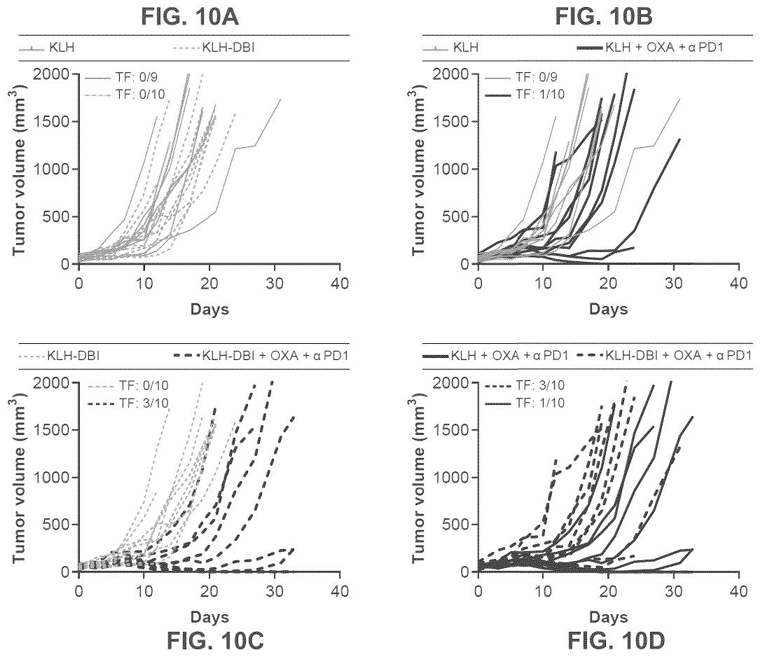

As a first approach to investigate the impact of ACBP/DBI on chemo-immunotherapy,
6-week-old C57BL/6J mice were immunized with the immunogenic carrier protein keyhole limpet hemocyanin (KLH) alone (as a control) or KLH conjugated to ACBP/DBI with the adjuvant montanide. Four repeated injections of KLH-ACBP/DBI conjugate during consecutive weeks are known to induce a strong auto-antibody response against ACBP/DBI, resulting in its neutralization. [Montégut, L. et al., Immunization of mice with the self-peptide ACBP coupled to keyhole limpet hemocyanin, STAR Protoc, 2022, 3(1), p. 101095.] Two weeks after the termination of vaccination protocol, MC205 cutaneous fibrosarcoma cells (which are syngeneic with C57BL/6J) were inoculated subcutaneously (s.c). When tumors were palpable (surface of 25 to 50 mm²), around day 8 post-inoculation, the mice received one cycle of intraperitoneal (i.p.) chemotherapy with oxaliplatin (OXA or PBS as a vehicle control) followed by three cycles of immunotherapy consisting of the i.p. injection of a PD-1-specific mAb (anti-PD1 or an isotype-matched control mAb) injected on days 16, 20 and 24 post-inoculation (FIG. 1A). Tumor growth was monitored by means of a caliper (FIG. 1B and FIG. 10A-FIG. 10D), and the survival of mice was recorded (FIG. 1C). Of note, tumors implanted in KLH-ACBP/DBI vaccinated mice responded more efficiently to chemo-immunotherapy with OXA+anti-PD1 than tumors implanted in KLH-vaccinated control animals. This improved therapeutic effect was statistically significant at the levels of tumor growth and animal survival (FIG. 1A-FIG. 1D). Altogether, these results indicate that autoantibodies neutralizing ACBP/DBI can improve the outcome of chemo-immunotherapy.

### Improvement of Chemo-Immunotherapy Outcome by Anti-ACBP/DBI mAb

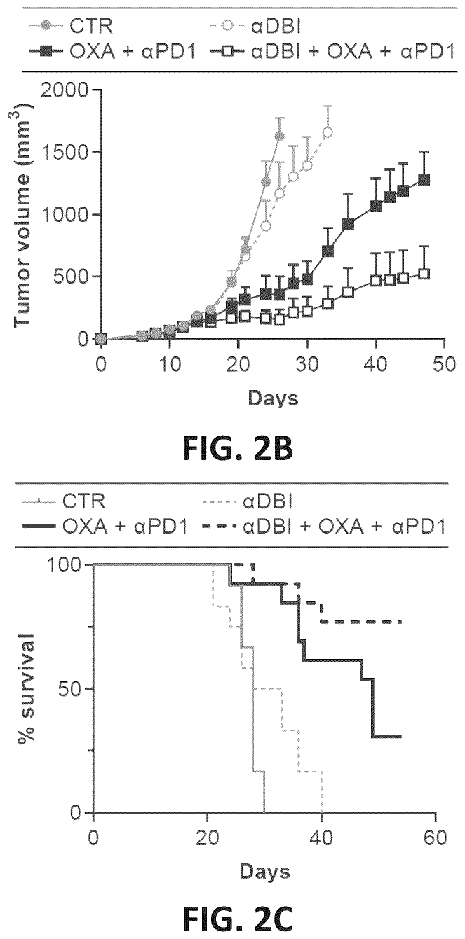

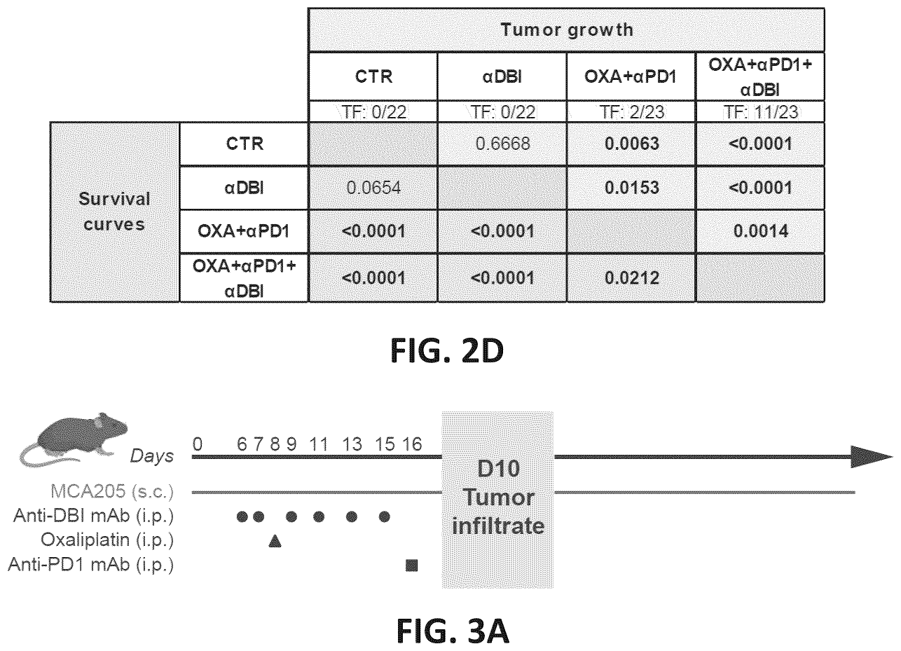

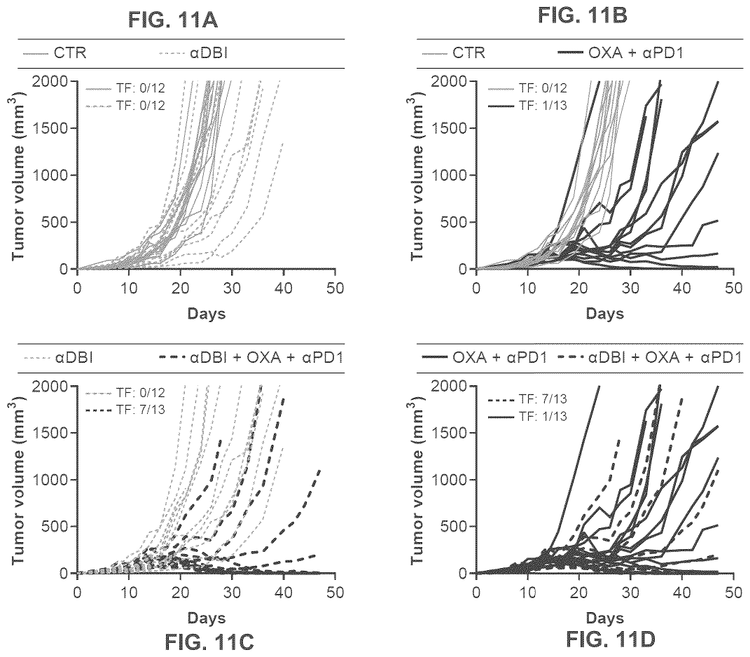

In the next experiment, MC205 cells were s.c. injected into 8-week-old C57BL/6J mice that received periodic injections of anti-ACBP/DBI mAb (or as a control an IgG2A isotype control antibody) from day 6 post-inoculation, i.p. OXA chemotherapy once on day 8 and immunotherapy auto-antibody (i.e., an anti-PD1 mAb) in 3 cycles on days 16, 20 and 24 post-inoculation (FIG. 2A). Tumors implanted in anti-ACBP/DBI mAb-treated mice responded more efficiently to chemo-immunotherapy with OXA+anti-PD1 than tumors implanted in control animals, leading to significantly reduced tumor growth with complete regression of the cancers in 11 out of 23 mice in anti-ACBP/DBI mAb-treated mice receiving chemoimmunotherapy but only 2 out of 23 animals receiving chemo-immunotherapy alone. This translated also in a significant increase in animal survival (FIG. 2B-FIG. 2D and FIG. 11A-FIG. 11D). Hence, administration of a mAb specific of ACBP/DBI improves the efficacy of chemo-immunotherapy.

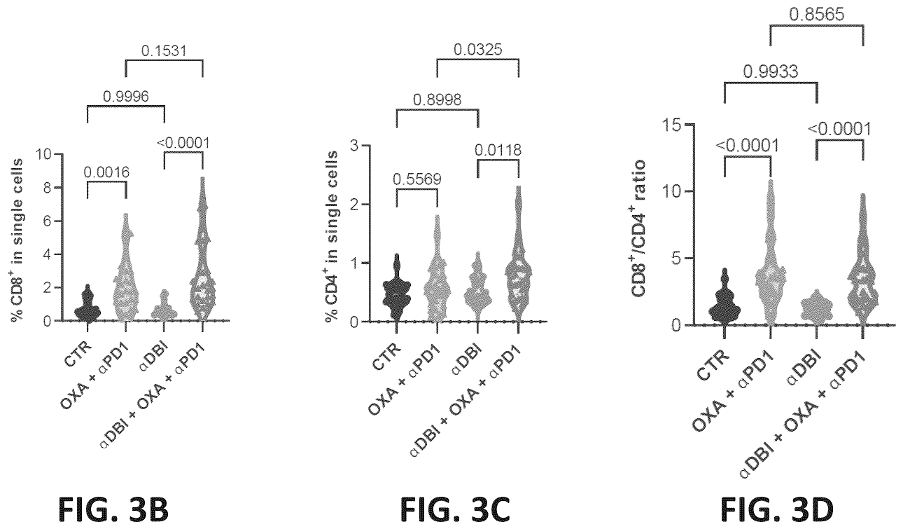

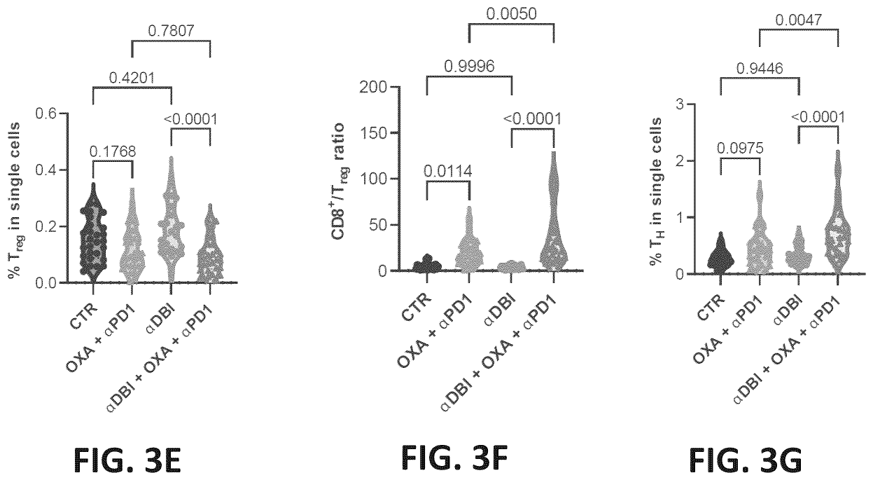

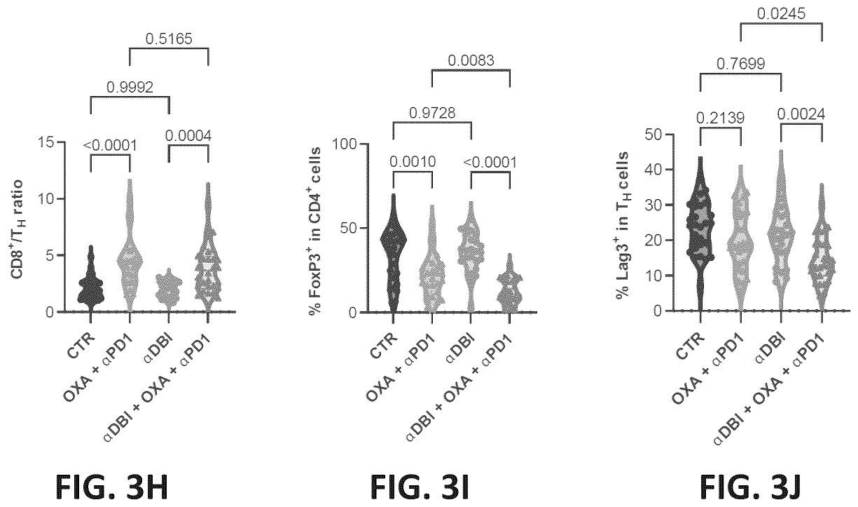

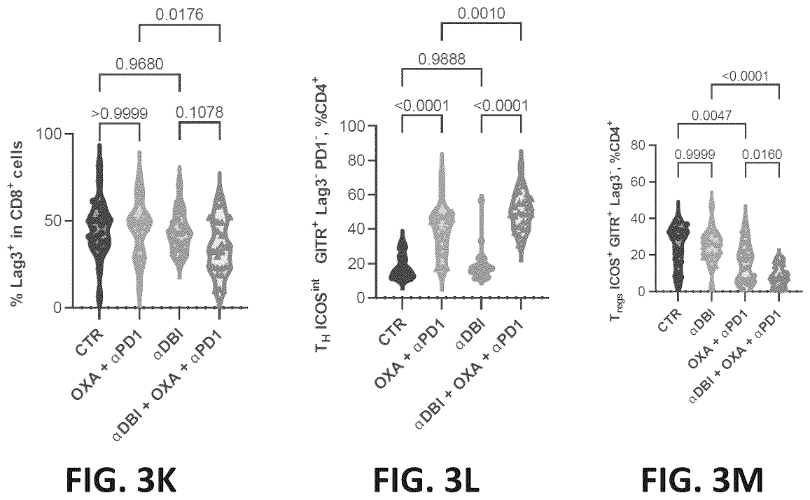

In another experiment following a similar treatment schedule, mice were sacrificed on day 18 post-inoculation (FIG. 3A), and the composition of the tumor immune infiltrate was determined by immunofluorescence staining followed by cytofluorometry (FIG. 3B-FIG. 3M). The anti-ACBP/DBI mAb amplified some effects of the chemoimmunotherapy (OXA+anti-PD1) on the cancer immune infiltrate such as the infiltration by CD4⁺ T cells (FIG. 3C), in particular T helper cells (T_{H}) defined as CD4⁺Foxp3⁻ cells (FIG. 3G). Anti-ACBP/DBI mAb improved the ratio of CD8⁺ T cells over regulatory T cells (T_{reg}) defined as CD4⁺Foxp3⁺ cells (FIG. 3F) and reduced the frequency of T_{reg} cells among CD4⁺ T cells (FIG. 3I). Anti-ACBP/DBI mAb reduced the proportion of T_{H} and CD8⁺ T cells that express the exhaustion marker LAG3 (FIG. 3J and FIG. 3K). Moreover, shifts in the activation/exhaustion markers ICOS and GITR were observed among T_{H} and T_{reg} cells (FIG. 3L and FIG. 3M). All these effects of anti-ACBP/DBI mAb were only detected in the context of chemo-immunotherapy, not in its absence (FIG. 3B-FIG. 3M). Altogether, these results suggest that anti-ACBP/DBI mAb improves therapy-induced immunosurveillance.

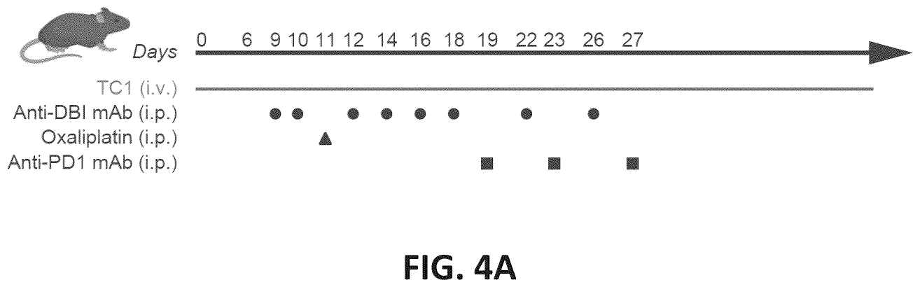

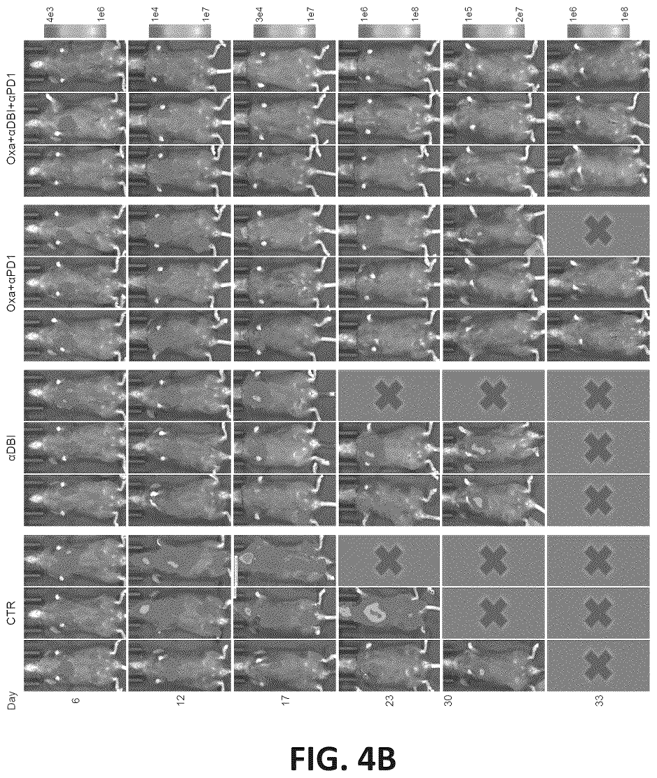

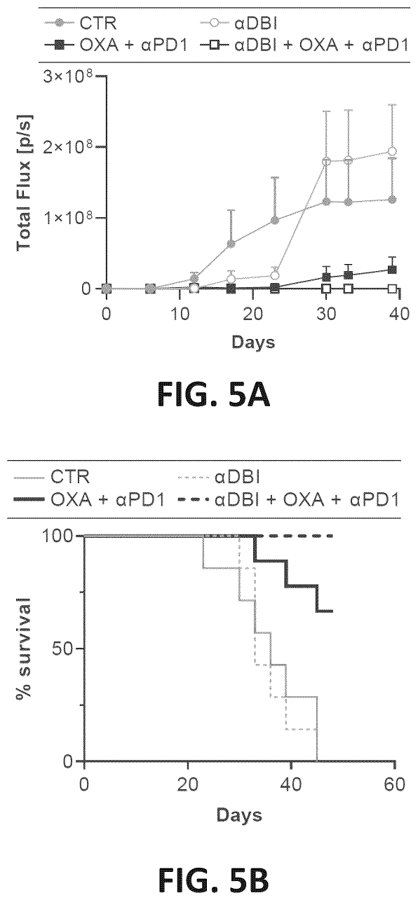

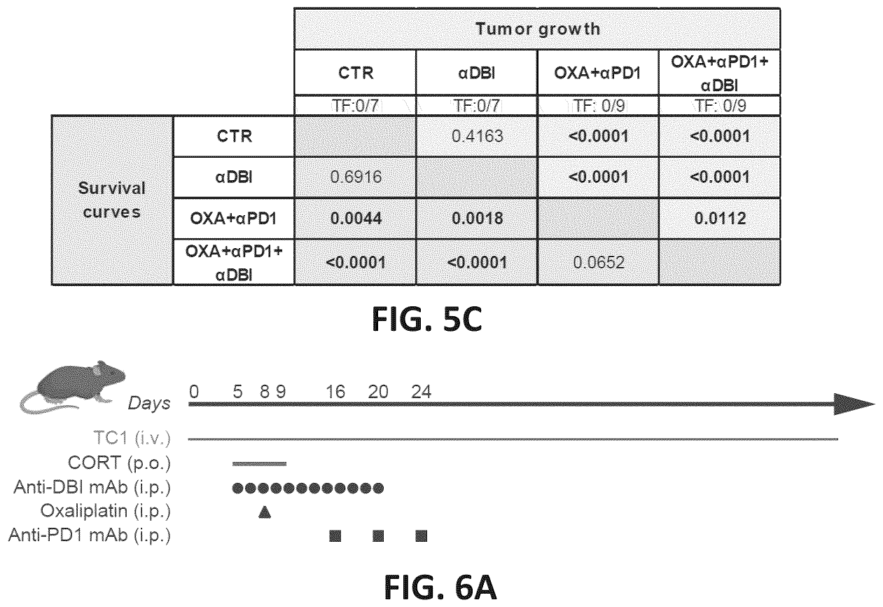

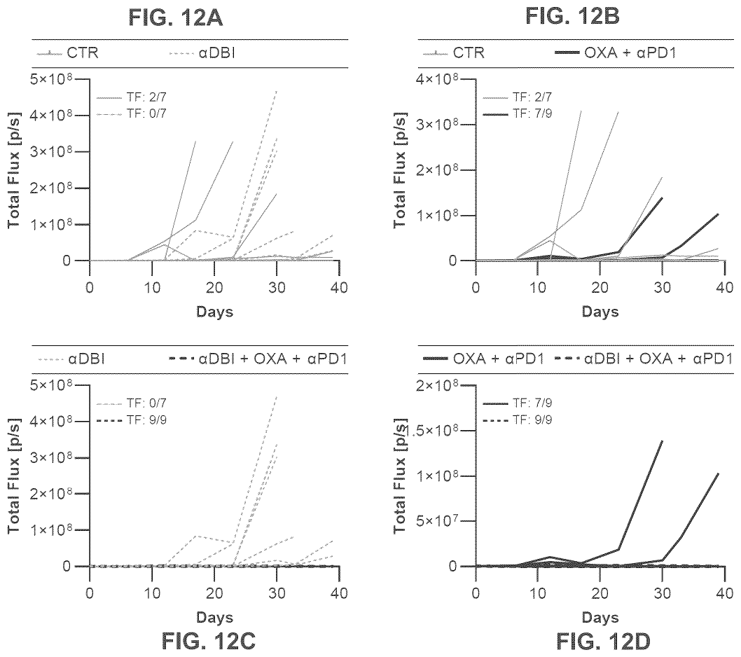

Next, the positive effects of anti-ACBP/DBI mAb on chemo-immunotherapy in another model, namely non-small cell lung cancers (NSCLC) were confirmed. TC1 NSCLC cells expressing luciferase injected intravenously (i.v.) into syngeneic immunocompetent C57BL/6J mice form orthotopic lung cancers that can be monitored by bioluminescence imaging. [Liu, P. et al., Crizotinib-induced immunogenic cell death in non-small cell lung cancer, Nature Communications, 2019, 10 (1).] From day 9 post-inoculation of TC1 cells, NSCLC-bearing mice were periodically i.p. injected with anti-ACBP/DBI mAb (or an isotype control mAb) and then were treated on day 11 post-inoculation with OXA (i.p. once) and anti-PD1 mAb (injected i.p. on days 19, 23 and 27 post-TC1 injection) (FIG. 4A) and tumor progression was determined by bioluminescence imaging (FIG. 4B). Anti-ACBP/DBI mAb improved the tumor control achieved by chemo-immunotherapy (FIG. 5A and FIG. 12A-FIG. 12D). These results corroborate the cancer treatment-improving effects of ACBP/DBI neutralization.

### Improvement of Chemo-Immunotherapy Outcome by ACBP DBI Neutralization in the Context of Corticotherapy

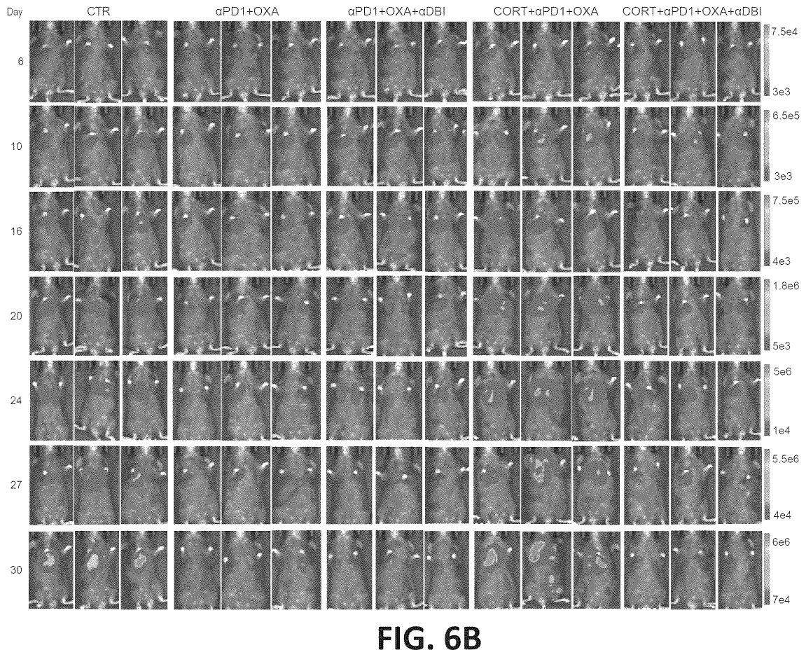

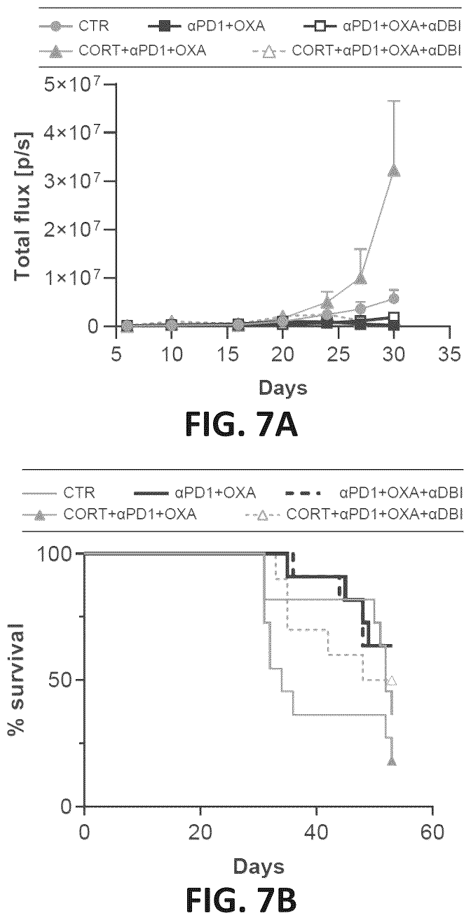

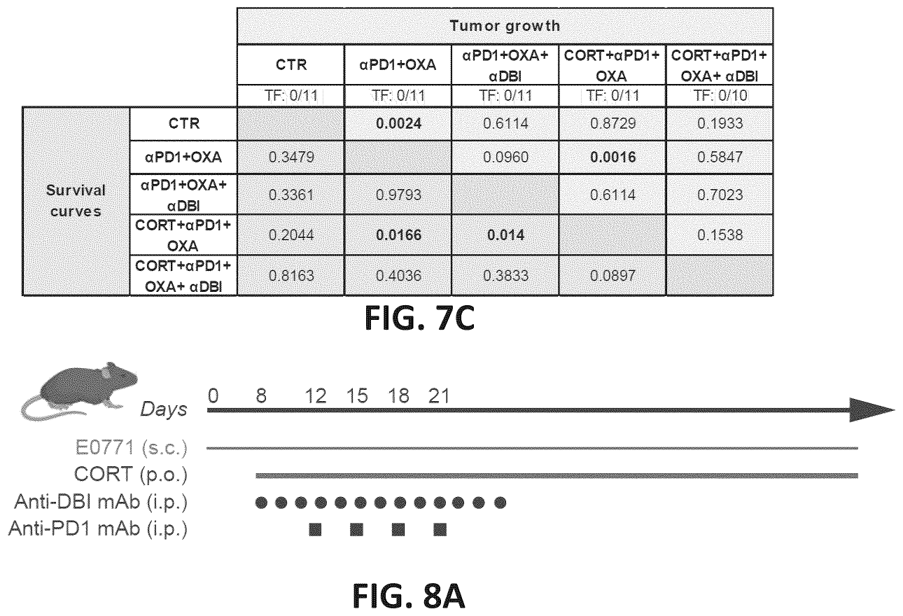

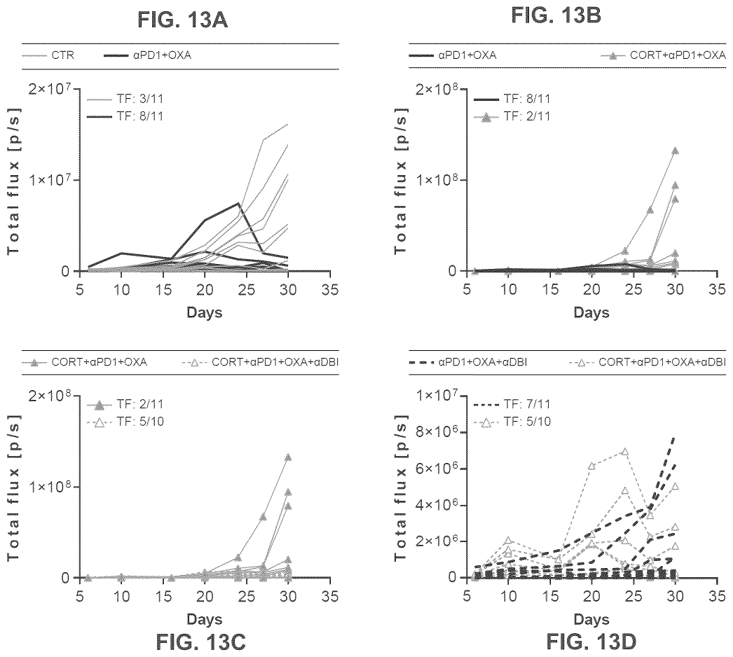

In the next experiment, five days after injection of the TC1 cells, animals were optionally treated with corticosterone (CORT) or as a control with vehicle only (0.66% ethanol, which was permanently present in the drinking water from days 5 to 9) and/or anti-ACBP/DBI mAb (or the isotype control mAb). In addition, the animals received one single dose of oxaliplatin-based chemotherapy (i.p. injected once on day 8 post-TC1 injection, which is 3 days earlier than in preceding experiment), followed by PD-1-targeted immunotherapy (injected i.p. on days 16, 20 and 24 post-TC1 injection), i.e., 3 days earlier than in the preceding experiment (compare FIG. 5A and FIG. 6A). Of note, corticotherapy strongly interfered with this chemo-immunotherapeutic effect, which, however, was restored by injection of the anti-ACBP/DBI antibody (FIG. 6B, FIG. 7A and FIG. 13A-FIG. 13D). These data indicate that ACBP/DBI neutralization can reverse the immunosuppressive effect of corticotherapy that interferes with the anti-cancer effects of chemo-immunotherapy.

### Improvement of Immunotherapy Outcome by ACBP/DBI Neutralization in the Absence and Presence of Corticotherapy

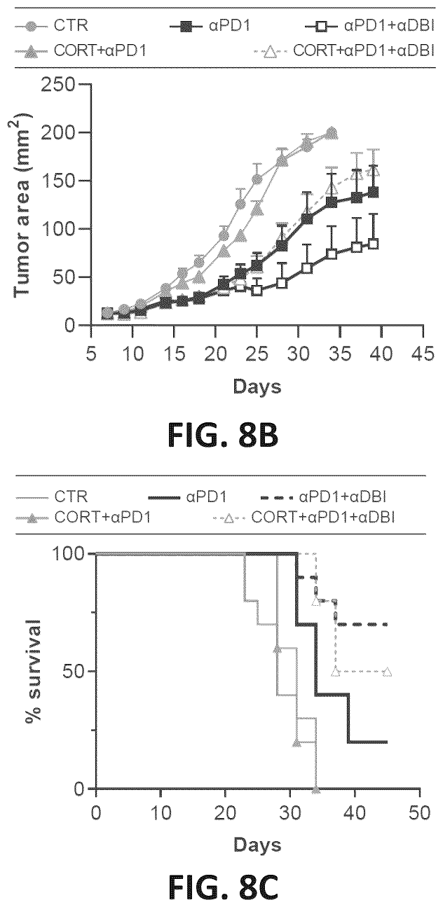

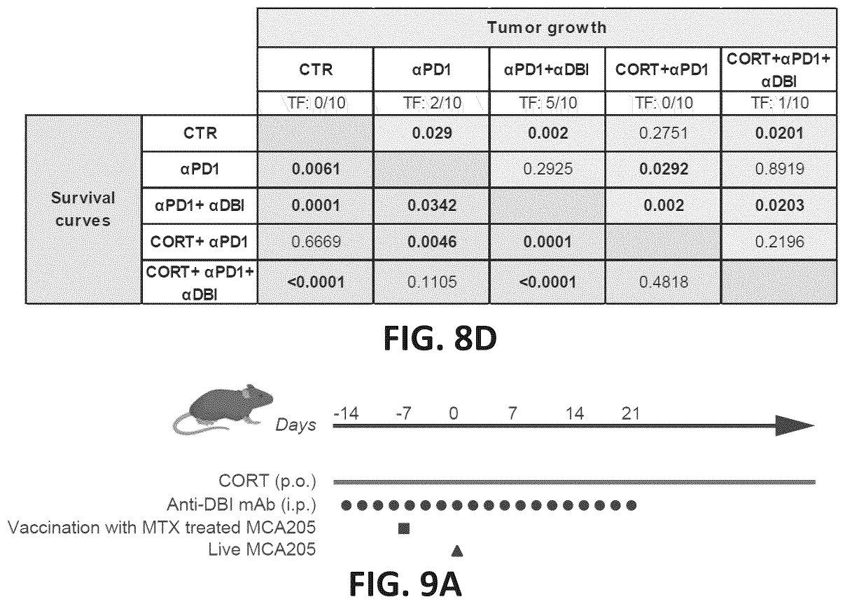

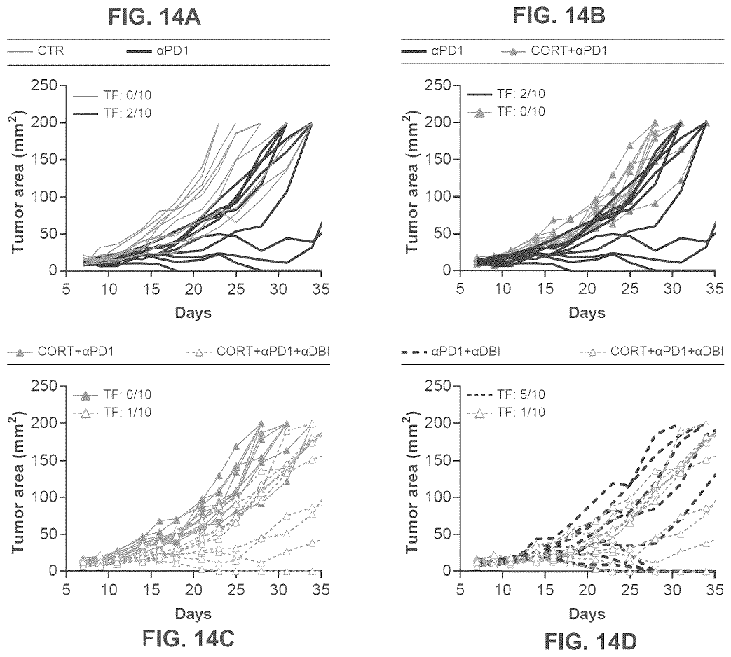

C57BL/6J mice were inoculated subcutaneously (s.c.) with syngeneic E0771 breast cancer cells. Six days later treatment of corticosterone (CORT or as a control with vehicle only, 0.66% ethanol, which was administered in the drinking water) and anti-ACBP/DBI mAb (or the isotype control mAb, both injected i.p. three times per week) was initiated and tumor growth was monitored over several weeks. On days 12, 15, 18 and 21, the animals received intraperitoneal (i.p.) injections of a PD-1-specific mAb (or its isotype control) (FIG. 8A). This treatment normally reduces tumor growth [Zheng, X. et al., Increased vessel perfusion predicts the efficacy of immune checkpoint blockade, J Clin Invest, 2018, 128(5), pp. 2104-2115.] but this effect was lost in CORT-treated animals unless they received anti-ACBP/DBI mAb (FIG. 8B). The efficacy of immunotherapy with anti-PD1 mAb, its loss with corticotherapy, as well as its restoration by anti-ACBP/DBI mAb was detectable at the level of tumor growth and animal survival (FIG. 8B and FIG. 14A-FIG. 14D). Moreover, anti-ACBP/DBI mAb significantly improved the immunotherapeutic effect of αPD-1 at the level of animal survival, even in the absence of corticotherapy (FIG. 8C and FIG. 8D). These findings indicate that ACBP/DBI neutralization can improve the outcome of immunotherapy both in the absence and in the presence of corticotherapy.

### Improvement of the Anti-Cancer Immune Response by ACBP DBI Neutralization in the Context of Corticotherapy

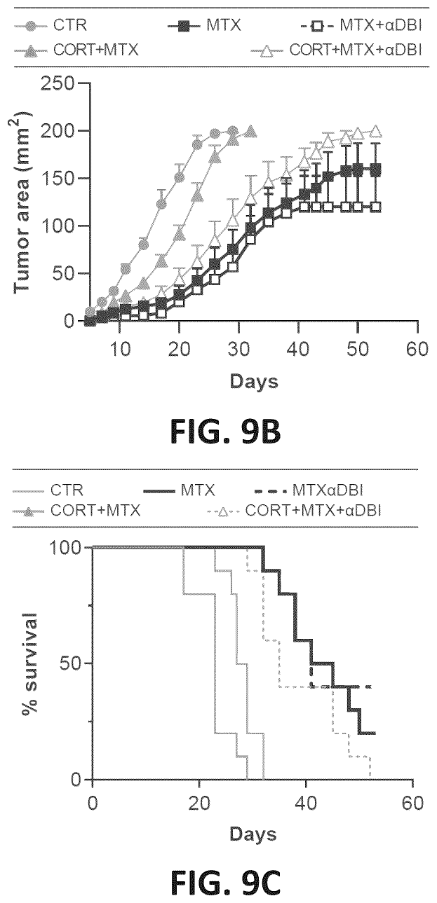

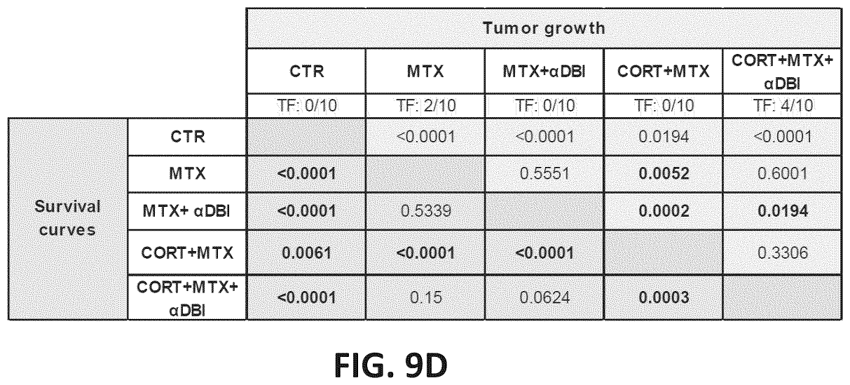

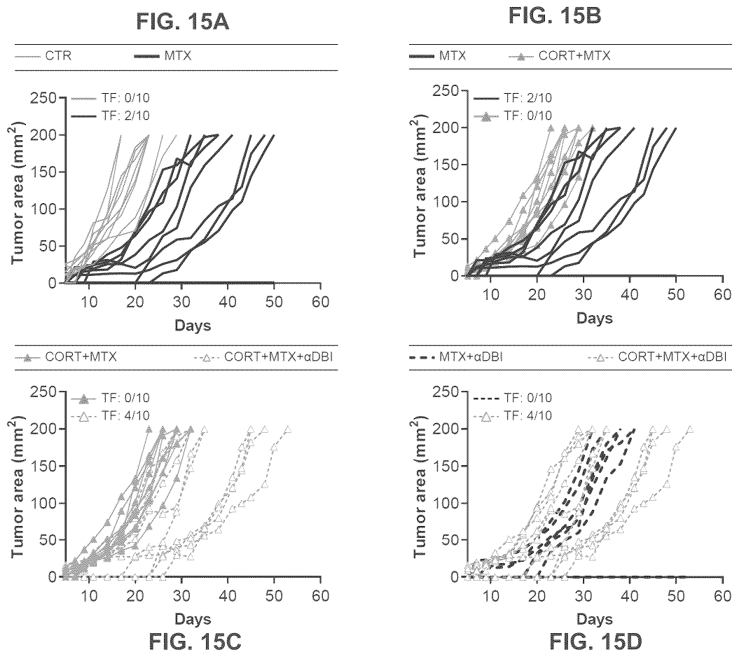

C57BL/6J mice were treated with the glucocorticoid corticosterone (CORT or as a control with vehicle only, 0.66% ethanol, which was administered in the drinking water) and an anti-ACBP/DBI monoclonal antibody 7A (mAb or its isotype IgG2a control), which was injected intraperitoneally (i.p.) three times per week, as indicated in the scheme (FIG. 9A). After the first week of treatment, the animals were subcutaneously (s.c.) injected with mitoxantrone (MTX)-treated MCA205 fibrosarcoma cells, knowing that MTX-induces ICD and that MTX-treated cancer cells induce a protective anti-cancer immune response that suppresses, delays or reduces the growth of tumors that would grow in naïve mice after injection of live, untreated MCA205 fibrosarcoma cell. [Panaretakis T. et al., Mechanisms of pre-apoptotic calreticulin exposure in immunogenic cell death, EMNO J, 2009, 28 (5), pp. 578-590.] Hence, if injected into the opposite flank 1 week after the vaccination with MTX-treated MCA205 cells, live MCA205 fibrosarcoma cells grew less and were compatible with longer animal survival than if such live cells were inoculated into naïve mice. Treatment with CORT reduced this vaccination effect, which, however, was largely recovered by the anti-ACBP/DBI mAb (FIG. 9B and FIG. 15A-FIG. 15D). In conclusion, it appears that ACBP/DBI neutralization can reverse the immunosuppressive effects of corticotherapy on the anti-cancer immune response.

### Example 2: Role of DBI in Hepatocellular Carcinogenesis

The implications of DBI in malignant disease are poorly studied and there is no knowledge on the role of DBI in HCC. However, Applicants surprisingly found, using a compendium of methods to inhibit DBI (e.g., by its knockout, receptor mutation or antibody-mediated neutralization), a critical implication of DBI in hepatocarcinogenesis and tumor progression. Of note, high intratumoral DBI mRNA expression and high levels of circulating DBI protein levels are associated with features of poor prognosis in HCC patients, supporting the translational relevance of these findings.

To investigate its role in hepatocarcinogenesis in mice, Applicant inhibited DBI using three methods, namely (i) inducible whole-body knockout of DBI, (ii) a point mutation of the DBI receptor (GABRG2) or (iii) induction of autoantibodies neutralizing DBI. Applicant found that DBI played a major pro-carcinogenic role in HCC induced by intrahepatic transplantation of HCC cell lines, transgenic co-expression of the two oncogenes Myc and Ctnnb1, as well as chronic challenge with a Western style diet together with either CCl₄ or diethylenenitrosamine. Inhibition of DBI largely normalized HCC-associated gene expression, thus reducing oncogenic alterations in cell cycle-relevant, immunosuppressive and ferroptosis-regulatory genes. Functional experimentation confirmed that DBI inhibition reduced the proliferation of normal and malignant hepatocytes, increased HCC responses to PD-1 blockade, and sensitized HCC to the therapeutic induction of ferroptosis. Without wishing to be bound by theory, DBI constitutes an actionable target involved in HCC pathogenesis.

### Increased DBI Levels in Human HCC

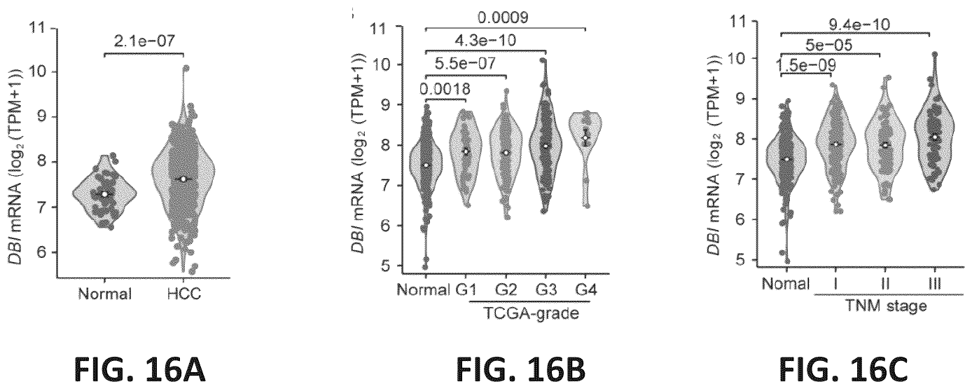

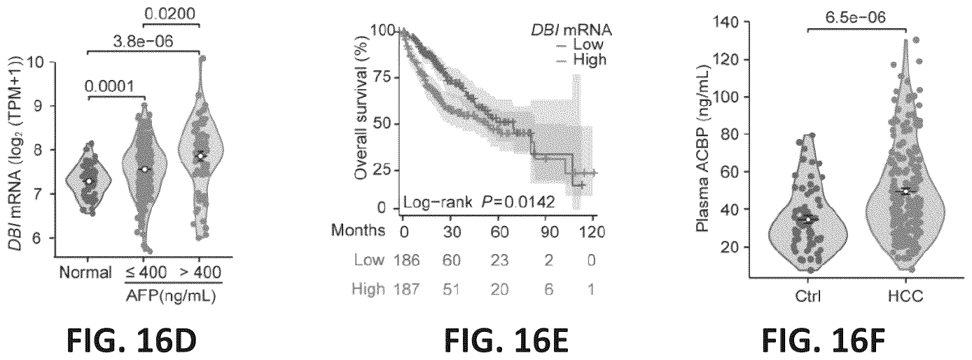

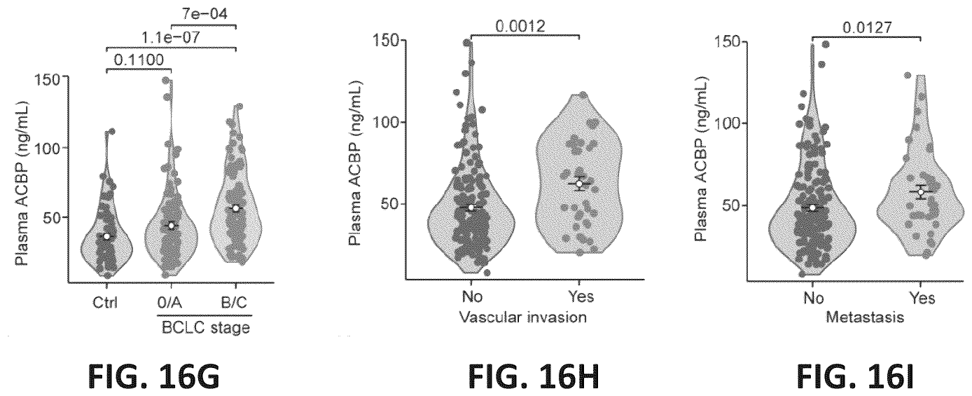

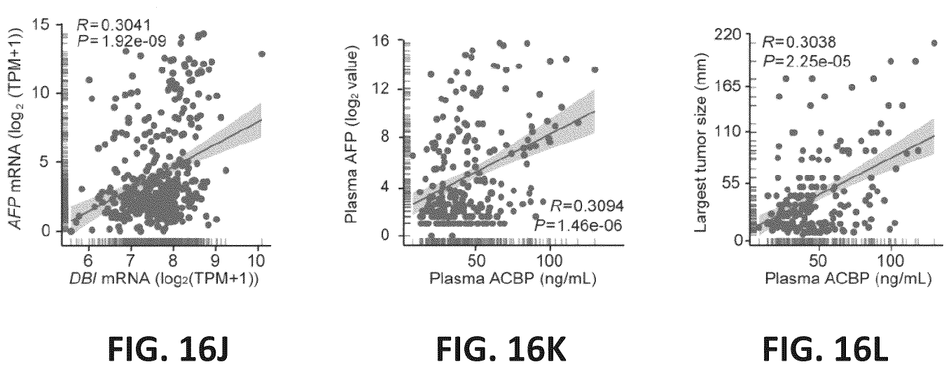

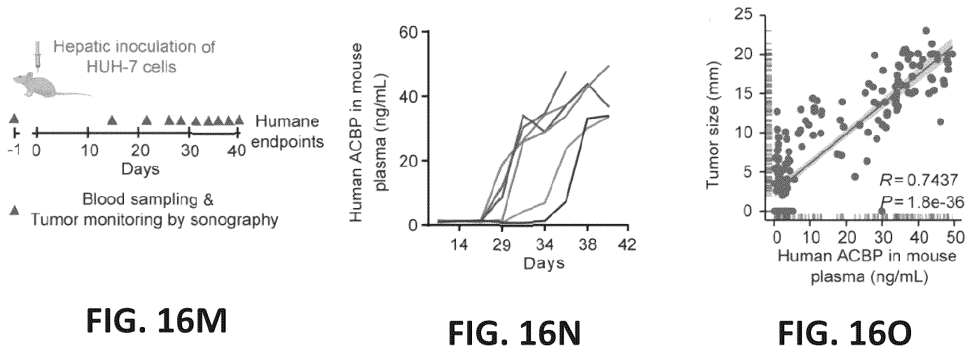

In 9 different publicly available gene expression datasets including The Cancer Genome Atlas (TCGA), HCC tumors consistently contain higher DBI mRNA levels than the normal adjacent liver tissue (FIG. 16A). According to the TCGA, this DBI elevation occurred independently from each of the components of the tumor/node/metastasis (TNM) staging system, Child-Pugh (CP) grade, tumor differentiation, vascular invasion, as well as levels of fibrosis and inflammation (FIG. 16B and FIG. 16C). However, DBI mRNA was particularly abundant in patients with alpha-fetoprotein (AFP) protein levels >400 ng/mL (FIG. 16D) and DBI mRNA levels above the median values were associated with shorter overall survival in TCGA (FIG. 16E).

Using ELISA, DBI protein levels were measured in the plasma of 146 HCC patients belonging to different BCLC-stages and 58 plasmas of patients with chronic liver diseases without any history of HCC collected at a university hospital (AP-HP, Bobigny, France). ELISA-detectable plasma DBI levels (ng/ml) were found to be higher in HCC patients compared with the group without any current or prior HCC (FIG. 16F), correlating with poor-prognosis features such as advanced BCLC stage (FIG. 16G), vascular invasion (FIG. 16H) and extrahepatic metastasis (FIG. 16I). In addition, DBI mRNA correlated with AFP mRNA in liver biopsies (FIG. 16J), echoing a correlation between plasma levels of DBI protein and AFP protein (FIG. 16K). Moreover, DBI plasma concentrations correlated with tumor size, as detected by CT scans (FIG. 16L). Altogether these results support the idea that advanced HCC is associated with high DBI levels.

Importantly, when human HCC cells were orthotopically inoculated into the livers from immunodeficient mice (FIG. 16M), human but not mouse DBI plasma concentrations measured with species-specific ELISAs (that distinguish human and mouse DBI) gradually increased over time (FIG. 16N), correlating with the size of the tumors (FIG. 16O). These findings indicate that raising circulating DBI may directly stem from HCC. Without wishing to be bound by theory, altogether, these findings suggest that HCC progression is associated with an increase in DBI expression by malignant tissues as well as by an increase in DBI plasma levels.

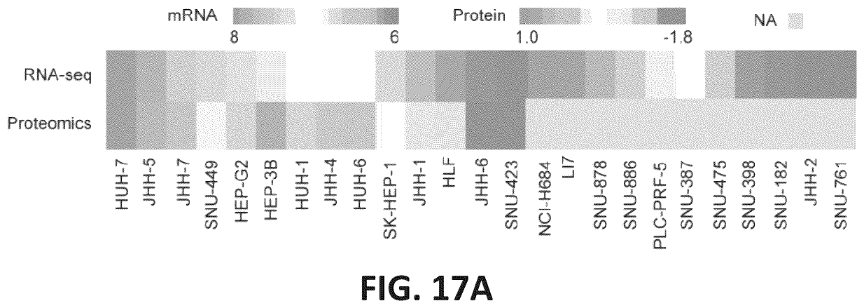

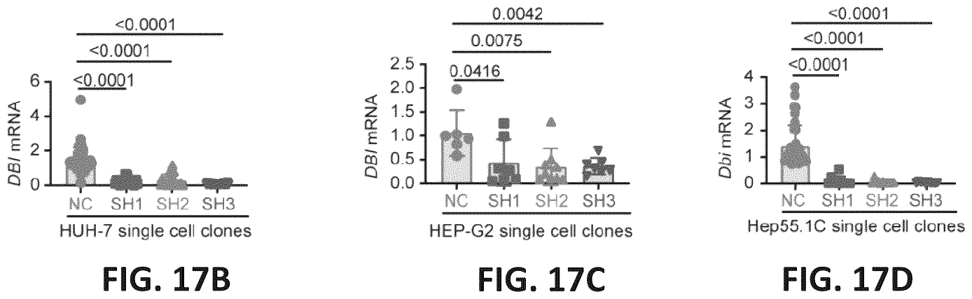

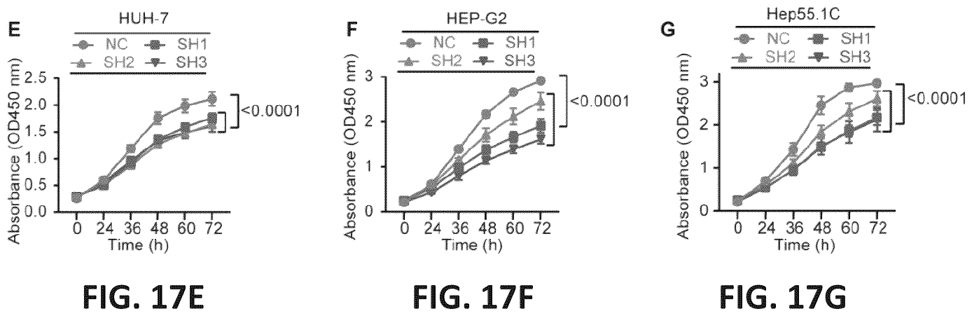

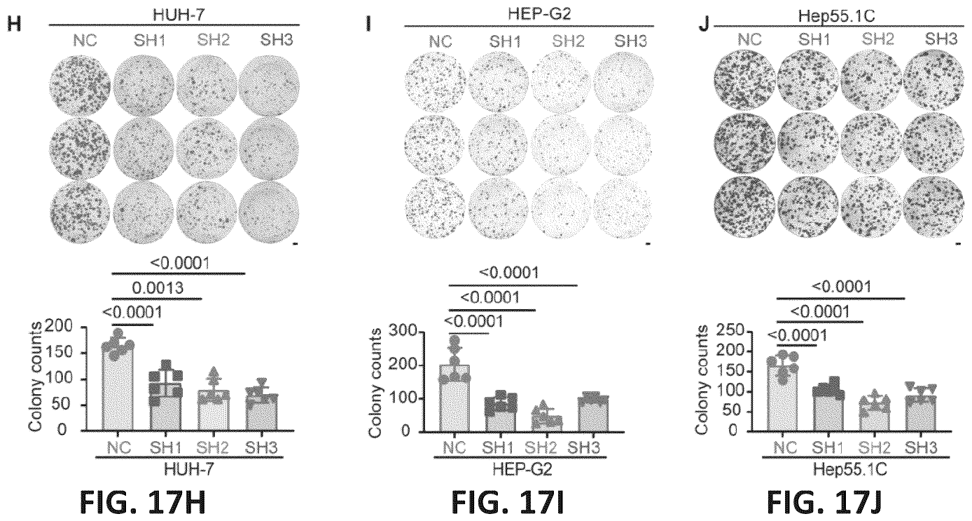

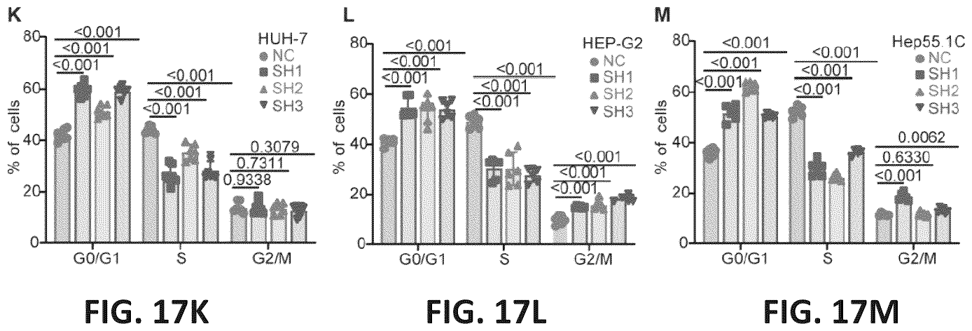

For this reason, the potential pathogenic role of DBI in HCC was investigated in suitable preclinical models. Intra- and extra-cellular DBI contribute to the pathogenesis of transplantable, oncogene- and carcinogen-induced HCC. Among a collection of distinct human HCC cell lines, HUH-7 cells express the highest DBI mRNA and protein levels (FIG. 17A). Knockdown of DBI with three different shRNAs yielded HUH-7 clones that exhibited less proliferation and clonogenic potential, as well as a reduced proportion of cells in the S-phase of the cell cycle. Similar results were obtained for human hepatoblastoma HEP-G2 cells and mouse HCC Hep55.1C cells subjected to the knockdown of DBI and Dbi, respectively (FIG. 17B-FIG. 17M).

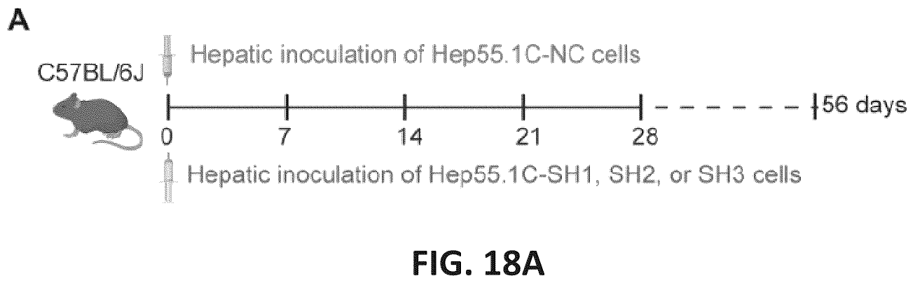

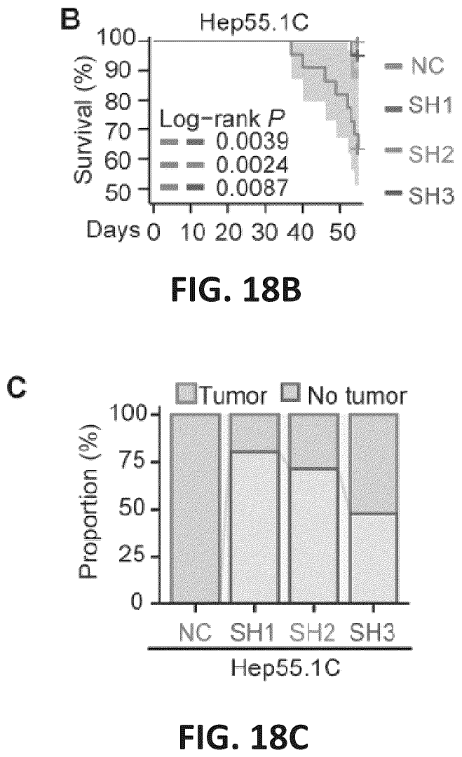

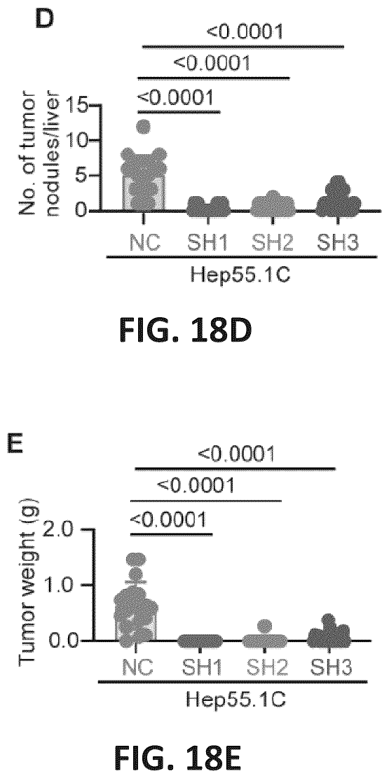

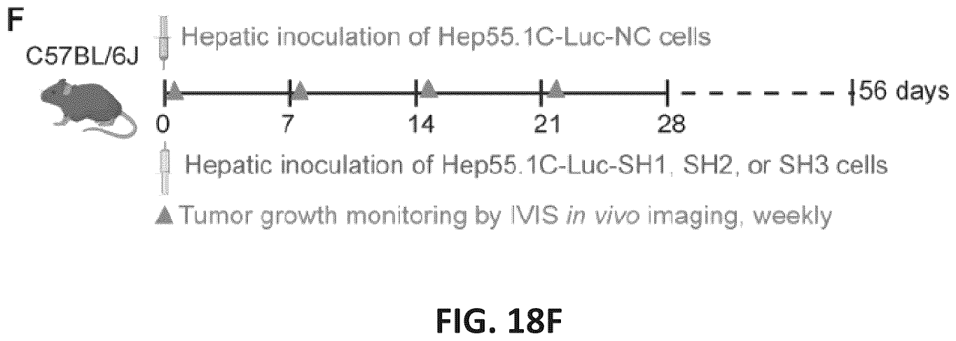

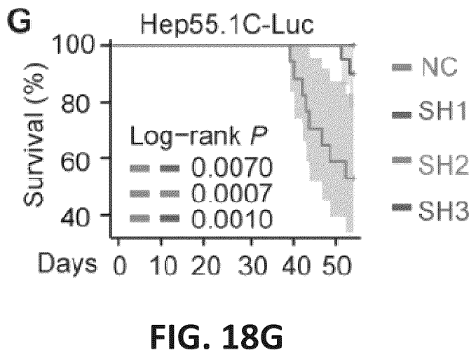

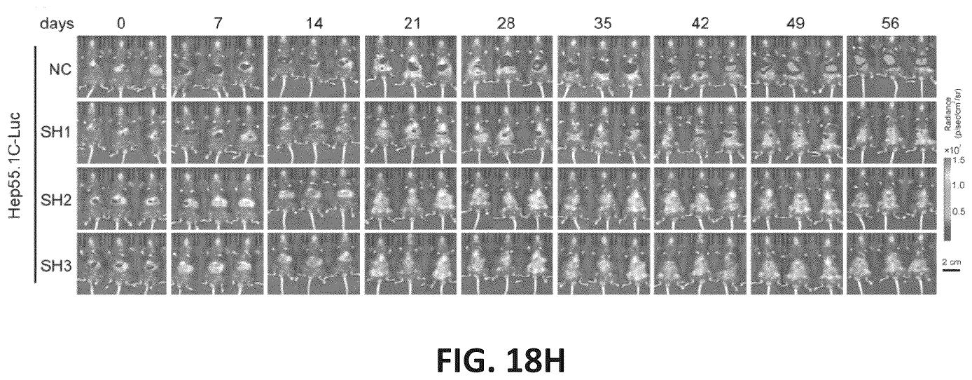

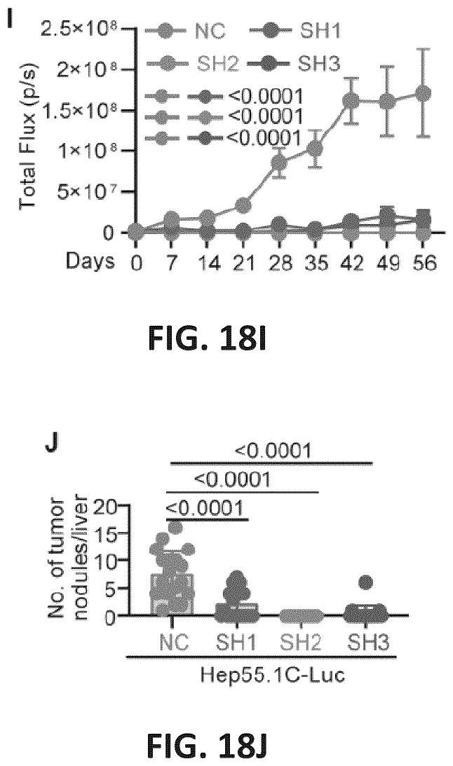

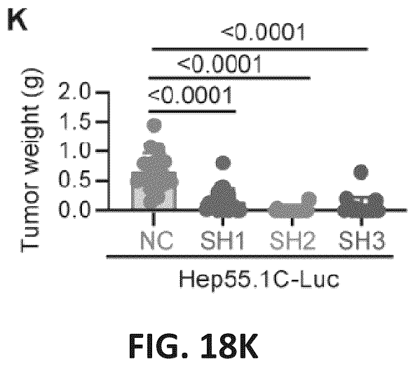

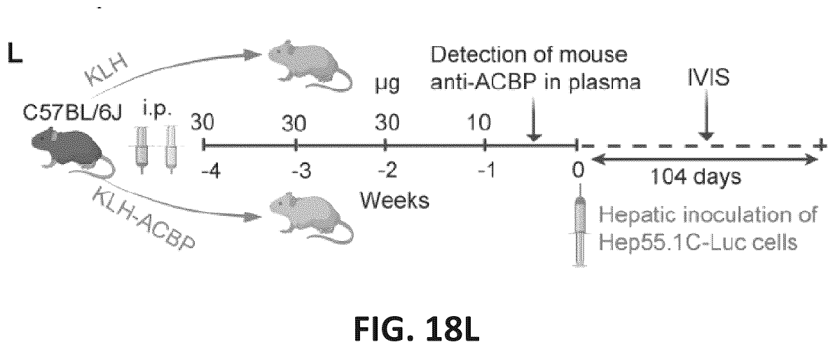

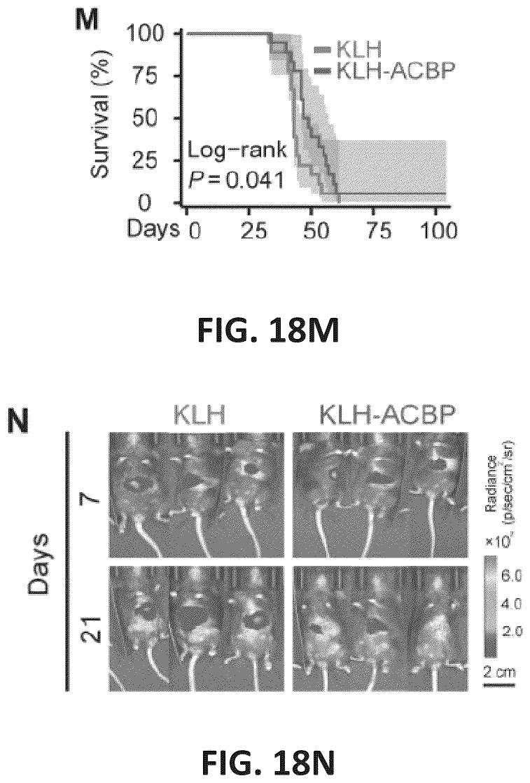

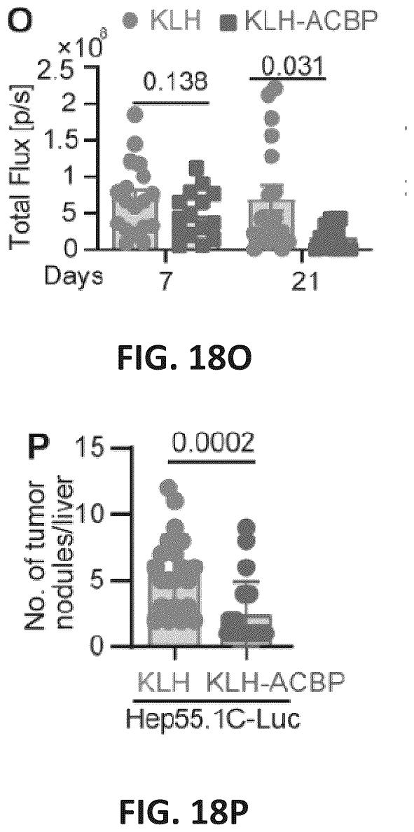

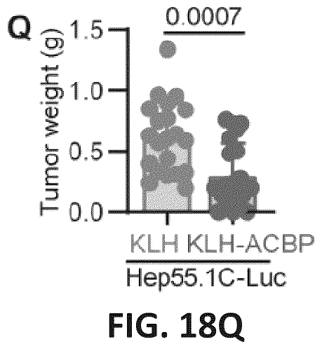

As compared to the parental cell line, three Hep55.1C clones subjected to DBI depletion (by three distinct shRNAs) were relatively poorly pathogenic when inoculated orthotopically into the livers of immunocompetent C57BL/6 mice (FIG. 18A), as indicated by reduced mortality of recipient mice (FIG. 18B), a diminished number of mice developing macroscopic HCC (FIG. 18C), as well as a decreased number and total weight of HCC nodules at endpoint (FIG. 18D and FIG. 18E). Constant monitoring of tumor growth using luciferase-transduced Hep55.1C clones confirmed the failure of Dbi-depleted cells to develop HCC (FIG. 18F-FIG. 18K). Of note, neutralization of extracellular DBI protein by means of a suitable auto-vaccination protocol (in which autoantibodies against DBI were induced by adjuvanted inoculation of DBI conjugated to the immunogen keyhole limpet hemocyanine, KLH) also delayed the orthotopic growth of parental (DBI-expressing) luciferase-transduced Hep55.1C cancers as compared to control animals immunized with KLH alone (FIG. 18L-FIG. 18Q).

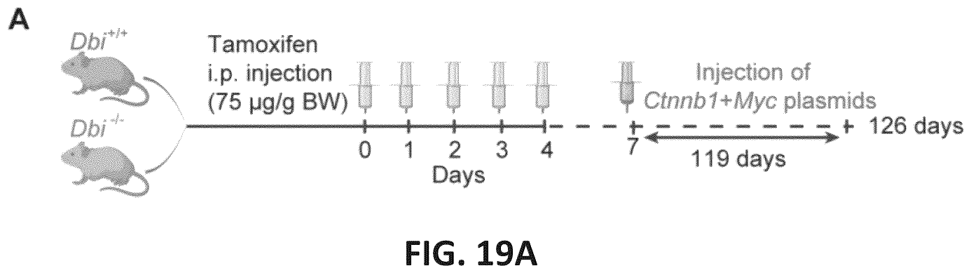

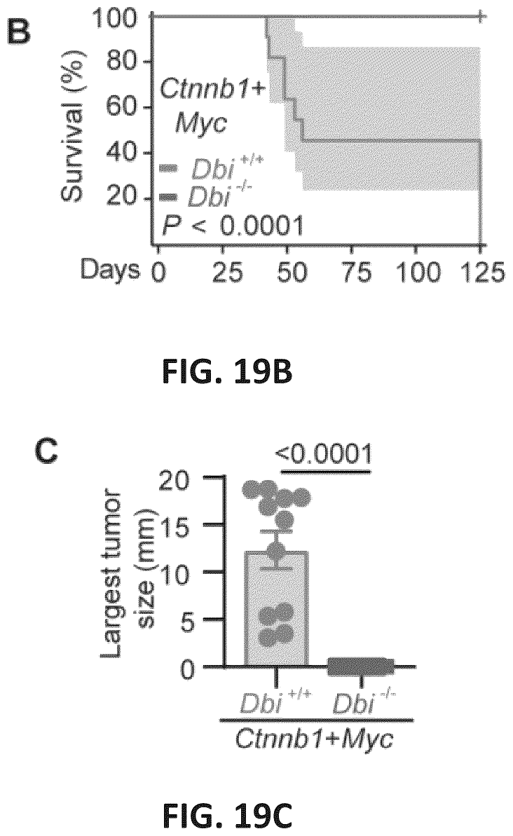

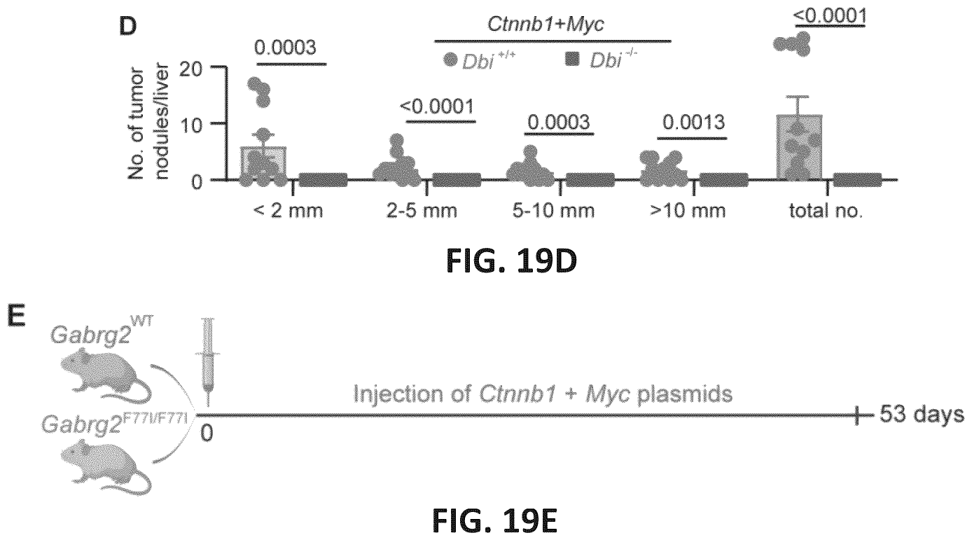

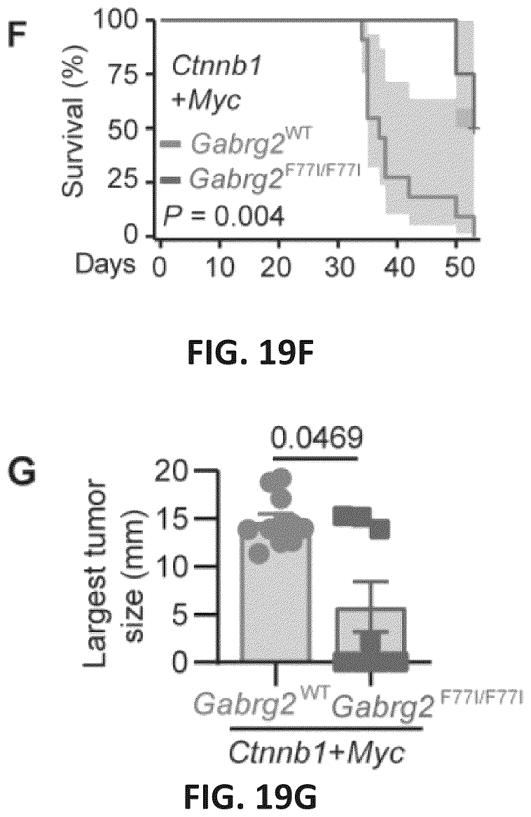

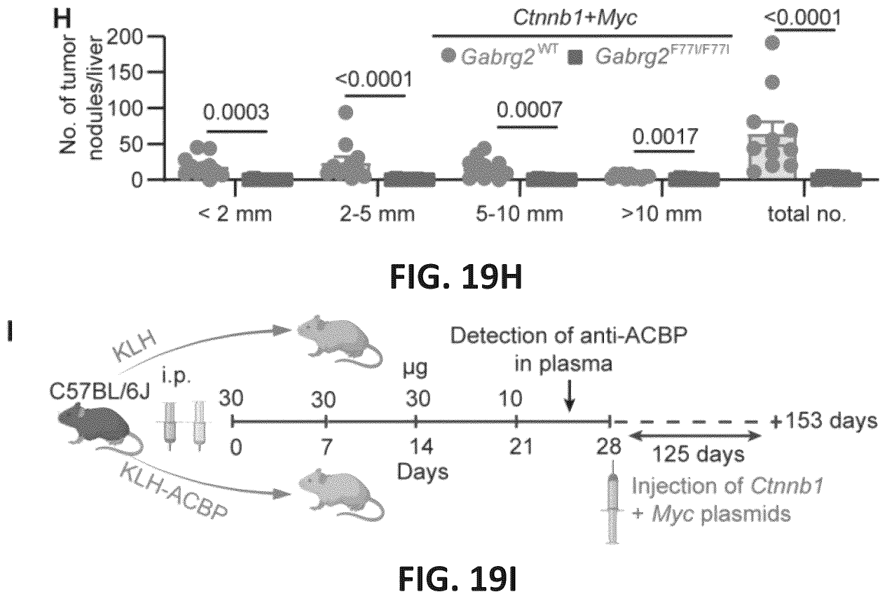

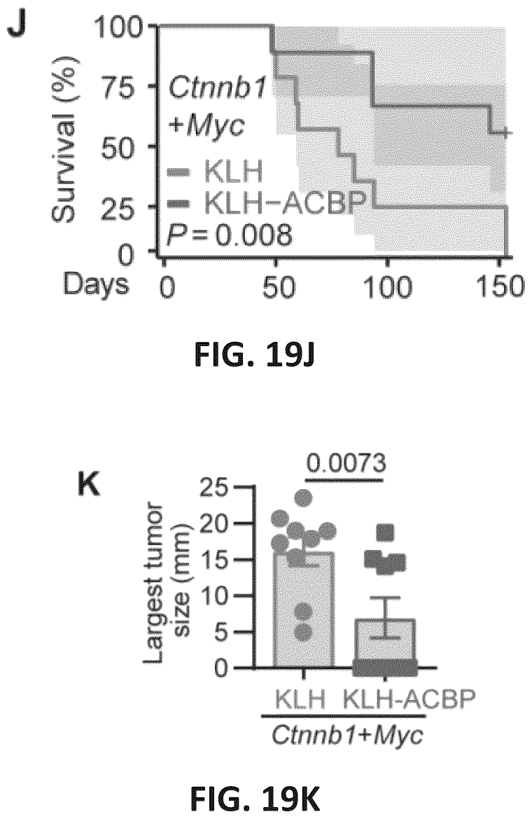

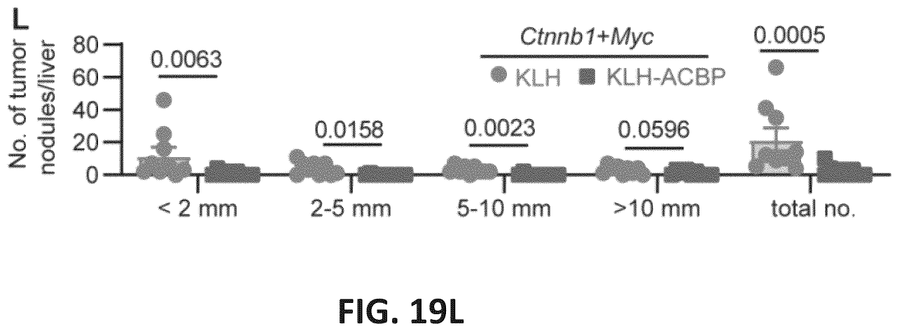

Next a model of oncogene-induced hepatocarcinogenesis in which two plasmids coding for Myc and Ctnnb1 were co-transfected into hepatocytes by hydrodynamic injection was studied. Mice in which a floxed version of DBI (DBI^{f/f}) was ubiquitously excised by a tamoxifen-inducible Cre recombinase (UBCcre/ERT2) (genotype; UBCcre/ERT2::DBI^{f/f}, Dbi^{+/+}, control; DBI^{f/f} without CRE, Dbi^{-/-}) were rather resistant to Myc/Ctnnb1-induced hepatic carcinogenesis (FIG. 19A-FIG. 19D). Moreover, mice in which the γ2 subunit of GABAA receptor (Gabrg2) was homozygously mutated (F77I) (genotype: Gabrg2^{F77I/F77I}), causing a loss of interaction with DBI, were relatively resistant to Myc/Ctnnb1-induced HCC as well (FIG. 19E-FIG. 19H). Finally, immunization with KLH-DBI conferred partial protection against Myc/Ctnnb1-mediated hepatocellular carcinogenesis (FIG. 19I-FIG. 19L).

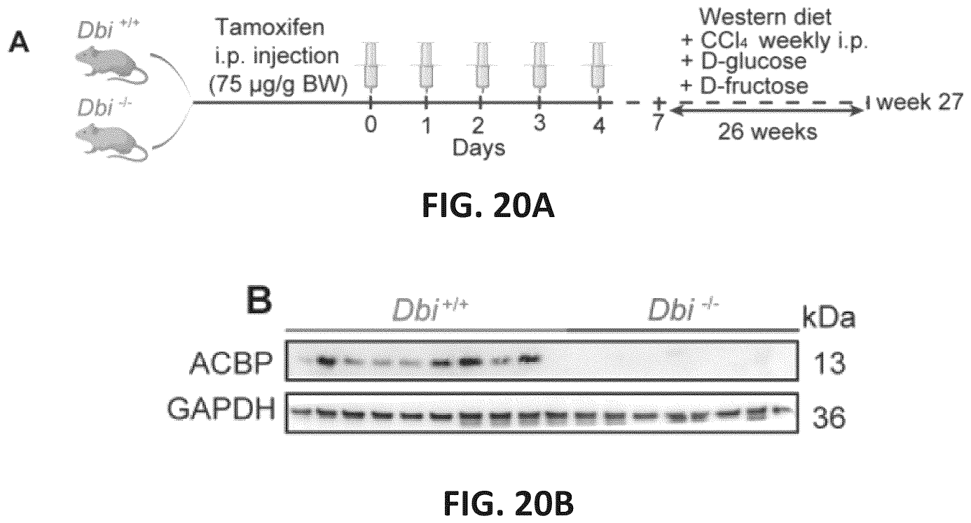

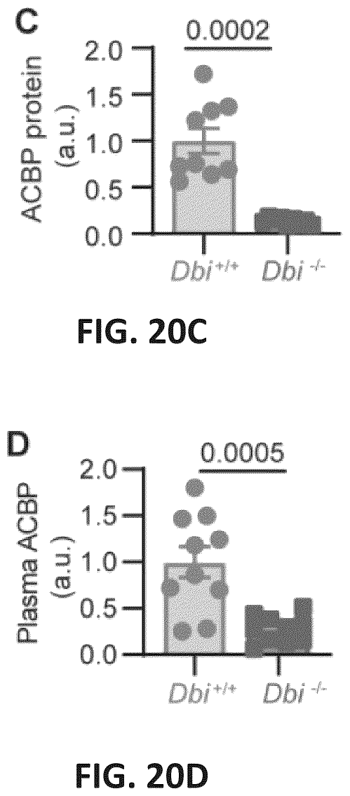

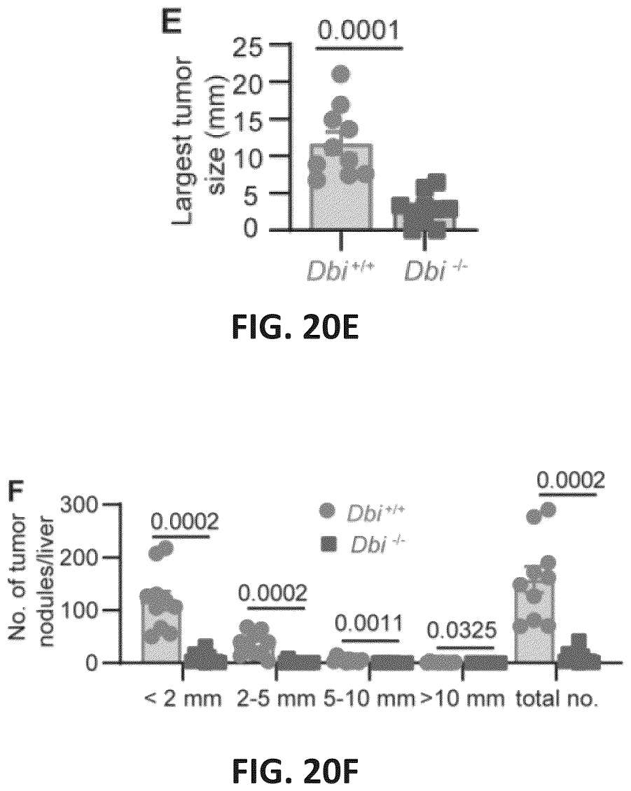

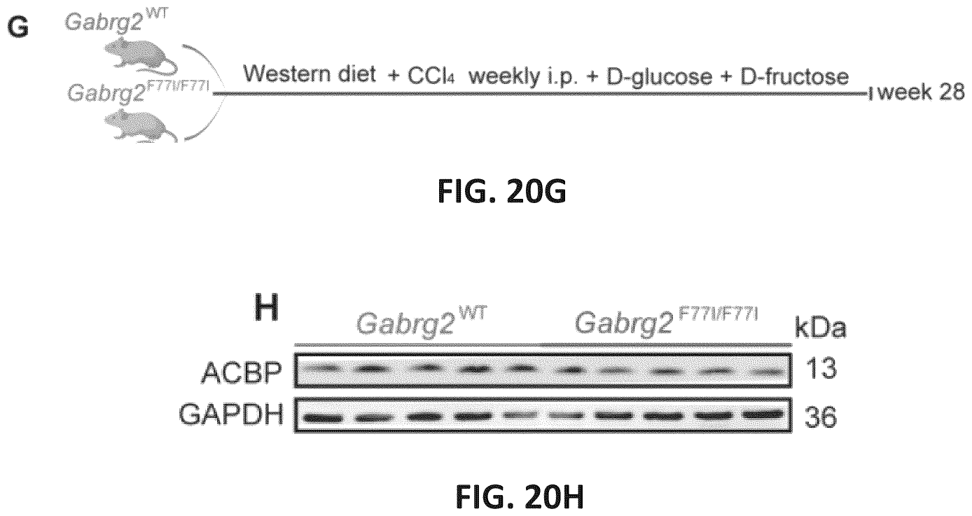

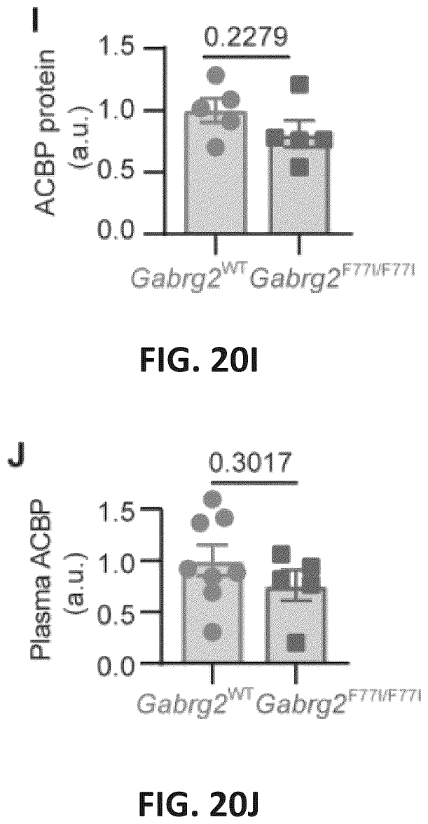

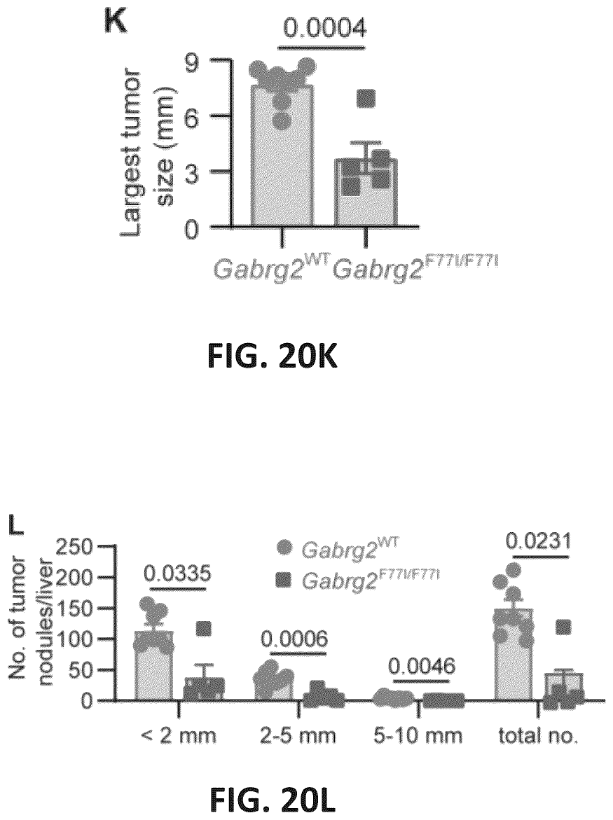

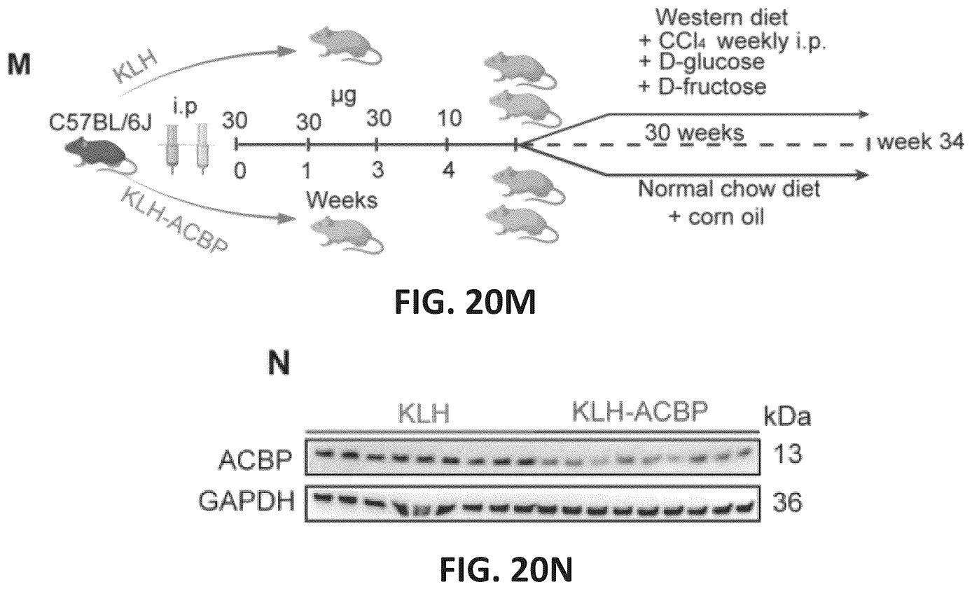

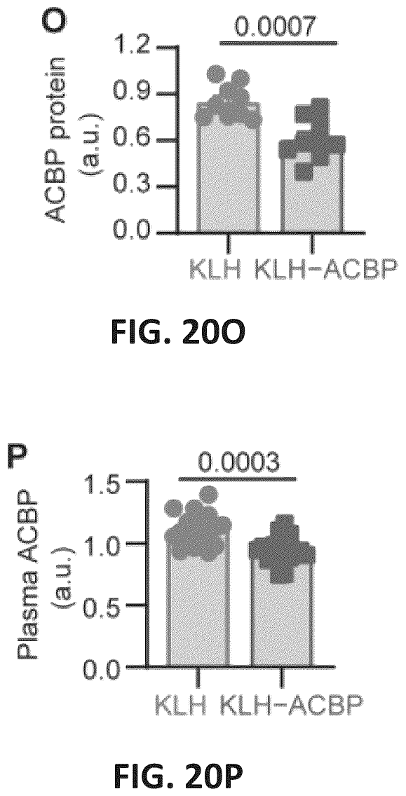

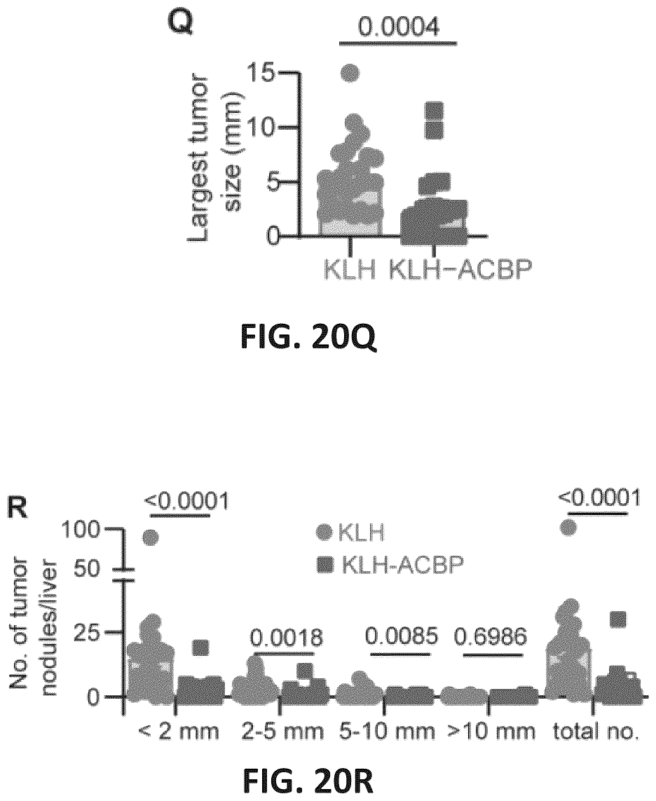

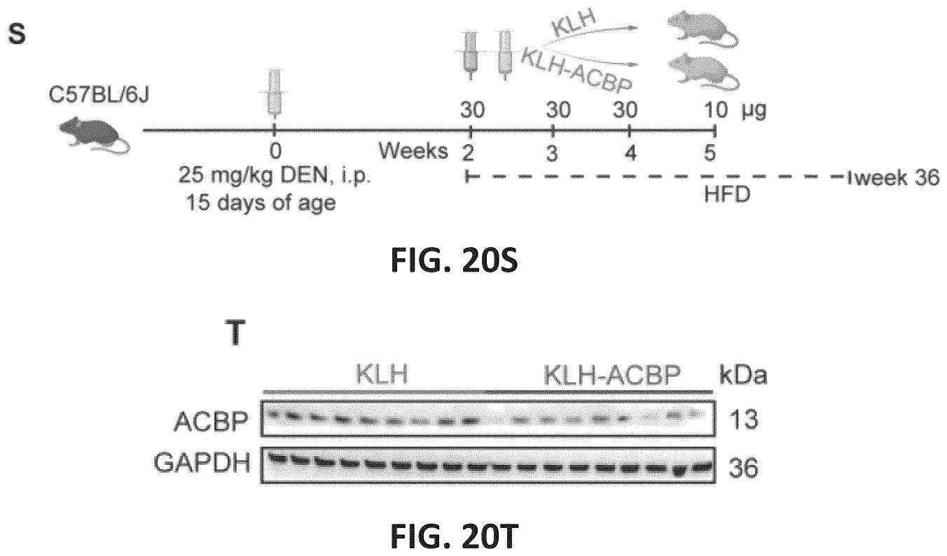

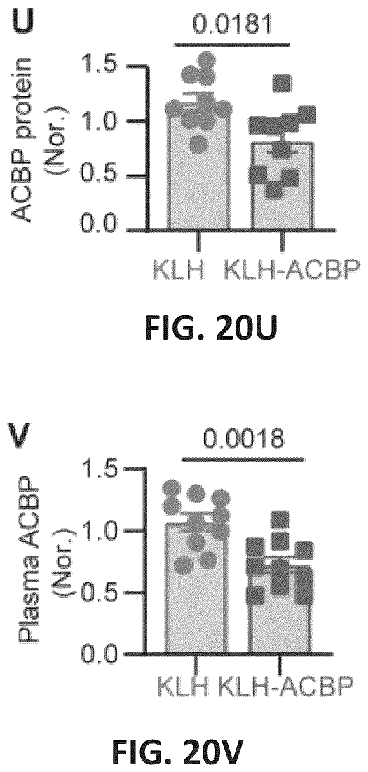

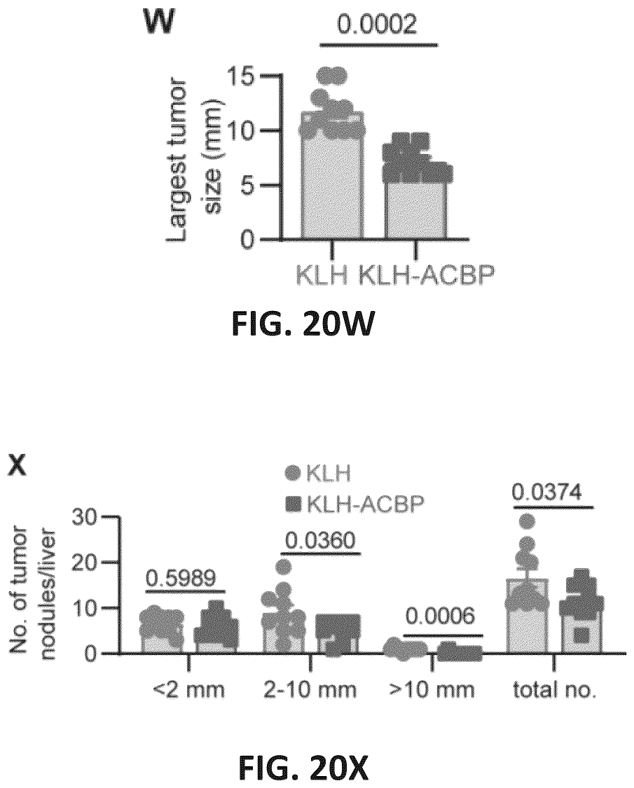

As the possible implication of DBI in diet- and toxin-induced HCC is elusive, a model of hepatic carcinogenesis induced by a Western style diet (WD) combined with weekly i.p. injections of the hepatotoxin CCl₄ was examined. Knockout of Dbi (FIG. 20A-FIG. 20F), homozygous Gabrg2 mutation (FIG. 20G-FIG. 20L), as well as immunization with KLH-Dbi (FIG. 20M-FIG. 20R), mitigated the stigmata of WD+CCl₄-induced liver damage (non-alcoholic steatohepatitis [NASH], lobular inflammation, ballooning with Mallory-Denk bodies and fibrosis), reduced liver Dbi mRNA and protein expression as well as circulating DBi concentrations, and inhibited the development of HCC. Thus, all protocols of DBI inhibition reduced the size and number of WD/CCl₄-induced tumor lesions developing in the liver (FIG. 20E, FIG. 20F, FIG. 20K, FIG. 20L, FIG. 20Q, and FIG. 20R). Similar anti-NASH, anti-fibrotic and oncosuppressive effects were observed for KLH-Dbi vaccination (as compared to KLH-only-vaccinated controls) when the disease was induced by a combination of HFD and the carcinogen diethylnitrosamine (DEN) (FIG. 20S-FIG. 20X).

Altogether these results indicate that hepatic carcinogenesis is favored by DBI. This effect of DBI is at least partially mediated by the extracellular pool of DBI acting on GABAA receptors.

### DBI Neutralization Improves Immunosurveillance

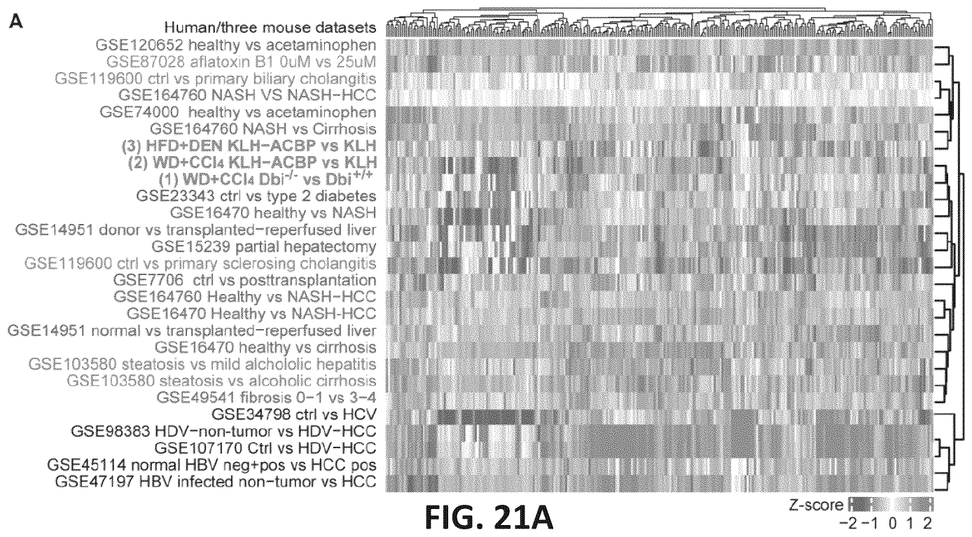

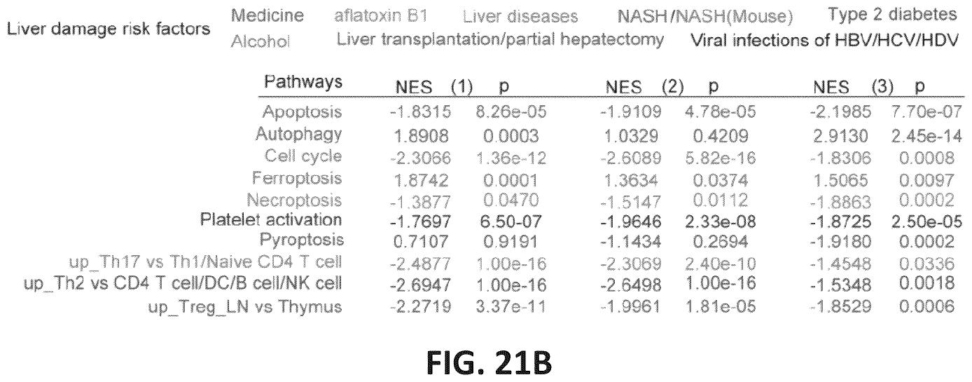

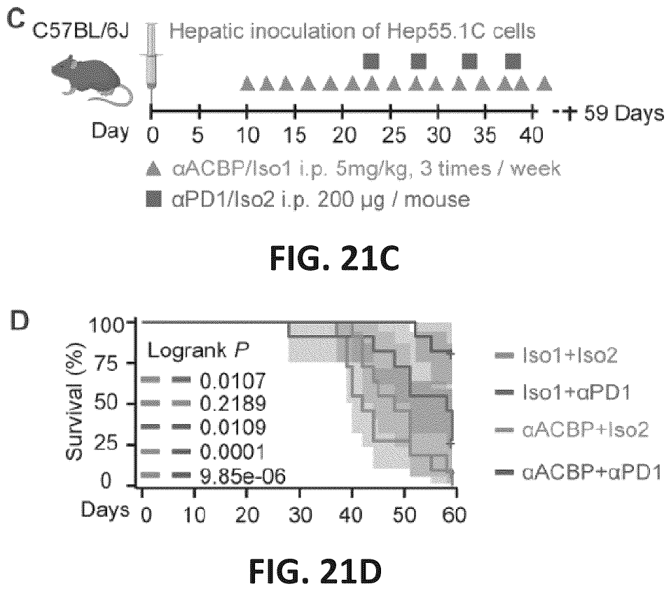

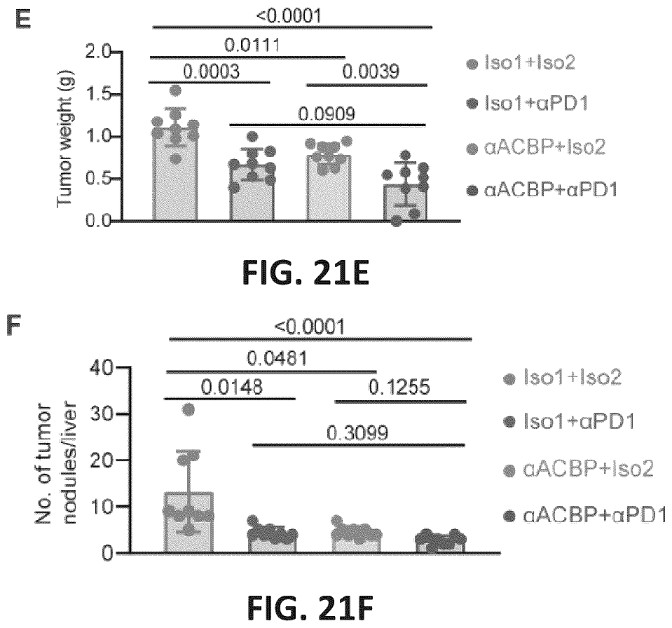

To elucidate the mechanisms through which DBI facilitates hepatic carcinogenesis, liver bulk RNAseq was performed on three of the aforementioned models, namely (i) WD+CCl₄-treated Dbi^{+/+} versus Dbi^{-/-} mice, (ii) WD+CCl₄-treated KLH-only versus KLH-DBI-vaccinated mice, and (iii) HFD+DEN-treated KLH-only versus KLH-DBI-vaccinated animals. The effects of unrestrained DBI action was then compared to 25 publicly available datasets detailing the deregulation of molecular pathways in different liver diseases. In FIG. 21A, normalized enrichment score (NES) of each KEGG pathway was row-normalized and presented as Z-score. Euclidean distance was calculated for row and column clustering analysis. Non-supervised hierarchical clustering indicated that many molecular pathways that are downregulated by DBI inhibition are upregulated in several liver diseases (or conversely molecular pathways upregulated by DBI inhibition are downregulated in such liver diseases), irrespective of their etiology (alcoholic, inflammatory, metabolic or toxic), with the notable exception of infection by hepatitis viruses B, C and D, which do not cluster with DBI inhibition (FIG. 21A).

In the three RNAseq datasets that reflect DBI inhibition, several gene ontology terms suggesting immunosuppression were downregulated, as this applies to a signature specific for regulatory T cells in lymph nodes, pro-inflammatory Th17 cells and polarized Th2 cells (FIG. 21B). According to this prediction, orthotopic Hep55.1C HCC were highly responsive to a combination treatment with a neutralizing mAb against DBI plus a PD-1 blocking antibody. This combination was significantly more efficient in reducing tumor growth than each of the two monotherapies (DBI alone or PD-1 blockade alone), thus markedly extending survival (FIG. 21C-FIG. 21F). Hence, DBI inhibition can enhance the susceptibility of HCC to immunotherapy.

### DBI Neutralization Reduces HCC Proliferation

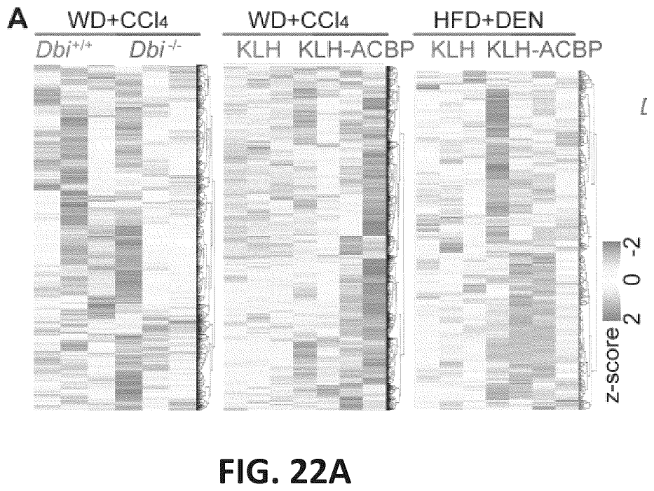

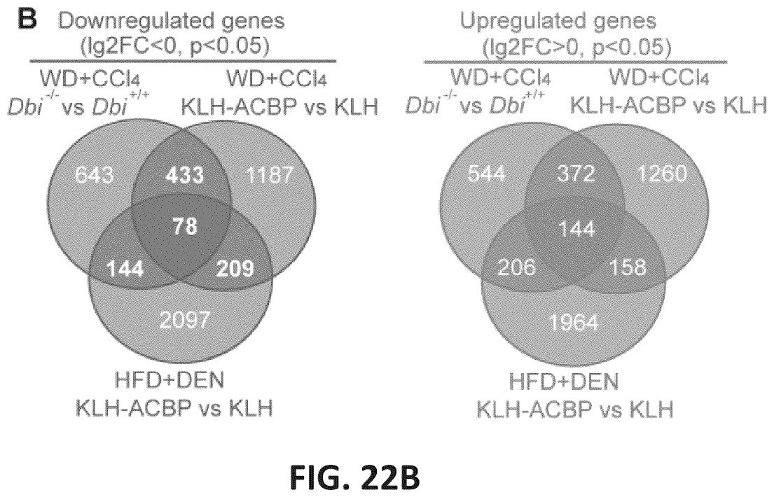

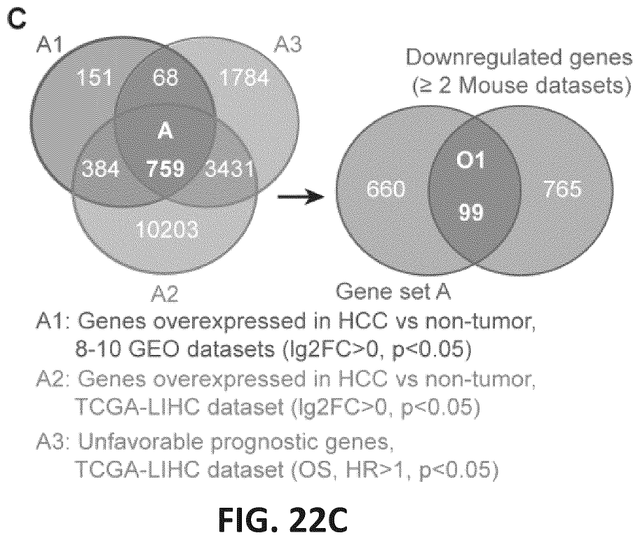

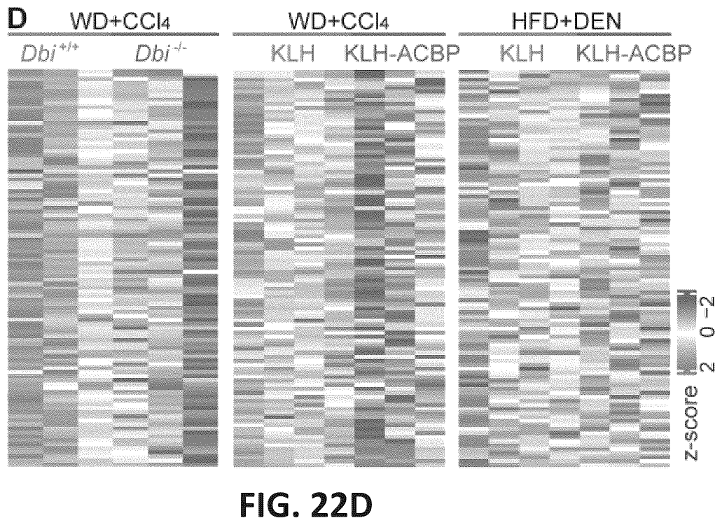

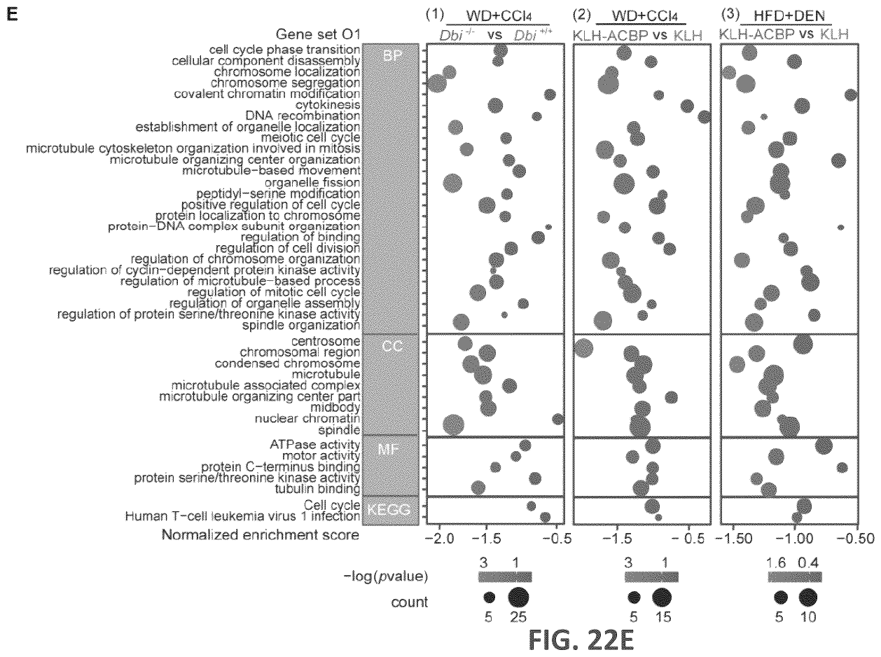

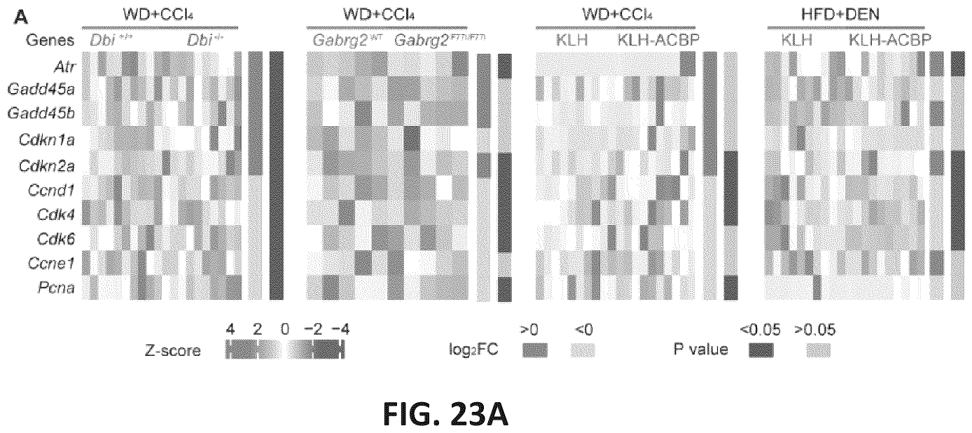

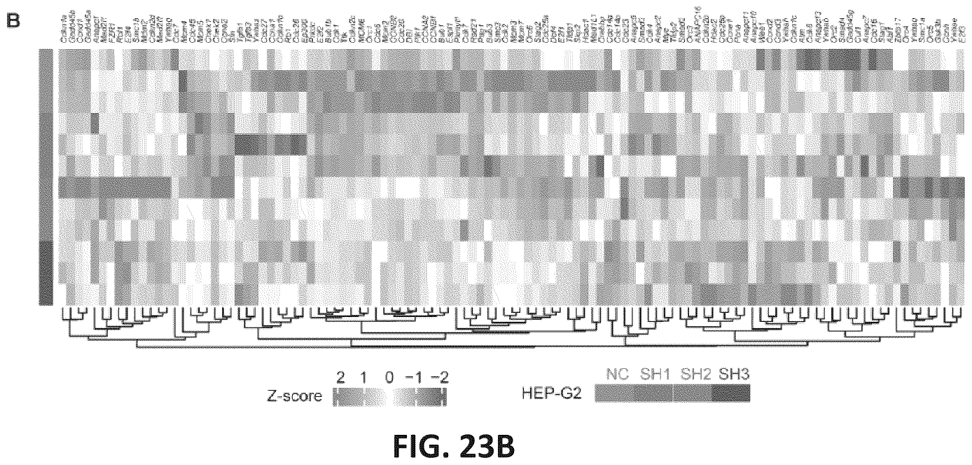

Cell cycle-associated genes were strongly downregulated in all three RNAseq datasets reflecting DBI inhibition (FIG. 21B). A bioinformatic analysis identified commonly up- or downregulated genes in livers from i) WD+CCl₄-treated Dbi^{+/+} versus Dbi^{-/-}, (ii) WD+CCl₄-treated or (iii) WD+DEN-treated KLH-only versus KLH-ACBP/Dbi-vaccinated mice (FIGS. 22A-FIG. 22B). Genes downregulated by DBI inhibition that are also overexpressed in human HCC, as well as associated with poor prognosis were identified (FIG. 22C and FIG. 22D). Among these DBI-dependent, disease-relevant genes, many were associated with cell cycle advancement (and in particular mitosis) (FIG. 22E). qRT-PCR quantitation confirmed that BDI inhibition downregulated genes required for cell cycle advancement (such as Ccnd1, Ccne1, Cdk4, Cdk6 or Pcna) and upregulated genes that block the cell cycle (such as Atr, Gadd45a, Gadd45b, Cdkn1a or Cdkn2a) (FIG. 23A). Similarly, knockdown of DBI in HEP-G2 cell lines elicited transcriptional signs of cell cycle blockade (FIG. 23B), in accord with the reduced oncogenic and proliferative ability of such DBI-depleted cells.

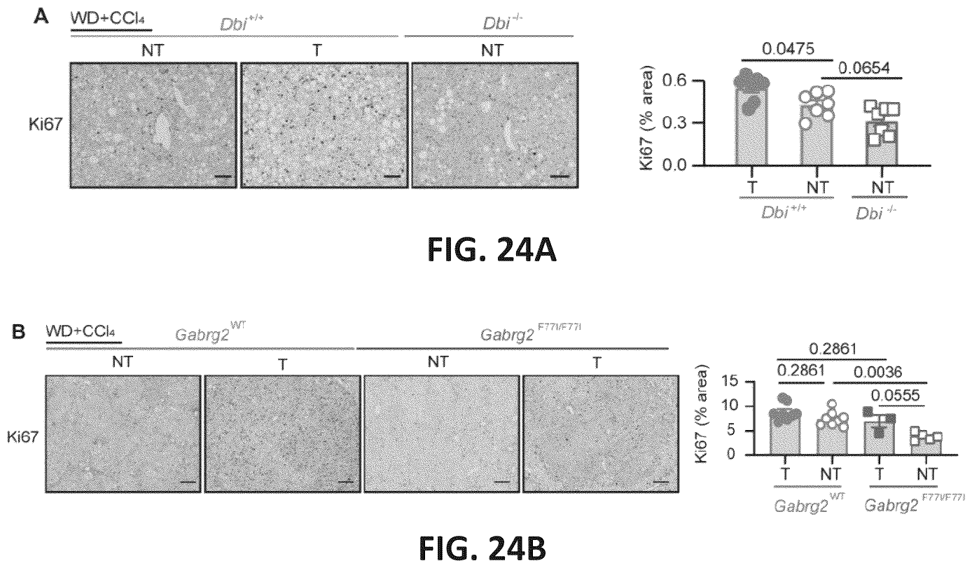

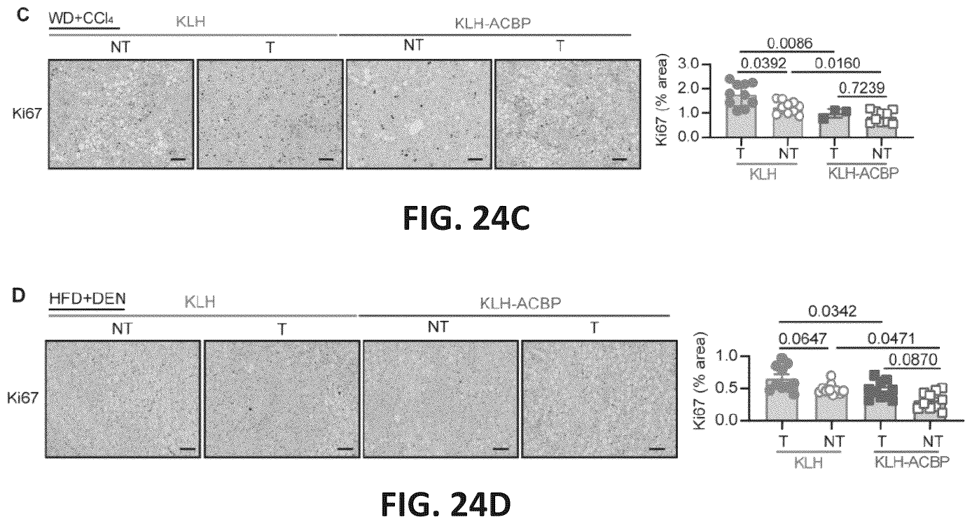

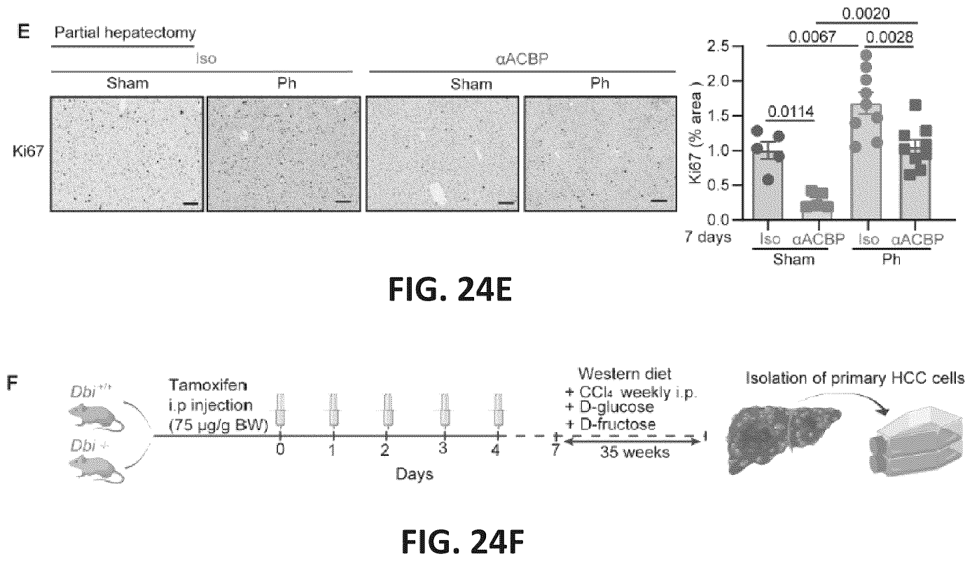

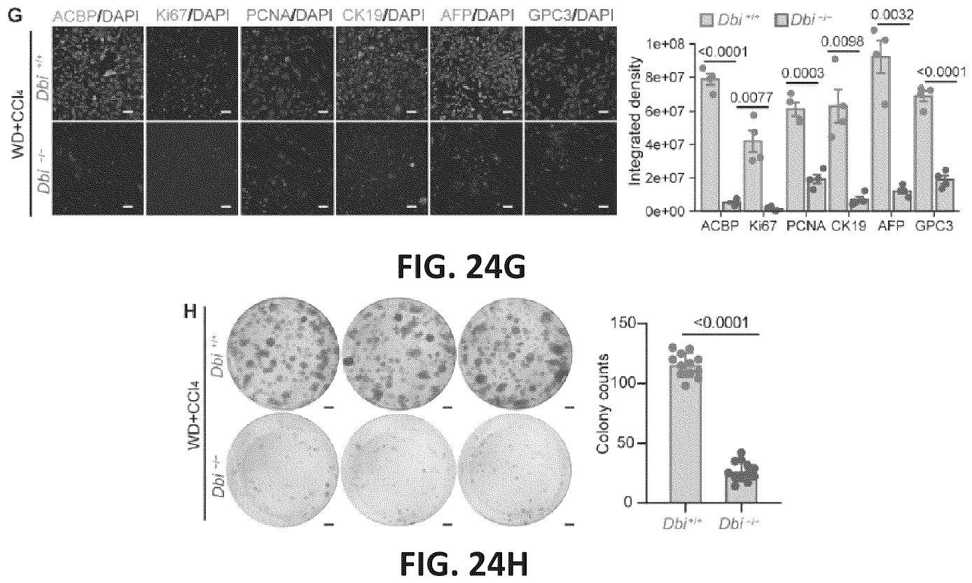

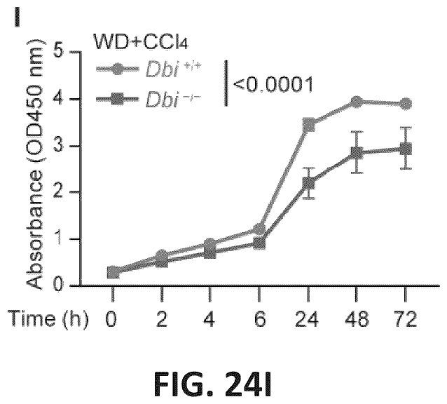

Prompted by these results, cell proliferation in livers from mice subjected to carcinogen-induced oncogenesis was assessed using immunohistochemical detection of Ki67 (FIG. 24A-FIG. 24E) and PCNA. Of note, DBI inhibition by Dbi KO, Gabrg2 mutation or KLH−/Dbi vaccination resulted in reduced proliferation, both in tumor lesions (if detectable) and in the non-malignant hepatic parenchyma, irrespective of the precise oncogenic stimulus (CCl₄ or DEN) (FIG. 24A-FIG. 24E). Neutralization of DBI by means of a monoclonal antibody (mAb) inhibited hepatocyte proliferation both after sham operation and after partial hepatectomy (which induces liver regeneration (FIG. 24E), suggesting a general pro-proliferative effect of DBI in normal hepatocytes.

Finally, HCC cells were isolated from Dbi^{+/+} versus Dbi^{-/-} mice subjected to WD+CCl₄-triggered carcinogenesis (FIG. 24F). Dbi^{-/-} HCC cells exhibited comparatively low expression of proliferation markers (Ki67, PCNA) and malignancy-linked markers (AFP, CK19, GPC3) (FIG. 24G), attenuated clonogenic potential (FIG. 24H) and reduced in vitro proliferation (FIG. 24I). These findings indicate that DBI is required for full-blown HCC proliferation.

Without wishing to be bound by theory, DBI has profuse pro-proliferative effects on normal and transformed hepatocytes.

### DBI Neutralization Sensitizes to the Induction of Ferroptosis.

The aforementioned bioinformatic analysis also identified alterations in a variety of pathways of cellular self-consumption. Thus, DBI suppression entailed a reduction in transcripts relevant to apoptosis and necroptosis, but an increase in the expression of genes relevant to ferroptosis (see FIG. 21B). In addition, DBI inhibition enhanced the expression of autophagy-related genes as well as the pro-ferroptotic effects of autophagy.

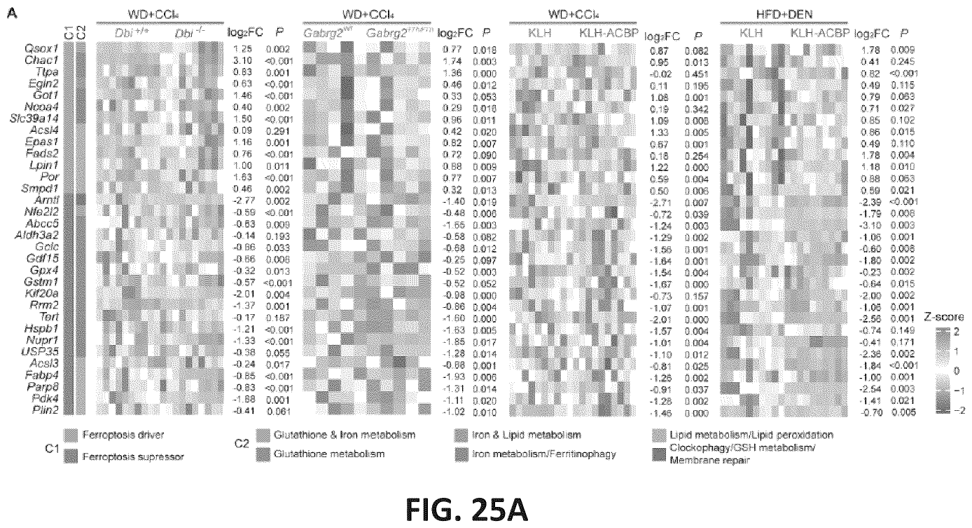

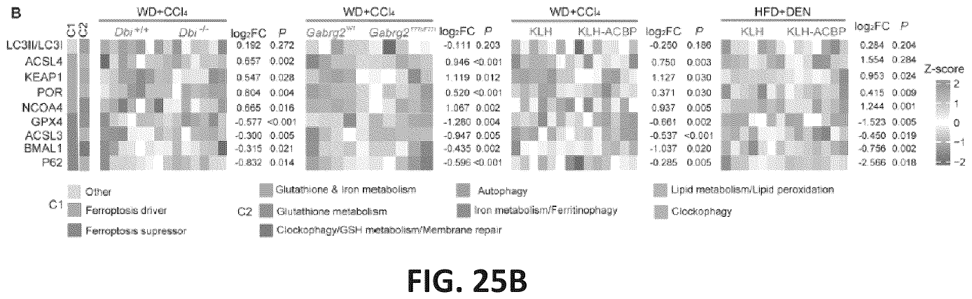

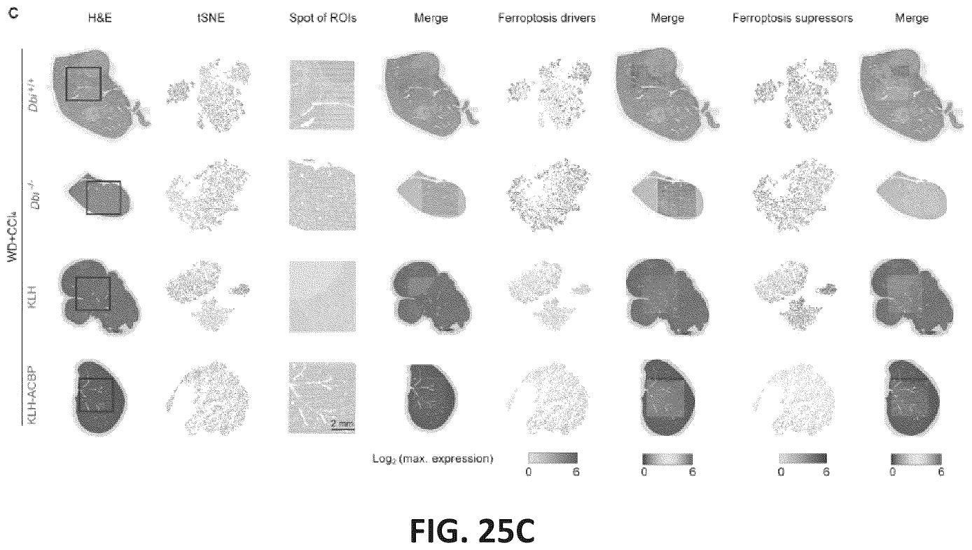

Validation by qRT-PCR confirmed the upregulation of ferroptosis driver genes as well as the downregulation of ferroptosis suppressor genes in livers from i) WD+CCl₄-treated Dbi^{-/-} versus Dbi^{+/+} (ii) WD+CCl₄-treated Gabrg2^{F77I/F77I} versus Gabrg2^{WT} mice, (iii) WD+CCl₄-treated and (iv) WD+DEN-treated KLH-Dbi versus KLH-only-vaccinated mice (FIG. 25A). Moreover, immunoblots confirmed the increased protein expression of ferroptosis effectors (such as ACSL4, KEAP1, NCOA4 and POR) but decreased expression of ferroptosis suppressors (such as ACSL3, BMAL1, GPX4 and SQSTM1/p62) after inhibition of DBI (FIG. 25B). Such shifts in favor from ferroptosis suppression towards execution were also observed for HCC clones derived from the livers of WD+CCl₄-treated Dbi^{-/-} versus Dbi^{+/+} mice, both at the mRNA and at the protein levels. Spatial transcriptomics or spatially resolved mass spectrometric metabolomics coupled to hematoxylin-eosin staining allowed to distinguish non-malignant and malignant areas of the liver (FIG. 25C). Of note, inhibition of DBI by Dbi knockout or KLH-Dbi vaccination revealed a higher expression of ferroptosis effectors in apparently (still) non-malignant tissue compared to DBI-uninhibited controls (FIG. 25C). Hence, the alterations in ferroptosis-relevant gene expression levels found on total livers are detected both in isolated HCC cells, as well as in non-malignant tissues.

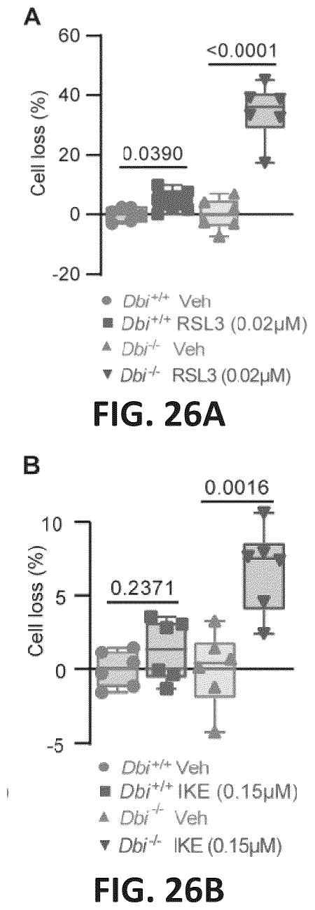

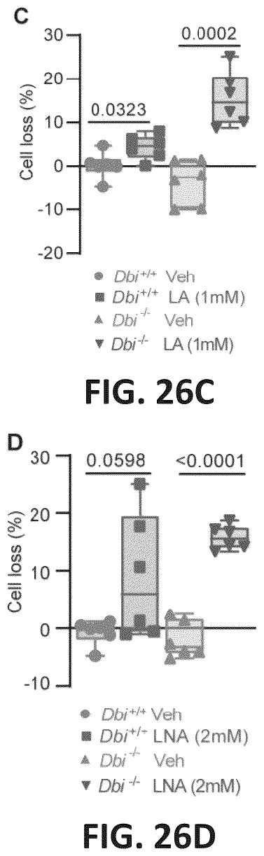

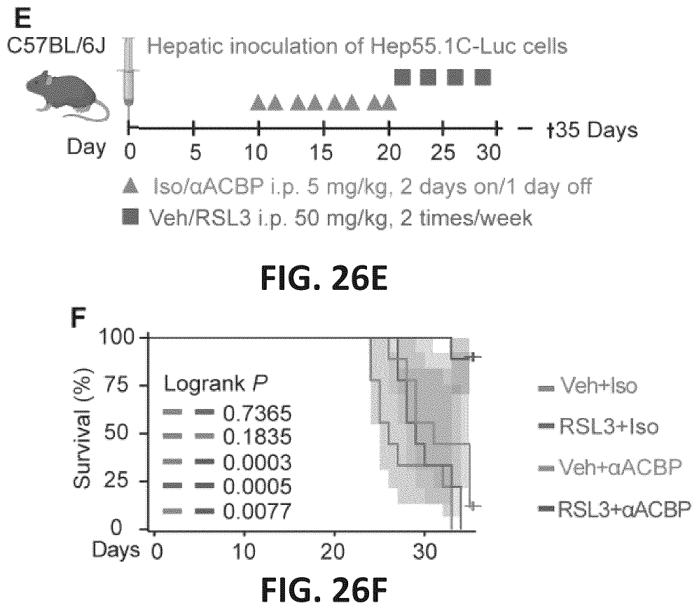

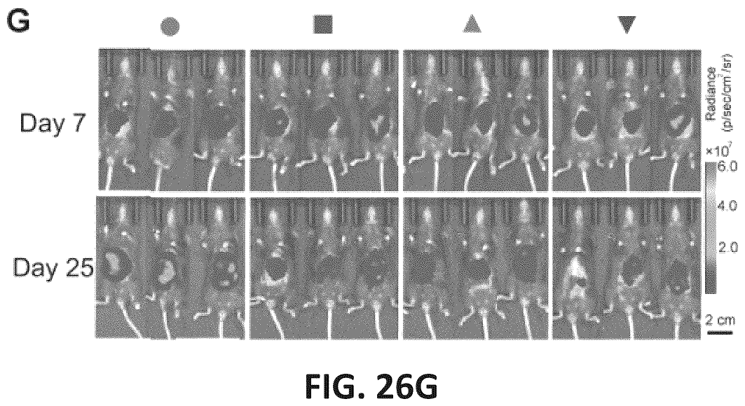

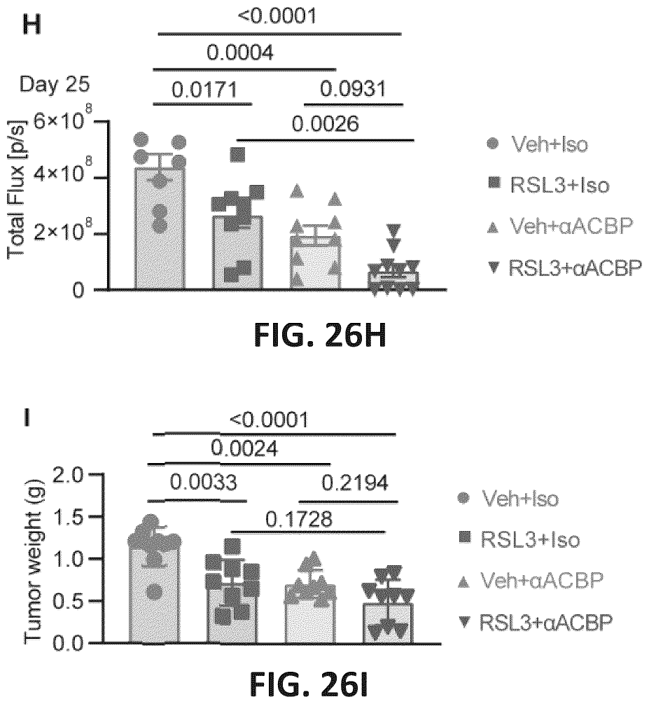

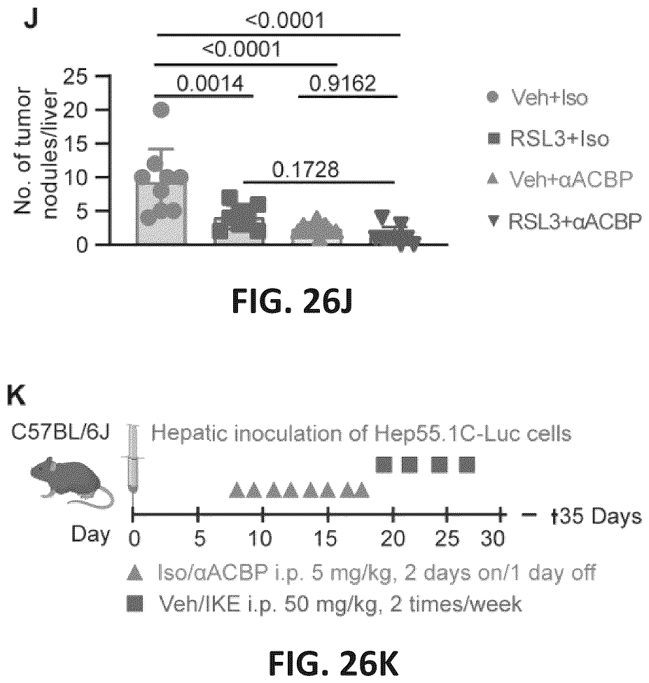

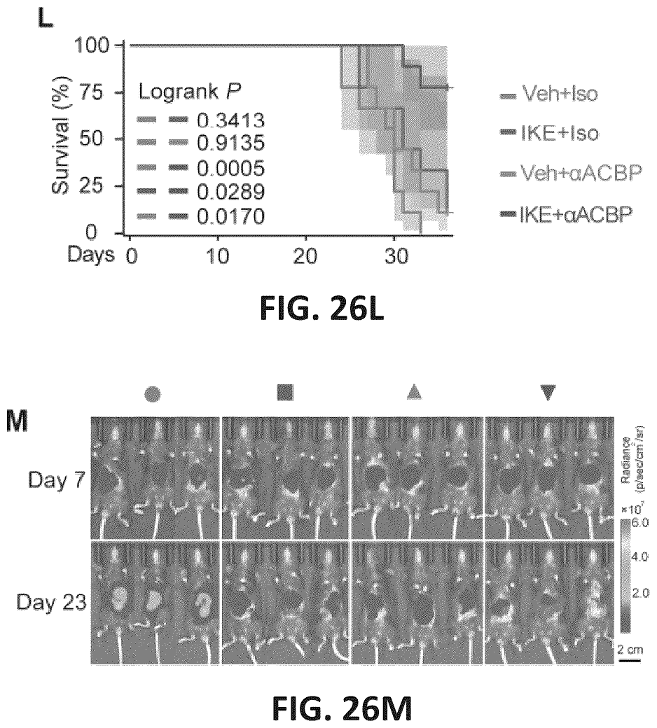

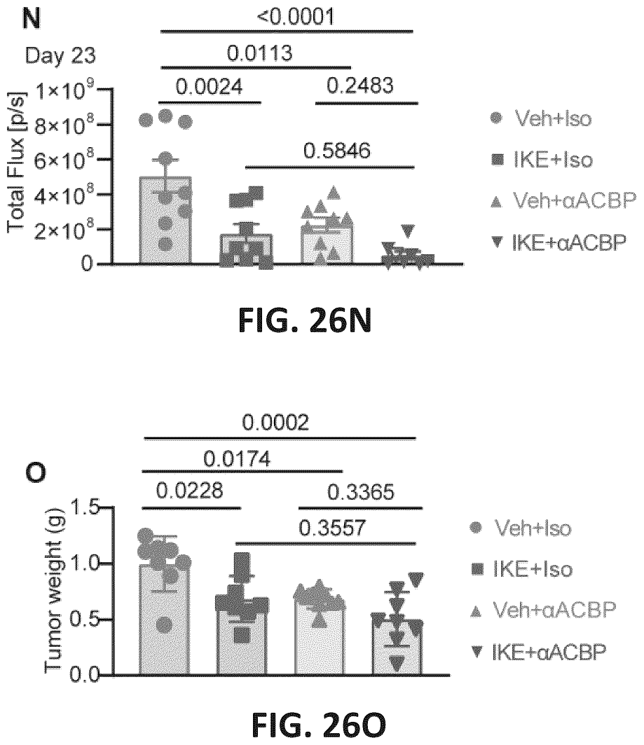

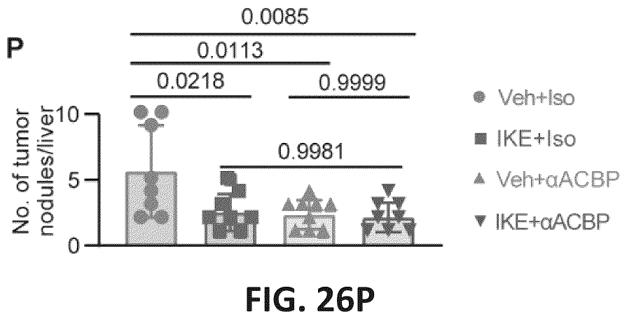

In accord with the gene/protein expression data, HCC cells were particularly vulnerable to losing their viability in response to the combination of the Dbi knockout and the addition of pharmacological ferroptosis inducers including RSL3, IKE (imidazole ketone erastin), LA (linoleic acid), and LNA (linolenic acid). (FIG. 26A-FIG. 26D). Groups receiving ferroptosis inducers were normalized to their respective Dbi^{+/+} and Dbi^{-/-} vehicle (Veh) treated control groups.

Moreover, orthotopic Hep55.1C cancers responded to treatment with a combination of anti-DBI mAb plus ferroptosis induction (with RSL3 or IKE) more efficiently than to standalone therapies with either anti-DBI mAb or ferroptosis inducers (FIG. 26E-FIG. 26P). This combination was more efficient in reducing tumor burden than each of the two monotherapies (anti-DBI alone or RSL3/IKE alone), thus markedly extending survival (FIG. 26E-FIG. 26P).

Without wishing to be bound by theory, inhibition of DBI sensitizes HCC to therapeutic interventions with ferroptosis inducers.

In the present disclosure, Applicants surprisingly found that DBI contributes to the development and progression of HCC induced by a variety of rather distinct methods including (i) intrahepatic inoculation of HCC cells, (ii) oncogene-induced transformation, and (iii) chronic challenge with the hepatotoxin CCl₄ or (iv) the mutagen DEN in the context of Western style diet.

Applicants used a compendium of genetic methods to inhibit the DBI system (i.e., knockdown or knockout of DBI and mutation of its receptor GABRG2), as well as immunological approaches (i.e., neutralizing antibodies against DBI), finding that all these strategies mitigated hepatic carcinogenesis. This preclinical evidence in favor of pro-HCC effects of DBI is backed up by clinical correlations showing that aggressive HCC is associated with upregulation of DBI mRNA, as well as an increase in circulating ACBP/DBI protein concentrations.

Inhibition of DBI consistently reduced signs of liver inflammation leading to excessive macrophage infiltration and fibrosis, but also reduced the expression of genes associated with immunosuppressive T cell subtypes (such a Tregs, T_{H}2 and T_{H}17). Accordingly, inhibition of extracellular DBI by means of a monoclonal antibody sensitized HCC to immunotherapy by PD-1 blockade.

DBI inhibition also resulted in the upregulation of 13 pro-ferroptotic mRNAs and the downregulation of even more anti-ferroptotic mRNAs. This effect, which could be validated at the protein level, was accompanied by increased susceptibility HCC cells to pharmacological induction of ferroptosis both in vitro and in vivo.

Without wishing to be bound by theory, neutralization of DBI reduces or delays the manifestation HCC in various preclinical models. In addition, inhibition of DBI inhibits proliferation and sensitizes established HCC to PD-1-targeted immunotherapy, as well as pharmacological induction of ferroptosis.

### Materials and Methods

### HCC Patient Cohort

A total of 260 plasma samples were collected from 146 patients with HCC and 58 patients with chronic liver diseases without HCC at the Avicenne tertiary University Hospital (Bobigny, France) between March 2013 and May 2021. Two or more plasma samples were collected from 46 patients. Plasma samples were divided into two groups; (i) plasma collected at the time of diagnosis of HCC, the day of treatment or at the time of radiological evaluation showing active HCC (n=195), (ii) plasma collected in patients with chronic liver disease without HCC or plasma collected after HCC treatment without any active tumor at imaging (n=65). Whole blood (5 mL) was collected using EDTA tubes. Blood samples were centrifuged at 2000×g for 10 minutes at room temperature and were immediately stored at −80° C. All patients signed an informed consent for sample collection and the ethics committee approved the study.

Patient, tumor characteristics, and treatment types were obtained from medical records. Patient baseline characteristics were collected before treatment; including age, gender, etiology of liver disease, and presence of cirrhosis. Cirrhosis was defined either by histology or by the combination of clinical-biological data, ultrasonography, and liver stiffness measurement. HCC was diagnosed at histology or using non-invasive criteria through imaging techniques (magnetic resonance imaging [MRI] and/or triphasic computed tomography [CT]) according to the European Association for the Study of the Liver (EASL) guidelines. Tumor characteristics at imaging (tumor size and number, macrovascular invasion, and metastasis), Barcelona Clinic liver cancer staging system (BCLC), alpha-fetoprotein (AFP), and Child-Pugh (CP) score were also collected. Data about the type of treatment (ablation, embolization, and/or systemic treatment), and radiological response assessed by mRECIST criteria at 4 and 12 weeks after locoregional and systemic treatment respectively, were also recorded.

### Generation of Stable Cell Lines, Cell Culture, and Cell Assays

Generation of stable DBI knockdown liver cancer cell lines. Liver cancer cells were grown under the following conditions; HEP-G2 (EMEM+10% FBS+1% sodium pyruvate+1% HEPES), HUH-7 (DMEM, 10% FBS), Hep55.1C (DMEM, 10% FBS) and Hep55.1C cells expressing firefly luciferase (Hep55.1C-luc, DMEM, 10% FBS, 0.1% blasticidin). These cancer cells were grown in 6-well dishes to 60-70% confluency then transfected using 25-35 μL lentiviral shRNAs targeting DBI (SH1, SH2, SH3) or negative control (NC) with 5 μg/mL polybrene (5-8 μL) mixed thoroughly in 1 mL medium. The medium was replaced with fresh medium after 24-48 h, and the cells were maintained for another 24 h. Puromycin (10 μg/mL) was used to select the transduced cells. Single cell clones were isolated by single-cell FACS sorting in 96-well plates and gradually expanded in 24-well plates, 12-well plates and 6-well plates. All the cell clones from 6-well plates were duplicated, one stocked at −80° C. for stock and one collected for qRT-PCR to detect the knockdown efficiency of DBI. Clones with the highest knockdown efficiency were chosen to perform further assays.

Proliferation assays. CCK-8 assays were performed with a commercial kit. For colony formation assays, HEPG-2 (NC, SH1, SH2, and SH3, 1000 cells/well), HUH-7 (NC, SH1, SH2, and SH3, 1000 cells/well) and Hep55.1C (NC, SH1, SH2, and SH3, 500 cells/well) cancer cells were seeded into 6-well plates in 2 mL medium with 10% FBS. The medium was changed every 3 days until control (NC) wells approached confluency, within 7-14 days. After gently washed twice with PBS, colonies were fixed with 4% paraformaldehyde for 30 min and stained with 0.2% crystal violet for 30 min at room temperature. The colony number (colonies >0.3 mm) was counted with isoftware. All experiments were performed in triplicates.

Cell cycle analysis. Following the instructions of a commercial kit, the aforementioned transfected cells (25×10⁴ cells/well) were plated in 6-well plates for 24 hours. Cell cycle was then synchronized with medium containing 0.1% FBS for 24 h, followed by culture in medium with 10% FBS for 24 h. The cells were harvested and fixed using ice cold 70% ethanol. The cell cycle distribution was then determined by flow cytometry.

### Animals

All mice used in this study had a C57BL/6 background. Five mice/cage were housed in a 12 h light/dark cycle under specific pathogen-free conditions. Mice were allowed at least 1 week of acclimation to the new housing facilities prior to use. All mouse experiments were performed according to protocols approved by the local Animal Experimental Ethics Committee. Liver tissues and plasma samples were collected for further analysis. Liver tissues were freshly frozen in liquid nitrogen and stored at −80° C., fixed in 4% paraformaldehyde, or embedded in optimum cutting temperature compound (OCT).

### Inhibition of DBI In Vivo

Four strategies were developed to inhibit the expression or function of DBI. (i) Tamoxifen-inducible ACBP DBI knockout. Mice with tamoxifen-inducible ACBP/DBI knockout were generated by crossing ACBP/DBI^{fl/fl} mice with B6.Cg-Tg(UBC-Cre/ERT2)1Ejb/1J mice, followed by tamoxifen injection (i.p. 75 mg/kg/day, for 5 consecutive days). Control mice were ACBP/DBI^{fl/fl} mice without Cre. Tamoxifen was dissolved in corn oil (90%)+ethanol (10%) at a concentration of 20 mg/mL, aliquoted and stored at −20° C. (ii) Constitutive Gabrg2^{F77I/F77I} point mutation. Gabrg2^{tm1Wul}/J mice containing the point mutation F77I were compared to control mice with wild type mice without the mutation. (iii) Induction of ACBP/DBI-specific autoantibodies. As previously described, KLH was conjugated with recombinant DBI at 1:30 molar ratio to produce KLH-DBI. KLH alone was used as control. Montanide (and KLH/KLH-DBI were then mixed a 1:1 volume ratio. The mice were i.p. vaccinated with the aforementioned mixture on days 0 (30 μg), 7 (30 μg), 14 (30 μg), and 21 (10 μg of KLH-DBI) to induce anti-DBI autoantibodies. Immunoblots of recombinant DBI were incubated with plasma from KLH/KLH-DBI-immunized mice to detect anti-ACDBIBP antibody. (iv) Monoclonal anti-DBI antibody. Mice were i.p. injected with anti-DBI mAb (a-DBI, 5 mg/kg) or its control isotype (5 mg/kg) 3-4 times/week to neutralize DBI.

### NASH-Driven HCC Model

Three major types of HCC mouse models were tested in this study. (i) Diet plus toxin-induced mouse models of were based on a western diet (WD) or high-fat diet (HFD) plus carbon tetrachloride (CCl₄) or diethylnitrosamin (DEN), respectively. In most experiments, mice received a western diet (WD, i.e., a high-fat, high-fructose and high-cholesterol diet), high-sugar water (23.1 g/L D-fructose plus 18.9 g/L D-glucose), and weekly i.p. injections of carbon tetrachloride (CCl₄, 1:10 diluted in corn oil) at a final dose of 2 μL/g of body weight. High-sugar water was added in the sterile 450 mL sipper sack bags and replaced weekly. Also, food was added weekly. Male mice were used in this HCC model. Three DBI inhibition strategy (DBI knockout, Gabrg2^{F77I/F77I} mutation, and KLH-ACBP vaccination) were combined with this mice model. Mice were sacrificed after 27-33 weeks, based on hepatic tumor detection by means of medical ultrasound (also called sonography). At necropsy, tumor number and size were determined by counting the number of visible tumors and measuring their size with a caliper. Body weights of mice were monitored weekly. HFD-DEN induced HCC mouse model (combined with KLH-DBI) was used to confirm data from WD plus CCl₄ mouse model. For this, male C57BL/6 mice (15 dpp) were treated with a single dose of diethylnitrosamin (DEN, N0258) dissolved in saline at a dose of 25 mg/kg body weight by intraperitoneal injection. After 2 weeks, concomitant with the weaning, mice started the vaccination protocol and HFD composed by 60% fat calories. Fresh diet was provided every 2 to 3 days and body weights were recorded monthly. Mice were sacrificed after 36 weeks and livers explanted to measure tumor number and size. Vaccination was performed with KLH/KLH-DBI. Specifically, for the vaccination protocol, DEN injected mice (28 dpp) were randomized in two groups.

### Oncogene-Induced HCC

Hydrodynamic transfection of oncogenes (Myc+Ctnnb1). A mixture of transposase-encoding vector (SB100, 1.5 μg), pT3-EF1a-Myc plasmid (7.5 μg), and pT3-N90-Ctnnb1 plasmid (7.5 μg) were prepared fresh in 1 mL 1×PBS at a ratio 1:5:5. The final solutions was sterile-filtrated through a 0.22 μm filter and placed in a 37° C. water bath prior to use. A volume equivalent to 10% of mouse body weight (1 mL/10 g) was injected via tail veins with high pressure within 8-10 s, using a 3 mL syringe with 30 G×½ needles in female mice (7 weeks-old). Combinations of the aforementioned four DBI inhibition strategies and this HCC mouse models were tested to and tumor formation were monitored with ultrasound. The survival was monitored and mice were sacrificed at the humane endpoint, defined by mouse grimace scale, notable abdominal distension caused by tumor burden, >20% initial body weight loss, or largest tumor size reaching 20 mm based on ultrasound. The tumor number and size were measured and recorded at necropsy.

### Orthotopic Transplantation of HCC Cells

Murine HCC cell lines (Hep55.1C and Hep55.1C-derived cell lines such as Hep55.1C-Luc, or Hep55.1C/Hep55.1C-Luc-NC, -SH1, -SH2, and -SH3, 30×10⁴/50 μL/mouse in DMEM without FBS) and human HCC cells (HUH-7, 200×10⁴/50 μL/mouse in DMEM without FBS) were transplanted into mouse liver to generate orthotopic liver tumors in female C57BL/6J mice or male nude mice, respectively. Tumor growth of mouse models implanted with luciferase-labeled Hep55.1C-Luc was monitored by IVIS bioluminescence in vivo imaging system, otherwise via ultrasound unless noted. Mice were monitored for survival and symptoms (such as skin jaundice). Mice were sacrificed at the humane endpoints mentioned above.

### Partial Hepatectomy

9 weeks-old male mice were submitted to a 70% partial hepatectomy (Ph). Briefly, mice were administered with buprenorphine for analgesia and anaesthetized by inhalation of isoflurane (2%). Then, a midline abdominal skin and muscle incision was made to expose the peritoneal cavity, followed by the removal of the left and median hepatic lobes. First, a 4-0 silk thread was placed on the base of the left lateral lobe, the knot was tied and the tied lobe was cut just above the suture. The same procedure was done with the median lobe. Finally, the peritoneum was closed with a 5-0 suture and the skin with wound clips. Sham controls mice were submitted to the same surgical procedure without removing the liver lobes. Mice were randomized into four groups (Sham+Isotype, Sham+anti-ACBP, Ph+Isotype and Ph+anti-ACBP), 9-10 mice/group. Anti-Acbp mAb (i.p. 2.5 mg/kg) or Isotype (i.p. 2.5 mg/kg) were administered 4 h and 1 h before the surgery and three more times before sacrifice. Seven days after the partial hepatectomy, animals were sacrificed, and the remaining liver was harvested and processed for further analysis.

### Isolation of Primary HCC Cells

Six-well plates were coated with 2% FBS/PBS+0.1% laminin. HCC tumors were isolated from NASH-driven mouse model (WD+D-fructose+D-glucose in water+CCl₄ administered to DBI^{+/+} or DBI^{-/-} mice) and washed twice with ice-cold PBS. The tumor tissues were transferred to 10 cm plates, minced completely with a razor blade and then transferred to 15 mL falcon tubes. Ten mL 2 mg/mL collagenase dispase solution was added, the cells were incubated rotating for 30 min at 37° C. and were filtered through 100 μm meshes into 50 mL falcon tubes, before addition of 10 mL 2% FBS/PBS to wash the tissues. The samples were then filtered sequentially through 70 μm and 40 μm strainers, brought up to 15 mL volume and centrifuged again (room temperature (RT), 1000 rpm, 2 min). The cells were then resuspended in 5 mL of 1×red blood cell (RBC) lysis buffer, incubated on ice for 10 min before addition of 5 mL 2% FBS/PBS, followed by spinning (RT, 1000 rpm, 2 min). The washing step was repeated 2 times, and the cells were resuspended in culture medium (500 mL DMEM+10% FBS+1×ITS+20 μl EGF (1 μg/μl)+20 μl IGFII (0.2 μg/μl)), before seeding into 6-well plates.

### Multi-Omics Analysis

Bulk RNA-sequencing. Murine liver tissue samples were homogenized in QIAzol lysis reagent. Total RNA was extracted and purified by means of the miRNeasy Mini Kit. Total RNA was subjected to RNA sequencing on sequencing instrument. The RNA sequencing data were obtained in Fasta file format. Reads were mapped to mouse genome assembly (GRCm39, mm10) using HISAT2, followed by read counting with HTSeq-count. The differential expression analysis was estimated using DESeq2 R-package. Variance stabilizing transformation (VST) was used to transform and normalize the count matrix as implemented in DESeq2. Gene set enrichment analysis (GSEA)-based KEGG pathway and gene ontology (GO), including biological process (BP), molecular function (MF), and cellular component (CC), were performed with the differential expression data a web-based tool for functional enrichment analysis. Software packages were used to visualize data.

Spatial transcriptomic. Tissue samples were initially screened for RNA quality, ensuring DV200 scores exceeded 50%. using spatial gene expression slides and reagent kits. Five-micrometer thick tissue sections were cut from the FFPE tissue blocks and mounted on slides. The sections underwent initial deparaffinization, imaging and permeabilization steps before being meticulously hybridized with the probes. Then, probes pairs that had successfully hybridized to RNA were ligated to facilitate the sealing of junctions between them and finally released from the tissue within the instrument for capture on the FFPE Visium Spatial Gene Expression Slide within the fiducial frame.

Each capture area comprised approximately 5000 gene expression spots, that include an sequencing primer, 16 nucleotide (nt) Spatial Barcode, 12 nt unique molecular identifier (UMI), 30 nt poly(dT) sequence (SEQ ID NO; 3) (captures ligation product). These spots offered a resolution of approximately 5-10 cells. Ligation products were extended by the addition of UMI, partial Read 1, and spatial barcodes, ultimately yielding spatially barcoded products for subsequent library preparation. qRT-PCR was meticulously employed to ascertain cycle numbers, and the ligated and spatially barcoded products underwent indexing via Sample Index PCR. The sequencing of libraries was executed on a sequencing instrument with a SP flow cell (100 cyclesFASTQ files processed to generate the .cloup files. tSNE and spatial plots were run and plotted using Loupe Browser (10× genomics).

Spatial metabolomics. The details including sample prepare, H&E staining, mass spectrometry imaging (MSI) acquisition, and data analysis. (i) Sample preparation for MSI. Liver tissues were harvested from mice, rinced with PBS and placed in embedding cassette with one piece of foam pads then gently frozen in steam bath of liquid nitrogen. Cassettes were stored at −80° C. Tissue sections were performed with a cryotome HM 500 O (Micro). Tissues were warmed from −80° C. to −15° C. in 20 min. The piece of tissue was mounted with milliQ water on a peltier platform, and the object was fast-frozen for 10) minutes. Sections of tissues were cut with a thickness of 20 μm and put on labelled microscope slides. Three slides were alternatively dedicated to the Mass Spectrometry Imaging (MSI) positive mode, MSI negative mode and Hematoxylin and Eosin (H&E) staining. MSI Mass spectrometry imaging (MSI) sections were frozen in slide mailers at −80° C. and H&E staining was performed right after cutting. (ii) H&E staining. Sections on the microscope slide were successively bathed in 100%, 70% and 50% of ethanol (EtOH), each step during 2 min, then followed by 2 min in tap water. Tissues were afterward colored with hematoxylin for four 4.5 min, and rinsed with water for 4 min. Second coloration was done with eosin for 2.2 min, rinsed again with water for 1.5 min. Three last baths were applied on the sections, respectively at 50, 70 and 100% of EtOH, for 1 min of each. Finally, a quick rinse with Xylene was done before mounting cover slips with Entellan. (iii) MSI acquisition. Full scan (50 m/z to 1200 m/z) acquisitions were performed in sensitive mode with a synapt XS waters equipped with a DESI XS source. Three slices were acquired in positive polarity acquisition mode, and three others were used for negative polarity acquisition mode. Tissue sections acquired in negative polarity were sprayed with 2 μl/min of methanol/water buffer (96/4, with 1 mmol of ammonium acetate) with lockspray leucine-enkephaline (250 ng/ml). Capillary voltage was set at 0.70V, sampling cone at 40V, source temperature at 150° C. Regarding the negative polarity acquisition mode, the 2 μl/min of buffer was composed of methanol/water (82/18) with 0.1% of formic acid and 250 ng/ml of leucine-enkephaline. Capillary voltage was set at 0.40V, sampling cone at 40V, source temperature at 150° C. DESI source was scanning the tissue spatial resolution with a spatial resolution of 50 μm² per pixel, and a scan time of 0.153 s. Acquisition parameters were set with HDI v1.6 and acquired with MassLynx v4.2. (iv) Data analysis. Raw data were processed by HDI v1.6. A target list was built from one replicate, based on the thousand most intense ion signals. Then we added to this data-driven built list our own targeted list of special interest. It is resulting to an identical list of m/z searched throughout pixels (corresponding to mass spectra) of the whole set of MSI files. HDI raw outputs were exported in text files and import in R software to further data treatments. Data handling with R included raw data cleaning (image cropping, mis-acquired replicate exclusion) and normalization by total ion count (TIC). A smooth function was applied on the MSI files, in order to limit the artefactual signal variations between pixels. Then, artefactual metabolites were removed; if the mean of the tissue region of interest (tissue ROI, based on Kmeans pixels clutering) was less than the mean of the glass ROI for more than the half of the MSI files, then metabolites were excluded from ulterior analysis. All pixel values corresponding to the tissue replicates were then gathered and analyzed by a centered and non-scaled principal component analysis (PCA). PCA values were then clusterized by a Kmeans method, and each pixel was assigned by a color code depending on its cluster. Images of the replicated tissue sections were then reconstructed from this color code assignation, with common ROI to the whole set of files. Finally, each ions signal was meaned by ROI and value were computed in a heatmap.

### Combination Therapies

Anti-DBI plus anti-PD-1. Hep55.1C cells (3×10⁵/50 μL/mouse in DMEM without FBS) were inoculated into the liver of 7-week-old female C57BL/6J mice. Mice were randomized into four groups (isotype1+isotype2, isotype1+anti-PD1, anti-ACBP+isotype2, and anti-ACBP+anti-PD1), 9-10 mice/group, 10 days after tumor initiation. Treatment with anti-DBI mAb (i.p. 5 mg/kg) or isotype1 (i.p. 5 mg/kg) was then started and administered 3 times/week. Treatment of anti-PD1/isotype2 (i.p. 200 μg/mice) started at the week after 6-time dosing of anti-ACBP/isotype1. Mice were sacrificed at the humane endpoint mentioned above. In FIG. 21 and FIG. 26, Iso represent isotye, and DBI/αPD1 indicate anti-DBI/anti-PD1, respectively.

Anti-DBI plus ferroptosis inducers. Hep55.1C-Luc cells (3×10⁵/50 μL/mouse in DMEM without FBS) were inoculated into the liver of 7-week old female C57BL/6J mice. The tumors were assessed by in vivo bioluminescence imaging using the IVIS system (i.p. injection of 200 μL 15 mg/mL luciferin/mice 5 min before detection) at day 7 after tumor initiation. The mice were divided into 4 groups (isotype+vehicle, isotype+RSL3/IKE, anti-DBI+vehicle, anti-DBI+RSL3/IKE) based on the bioluminescence imaging. Treatment with anti-DBI/isotype (i.p. 5 mg/kg) was then started with a regular dosing schedule, 2 days on/1 day off for 8 out of 10 days. RSL3 and IKE were administered at 50 mg/kg for 4 times in 2 continuous weeks, started at day 21/19 from tumor inoculation. Tumor growth was monitored for 4 weeks. Mice were sacrificed at endpoints mentioned above.

### Liver Histology and Immunohistochemistry

Liver tissues in cassettes were fixed with 4% paraformaldehyde for 24 h, and transferred into 70% ethanol. After dehydration steps, cassettes were embedded in paraffin. The liver sections were stained with hematoxylin and eosin (H&E) and Sirius red for histology assessment. Slides were scanned and imaged using a slide scanner. NAFLD Activity Score (NAS) was examined by an expert pathologist, who was blinded to the treatments received, according to the NASH-CRN scoring system. Fibrosis stage was evaluated by Sirius red staining and quantified as % Sirius red+ area by QuPath software. The proliferation score was evaluated by Ki67 and PCNA IHC staining of the liver sections. The image analysis was performed with QuPath software as % positive area.

### Immunofluorescence

Primary HCC cells (DBI^{+/+} and DBI^{-/-}) were seeded at 2000 cells/well using four replicates. After 24 h, the cells were fixed with 4% PFA/PBS (100 μL/well) for 20 min at room temperature (RT). Fixed plates were washed twice with PBS (200 μL/well), permeabilized and blocked with 0.1% Triton X-100+5% BSA+10% FBS for 60 min (100 μL/well) at RT. Primary antibodies were diluted to the appropriate concentration in 1% BSA (100 μL/well), and incubated overnight at 4° C. After two steps of PBS washing (200 μL/well), secondary antibodies (1:250) and DAPI (1:5000) in 1% BSA (50 μL/well) were subsequently added to the cells and incubated for 1 h at RT in the dark. Plates were washed twice with PBS followed by adding PBS (100 μL/well). Image acquisition and analysis were performed with software.

### Enzyme-Linked Immunosorbent Assay

For enzyme-linked immunosorbent assays (ELISA), whole blood was collected from mice. Plasma was isolated by centrifugation at 5000×rpm, 4° C. for 10 min. DBI levels in plasma were assayed by ELISA. High binding ELISA plates were coated with 100 μL anti-DBI antibodies (human: MBS768488, mouse: ab231910) overnight at 4° C. Plates were washed twice with 100 μL washing buffer (PBS+0.05% Tween 20) and blocked with 200 μL blocking buffer (PBS+1% BSA+0.05% Tween 20) for 2 h at RT. After washing, 100 μL standards or diluted plasma samples (human: 1/50, mouse: 1/20) were added to the plates and incubated for 2 h at RT. Plates were washed 3 times with washing buffer, followed by incubation with 1 μg/μl anti-DBI detection antibody (100 μL/well, human; LS-C299614, mouse; MBS2005521) for 1 h at RT. After 3 times washing buffer, 100 μl of diluted Avidin-HRP (human: 1/5000, mouse: 1/1000) was added to each well and incubated at RT for 30 min. The plates were washed 3 times and 100 μl substrates were added, followed by 30-minute incubation in dark. After which 50 μl stop solution was added and the absorbance at 450 nm was read by a microplate reader.

### Quantitative Real-Time PCR

Liver tissues (about 25 mg) were homogenized in a lysis reagent. Total RNA was extracted and purified. Total RNA (1 000 ng) was reversed transcribed into cDNA. Quantitative real-time PCR (qRT-PCR) detection was carried out

### Immunoblotting

Liver tissues were homogenized in protein lysis buffer (20 mM Tris buffer pH 7.4+150 mM NaCl+1% Triton X-100+10 mM EDTA+ protease inhibitor cocktail) by an Homogenizer. Protein extracts were centrifuged at 12000 g (4° C.) for 15 min to collect supernatants. Protein concentration was determined by BCA protein assay kit and samples were then boiled in Laemmli buffer for 10 min at 100° C. Total protein (25 μg) of each sample was loaded onto the 4-12% NuPAGE Bis-Tris gel and transferred to PDVF membranes. To simultaneously detect different antigens within the same experiment, the membranes were sliced horizontally in several parts based on the molecular weight of the protein of interest. Membranes were blocked with 5% (w/v) BSA in TBST (TBS with 0.1% Tween-20) for 1 h at RT and incubated with the appropriate antibodies in 5% (w/v) BSA in TBST overnight at 4° C. Detection was performed with incubation of appropriate horseradish peroxidase (HRP)-conjugated secondary antibodies for 1 h at RT. Blots were visualized by ECL detection reagent. Densitometric analysis were performed using software. GAPDH serves as a loading control. Densitometric ratios of target protein/GAPDH were normalized to control groups, respectively.

### Bioinformatics Analyses

For DBI differential expression of cancer patients, RNA-seq data were downloaded from a TCGA database, extracted in TPM format and presented as log 2 (value+1). DBI differential expression (presented as log 2 (value)) in HCC patients were further validated by analyzing data from HCC-related GEO datasets. For HCC patient plasma analysis, continuous variables are presented as median and interquartile range, and categorical variables as number and percentages. Correlations between continuous variables were assessed using Spearman's rank correlation analysis. Survival curves plotted using Kaplan-Meier (KM) analysis was undertaken. Liver diseases related GEO datasets were downloaded from the GEO database through the GEOquery package. The data were normalized. Only the probe with the largest signal value was retained when encountering multiple probe values for the same gene. GSEA-based KEGG pathway analysis was performed using an online tool for functional enrichment analysis in various biological contexts. The similarity of the global transcriptome dysregulation of the aforementioned liver disease GEO datasets and three NASH-driven mouse RNA-seq datasets in this study (WD+CCl₄_ACBP/DBI knockout, WD+CCl₄_KLH-ACBP, and HFD+DEN_KLH-ACBP) were analyzed by row and column clustering analysis (Euclidean distance) of the normalized enriched scores (NES). Statistical analyses and data visualization were performed.

### Statistical Analysis

Unless indicated otherwise, data are presented as means±SEM. Normality was assessed by normality tests. Normally distributed data were analyzed by T test, one-way ANOVA or two-way ANOVA. Non-normally distributed data were analyzed using the Welch's t test (two groups), Mann-Whitney U test (two groups) or Kruskal-Wallis test (multiple groups) followed by Dunn's post-test. Body weight curves were analyzed using two-way ANOVA performed through the Tum-Growth online tool (https://kroemerlab.shinyapps.io/TumGrowth/). The log rank test was used for Kaplan-Meier (KM) survival analyses. Correlations were assessed using Spearman's rank correlation analysis. Unless indicated otherwise, statistical analyses were performed by GraphPad Prism 8.

TABLE 1
List of Representative Antibodies used in the present disclosure

| Antibody | Source | Identifier |
|---|---|---|
| Anti-ACBP/DBI antibody | Abcam | ab231910 |
| Anti-POR antibody | Abcam | ab180597 |
| Anti-KEAP1 antibody | Abcam | ab119403 |
| Anti-CHAC1 antibody | Abcam | ab217808 |
| Anti-NCOA4 antibody | Abcam | ab86707 |
| Anti-ACSL4 antibody | Abcam | 234167 |
| Anti-ACSL3 antibody | Abcam | ab151959 |
| Anti-GPC3 antibody | Thermo Fisher | MA517083 |
| | Scientific | |
| Anti-BMAL1 antibody | Abcam | ab93806 |
| Anti-Ki67 antibody | Abcam | ab119403 |
| Anti-PCNA antibody | Abcam | ab217808 |
| Anti-alpha 1 | Abcam | ab86707 |
| Fetoprotein antibody | | |
| Anti- Cytokeratin | Abcam | ab52625 |
| 19 antibody | | |
| Anti-GPX4 antibody | Cell Signaling | CST52455 |
| | Technology | |
| Anti-GAPDH antibody | Cell Signaling | 2118 |
| | Technology | |
| Anti-MAP1LC3B antibody | Cell Signaling | 2775 |
| | Technology | |
| Anti-p62/SQSTM1 antibody | Abnova | H00008878-M01 |
| Goat Anti-Rat | Southern | 3050-05 |
| IgG (H + L) | Biotech | |
| secondary antibody | | |
| Goat Anti-Rabbit | Southern | 4050-05 |
| IgG (H + L) | Biotech | |
| secondary antibody | | |
| IgG2a antibody (in | Bioxcell | BE0085 |
| vivo isotype control) | | |
| Monoclonal anti-ACBP/DBI | Fred Hutch Antibody | N/A |
| (in vivo neutralization) | Technology | |

1. A method of treating cancer in a subject in need thereof, the method comprising administering to the subject:
(a) an agent directed against extracellular human diazepam binding inhibitor (anti-DBI agent) in an amount sufficient to inhibit extracellular human DBI; and
(b) one or more anti-cancer agents,
wherein administration of the anti-DBI agent and the one or more anti-cancer agents is in a therapeutic amount sufficient to treat cancer and results in enhanced treatment of the cancer in the subject as compared to a comparable method in the absence of the administration of the anti-DBI agent.

2. The method of claim 1, further comprising administering one or more immunotherapy agents, wherein administration of the anti-DBI agent and the one or more immunotherapy agents is sufficient to improve a therapeutic effect of immunotherapy relative a comparable method in the absence of the administration of the anti-DBI agent.

3. The method of claim 1, further comprising administering one or more corticotherapy agents.

4. The method of claim 3, wherein the method reduces the immunosuppressive effects of the one or more corticotherapy agents in the subject undergoing immunotherapy or treatment of cancer relative to a comparable method in the absence of the administration of the anti-DBI agent.

5. A method of reducing the immunosuppressive effects of corticotherapy in a subject undergoing immunotherapy, the method comprising administering to the subject:
(a) an agent directed against extracellular human diazepam binding inhibitor (anti-DBI agent) in an amount sufficient to inhibit extracellular human DBI; and
(b) one or more corticotherapy agents;
wherein administration of the anti-DBI agent and the one or more corticotherapy agents is sufficient to reduce the immunosuppressive effects of the corticotherapy in the subject undergoing immunotherapy relative to a comparable method in the absence of the administration of the anti-DBI agent.

6. The method of claim 1, wherein the anti-DBI agent is an antibody or an aptamer.

7. The method of claim 1, wherein the one or more anti-cancer agents comprise a chemotherapy agent, an immunotherapy agent, or both.

8. The method of claim 7, wherein the chemotherapy agent comprises an immunogenic cell death (ICD) inducing activity.

9. The method of claim 7, wherein the immunotherapy agents comprise activity against an immune checkpoint.

10. The method of claim 9, wherein the immune checkpoint comprises PD1 (programmed death 1), PDL1 (programmed cell death-ligand 1), CTLA4 (cytotoxic T-lymphocyte-associated protein 4), PDL2 (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG, CBLB (Casitas b-lineage lymphoma Proto-Oncogene B), CISH (Cytokine Inducible SH2 Containing Protein), or any combinations thereof.

11. The method of claim 7, wherein the immunotherapy agent comprises an anti-PD1 agent, anti-PD-L1 agent, anti-CTLA4 agent, anti-PD-L2 agent, anti-KIR agent, anti-B7-H3 agent, anti-B7-H4 agent, anti-BTLA agent, anti-LAG3 agent, anti-TIM-3 agent, anti-VISTA agent, anti-ILT2/LILRB1 agent, anti-ILT3/LILRB4 agent, anti-ILT4/LILRB2 agent, anti-TIGIT agent, anti-NKG2A agent, anti-PVRIG agent, anti-CBLB agent, anti-CISH agent, or any combinations thereof.

12. The method of claim 3, wherein the one or more corticotherapy agents are selected from cortisol, cortisone, prednisone, prednisolone, methylprednisolone, dexamethasone, betamethasone, triamcinolone, deflazacort, fludrocortisone acetate, deoxycorticosterone acetate, aldosterone, beclomethasone, and any combinations thereof.

13. The method of claim 1, wherein the cancer comprises an inhibitory tumor microenvironment.

14. The method of claim 1, wherein the method further comprises inducing at least one immunosurveillance biomarker comprising: increased CD8⁺ expression, decreased CD4⁺ expression, decreased T_{reg} cells (CD4⁺Foxp3⁺), an increased ratio of CD8⁺ T cells to T_{reg} cells (CD4⁺Foxp3⁺), increased T_{H}+ cells (CD4⁺Foxp3⁻), decreased expression of Lag3⁺ in T_{H}+ cells (CD4⁺Foxp3⁻), decreased expression of Lag3⁺ in CD8⁺ cells, decreased expression of Foxp3⁺ in CD4⁺ cells, decreased T_{reg} ICOS⁺ GITR⁺ Lag3⁻ CD4⁺ cells, or combinations thereof, in a sample relative to a comparable method in the absence of the administration of the anti-DBI agent.

15. The method of claim 1, wherein the method further comprises inhibiting of at least one cancer progression biomarker comprising: a decreased cancer amount, decreased proliferation of cancer cells, increased cancer cell death, decreased cancer growth, or combinations thereof, of the subject relative to a comparable method in the absence of the administration of the anti-DBI agent.

16. The method of claim 1, wherein the cancer is a refractory cancer.

17. The method of claim 1, wherein the cancer is resistant to immune checkpoint inhibitor therapy.

18. The method of claim 1, wherein the cancer is a malignant cancer.

19. The method of claim 1, wherein the cancer is selected from carcinomas, lung cancer, non-small cell lung cancer, and breast cancer.

20. A therapeutic regimen for treating cancer comprising:
(a) an agent directed against extracellular human diazepam binding inhibitor (anti-DBI agent) in an amount sufficient to inhibit extracellular human DBI; and
(b) one or more anti-cancer agents,
wherein the anti-DBI agent and the one or more anti-cancer agents are present in the therapeutic regimen in an amount sufficient to inhibit one or more cancer progression biomarkers in a subject upon administration to the subject relative to a comparable regimen absent the administration of the anti-DBI agent.


SEQUENCE LISTING (3 sequences)

SEQ ID NO: 1 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

SEQ ID NO: 2 - DNA, 634 bp, Homo sapiens
  source 1..634: mol_type = genomic DNA
gctcgcccgagcagggttggggcgagtggaccgcgcctctaaaggcgcttgccagtgcaatctgggcgatcgcttcctggtcctcgcctcctccgctgtctccctggagttcttgcaagtcggccaggatgtctcaggctgagtttgagaaagctgcagaggaggttaggcaccttaagaccaagccatcggatgaggagatgctgttcatctatggccactacaaacaagcaactgtgggcgacataaatacagaacggcccgggatgttggacttcacgggcaaggccaagtgggatgcctggaatgagctgaaagggacttccaaggaagatgccatgaaagcttacatcaacaaagtagaagagctaaagaaaaaatacgggatatgagagactggatttggttactgtgccatgtgtttatcctaaactgagacaatgccttgtttttttctaataccgtggatggtgggaattcgggaaaataaccagttaaaccagctactcaaggctgctcaccatacggctctaacagattaggggctaaaacgattactgactttccttgagtagtttttatctgaaatcaattaaaagtgtatttgttactttaaataactttaaaaaaaaaa

SEQ ID NO: 3 - DNA, 30 bp, synthetic construct
  source 1..30: mol_type = other DNA
tttttttttttttttttttttttttttttt